(19) United States
(12) Patent Application Publication
(10) Pub. No.: US 2009/0198231 A1
(43) Pub. Date: Aug. 6, 2009

(54) METHODS TO TREAT UNWANTED TISSUE WITH ELECTRIC PULSES

(75) Inventors: Axel T. Esser, Muenchen (DE); Thiruvallur R. Gowrishankar, Acton, MA (US); Kyle C. Smith, Cambridge, MA (US); Stephen K. Burns, Durham, NH (US); James C. Weaver, Sudbury, MA (US)

Correspondence Address: K&L Gates LLP, STATE STREET FINANCIAL CENTER, One Lincoln Street, BOSTON, MA, 02111-2950, US

(73) Assignee: MASSACHUSETTS INSTITUTE OF TECHNOLOGY, Cambridge, MA (US)

(21) Appl. No.: 12/329,571

(22) Filed: Dec. 6, 2008

Related U.S. Application Data
(60) Provisional application No. 61/005,675, filed on Dec. 6, 2007.

(51) Int. Cl. A61B 18/14

(52) U.S. Cl. 606/41

(57) ABSTRACT

Provided are methods for selecting parameters of an electrical pulse for electroporation to induce apoptosis in a tissue in need of therapeutic removal in a patient. Also provided are methods and apparatuses for treating a disease by inducing apoptosis in a tissue in need of therapeutic removal in a patient. Further provided are computer-readable media having instructions for selecting parameters of an electrical pulse for electroporation to induce apoptosis in a tissue in need of therapeutic removal in a patient.

### REFERENCE TO RELATED APPLICATIONS

This application claims priority to and the benefit of U.S. Provisional Patent Application No. 61/005,675, filed Dec. 6, 2007, the entire contents of which are hereby incorporated by reference herein.

### STATEMENT REGARDING FEDERALLY SPONSORED RESEARCH

This invention was made with government support under grant number NIH R01-GM63857 from the National Institutes of Health. The United States Government has certain rights in the invention.

### FIELD OF THE INVENTION

The invention relates generally to the field of medical treatments to remove unwanted cells and tissues by applied electrical currents. In particular, the invention relates to the removal of tissue in vertebrates, and the induction of apoptosis by electric pulses.

### BACKGROUND

Interventions that remove unwanted tissue for medical and/or cosmetic reasons in humans and animals are widely recognized to be important. At the cellular level cancer cells frequently do not respond to pharmaceutical treatments because of acquired multiple drug resistance by active pump mechanisms, inhibitors of apoptosis and inhibition of signaling molecules. Molecular mechanisms inhibiting apoptosis pathways can also hinder physical therapies such as localized ionizing radiation as well as systemic pharmaceutical interventions. At the multicellular level there are additional barriers within solid tumors to drug therapies, which arise from the inability of drugs to fully penetrate abnormal, heterogeneous and irregularly vascularized tumor tissue, and thereby reach all of the cancer cells at therapeutic levels. Accordingly, local physical therapies that universally kill all cells within a selected tissue volume are of great interest.

Programmed cell death via apoptosis plays a central role in cell biology and molecular medicine. Mitochondrial outer membrane permeabilization (MOMP), as controlled by Bcl-2 family proteins, is a key biochemical mechanism in apoptosis activation. MOMP involves pores formed by pro-apoptotic Bcl-2 family members, leading to the release of cytochrome-c (cyt-c) and other death molecules from the mitochondria. Death molecules, such as Cytochrome-c, Smac/DIABLO, AIF, EndoG, and Omi/HTRAN2, are molecules which are usually contained in the mitochondlia, but may be released from the mitochondria by a biochemical mechanism that leads to MOMP, such that, if these death molecules are present in the cytosol of the cell in a sufficient number, the cell will undergo cell death by apoptosis. Many tumors and cancer cells have developed a resistance to MOMP and/or the release of death molecules, such that a treatment by, for example, chemotherapy will not lead to cell death. Hence, a plurality of drugs are being developed to overcome the resistance to MOMP and death molecule release. The present invention aims at providing a purely physical method to overcome resistance to MOMP and to death molecule release.

Mitochondria can release more than 50 proteins from the intramembrane space. MOMP and the associated release of death molecules are pivotal events that induce the mitochondrial pathway of apoptosis. In particular, cytolchrome-c elicits the dATP-dependent oligomerization of apaf1, recruits and stimulates caspase-9 and thereby allows for the activation of caspase-3, which leads to apoptosis. Smac/DIABLO and Omi/HtrA both enhance caspase activation through neutralization of inhibitors of apoptosis proteins (IAP). Endonuclease G (endo-G) and flavoprotein apoptosis-inducing factor (AIF) both translocate to the nucleus and promote DNA fragmentation, chromatin condensation, and cell death without caspase activation. Dozens of transducing signaling molecules act on mitochondria to induce or inhibit MOMP.

Cancer cells develop resistance to chemical and physical therapies by acquiring molecular mechanisms for inhibiting MOMP and therefore apoptosis. Cancer cells disable the mitochondrial pathway of apoptosis by overexpressing anti-apoptotic Bcl-2 proteins. Thus cancer chemotherapy and radiation therapy, indirect methods that cause apoptosis upstream of DNA damage via transcription factor p53, face a dilemma because they fail if cells are apoptosis-resistant.

The therapeutic induction of MOMP can restore apoptosis in cancer cells, and currently developed cancer therapeutics indeed aim to eliminate tumors by inducing apoptosis. Electrical release of mitochondlial death molecules is a direct physical intervention and has potential for screening cells for MOMP by comparing responses to candidate drug responses, and may also have application in localized drug-free solid tumor treatment where drug penetration is often incomplete.

What is needed is a direct physical method to induce apoptosis by electrical mitochondrial outer membrane (MOM) permeabilization. The present invention addresses this need.

### SUMMARY OF INVENTION

Various aspects and embodiments of the invention are described in detail herein. Each embodiment can apply to any aspect.

In one aspect, the invention provides a method for selecting parameters of an electrical pulse for electroporation to induce apoptosis in a tissue in need of therapeutic removal in a patient. The method includes the steps of generating an equivalent electrical model of the tissue; calculating from the model the number of death molecules, which are released from all MOM pores in each cell in the tissue for an electrical pulse; and selecting an electric pulse that will produce a number of released death molecules sufficient to initiate apoptosis in the tissue. In various embodiments, the calculations are performed using a computer. In some embodiments, the death molecules can be at least one of Cytochrome-c, Smac/DIABLO, AIF, EndoG, and Omi/HTRAN2. In various embodiments, the electric pulse has a time constant, field strength, rise time, fall time, and/or pulse form. In some embodiments, the number of death molecules released is sufficient to achieve caspase-3 activation in the tissue. In some embodiments, the calculation step is performed using a transport lattice equivalent electrical model of the tissue. In some embodiments, the step of calculating the number of released death molecules is performed using death molecule current through a MOM pore, and the death molecule current for cytochrome-c can be given by

$${I}_{pore}^{cyt - c} = {\frac{π {r}_{p}^{2} {F}^{2} {Δψ}_{m}^{MOM} {D}_{cyt - c} {z}_{cyt - c}^{2}}{{RTd}_{m}} [ cyt - c ]}_{i} ,$$

In some embodiments, the number of released death molecules sufficient to initiate apoptosis is based on a concentration of death molecules in the cell of the tissue.

In various embodiments, the methods of the invention include the step of calculating the time constant, field strength, rise time, fall time, and/or pulse form of the electric pulse which is capable of causing the release of a minimum number of death molecules (e.g., cytochrome-c) sufficient to induce apoptosis in one or more cells of the tissue. In various embodiments, the pulse form of the electric pulse is selected from the group consisting of exponential, trapezoidal, triangular, ramped, and bipolar shaped.

In another aspect, the invention provides a method for treating a disease by inducing apoptosis in a tissue in need of therapeutic removal in a patient. The method includes the steps of generating an equivalent electrical model of the tissue; calculating from the model the number of death molecules, which are released from all MOM pores in each cell in the tissue for an electrical pulse; selecting an electric pulse having a time constant, field strength, and pulse form that will produce a number of released death molecules sufficient to initiate apoptosis in said tissue; and applying the electric pulse to the tissue.

In another aspect, the invention provides a method for selecting parameters of an electrical pulse for electroporation to induce apoptosis in a tissue in need of therapeutic removal in a patient. The method includes the steps of generating a model of the tissue; calculating the minimum number of cytochrome-c molecules (or other death molecules) that must be released from the mitochondrial outer membrane (MOM) in order to achieve caspase-3 activation in the tissue; calculating the number of cytochrome-c molecules released from a single MOM pore of sufficient size to release cytochrome-c; calculating the number of MOM pores of the size sufficient to release cytochrome-c that are needed to release the minimum number of cytochrome-c molecules from the MOM in order to achieve caspase-3 activation in the tissue; and calculating the time constant, field strength, rise time, fall time, and pulse form of the electric pulse that will produce the number of MOM pores of the size sufficient to release cytochrome-c that are needed to release the minimum number of cytochrome-c molecules using the model of the tissue. In some embodiments, the calculations are performed using a computer. In some embodiments, the step of calculating the number of MOM pores of the size sufficient to release cytochrome-c is performed using the transport lattice of the cylindrical cell system model. In some embodiments, the step of calculating the number of cytochrome-c molecules released from a single MOM pore of sufficient size to release cytochrome-c is performed using the equation:

$${I}_{pore}^{cyt - c} = {\frac{π {r}_{p}^{2} {F}^{2} {Δψ}_{m}^{MOM} {D}_{cyt - c} {z}_{cyt - c}^{2}}{{RTd}_{m}} [ cyt - c ]}_{i} ,$$

In various embodiments, the step of calculating the minimum number of cytochrome-c that must be released from the MOM in order to achieve caspase-3 activation is calculated using a cytochrome-c to apaf-1 association constant of 4×10⁷ M⁻¹, and a minimum number of activated apaf-1 of 1 nM.

In another aspect, the invention provides a method for treating a disease by inducing apoptosis in a tissue in need of therapeutic removal in a patient. The method includes the steps of generating a model of the tissue; calculating the minimum number of cytochrome-c molecules (or other death molecules) that must be released from the mitochondrial outer membrane (MOM) in order to achieve caspase-3 activation in the tissue; calculating the number of cytochrome-c molecules released from a single MOM pore of sufficient size to release cytochrome-c; calculating the number of MOM pores of the size sufficient to release cytochrome-c that are needed to release the minimum number of cytochrome-c molecules from the mitochondrial outer membrane (MOM) in order to achieve caspase-3 activation in the tissue; calculating the time constant, field strength, rise time, fall time, and pulse form of the electric pulse that will produce the number of MOM pores of the size sufficient to release cytochrome-c that are needed to release the minimum number of cytochrome-c molecules using the model of the tissue; and applying an electric pulse to the tissue in response to the time constant, field strength and pulse form calculations. In some embodiments, the pulse form of the electric pulse is selected from the group consisting of exponential, trapezoidal, triangular, ramped, and bipolar. In some embodiments, the duration of the electric pulse is time constant τₚᵤₗₛₑ greater than 1.0 microsecond and less than 100 microsecond. In various embodiments, the magnitude of the applied electric field E⁰ₐₚₚ is greater than 5 kV/cm and not more than about 40 kV/cm. In various embodiments, the death molecule released is, for example, cytochrome-c, Smac/DIABLO, AIF, EndoG, Omi/HTRA2, and combinations thereof. In various embodiments, more than one electric pulse per treatment is used. In various embodiments, more than one electric pulse (e.g., 1, 2, 3, 4, 5, 6, 7, 8, 9, 10, or more) is administered per treatment, such as, for example between 1 and 100 electric pulses; every integer between 1 and 100 is specifically contemplated herein. In some embodiments, electric pulses are administered at an intra-treatment interval of at least about one hour to at least about two days (e.g., at least one application of current per hour to at least one application of current per two days); each treatment interval between about one hour and about two days is specifically contemplated herein. In various embodiments, the electric pulses are administered in a number and at a frequency sufficient to allow time to allow phagocytes to engulf apoptotic cells.

In another aspect, the invention provides a computer-usable medium having computer readable instructions stored thereon to perform a method for selecting parameters of an electrical pulse for electroporation sufficient to induce apoptosis in a tissue in need of therapeutic removal in a patient. Computer-usable (i.e., computer-readable) media are well known in the art and include, by way of non-limiting examples, hard disks (i.e., hard drives) and removable media (e.g., CDs, DVDs, flash drives, diskettes, and the like). A person of skill in the art will appreciate that any number of suitable computer-usable media can be used. The method includes the steps of generating a model of the tissue in need of therapeutic removal; calculating the minimum number of cytochrome-c molecules (or other death molecules) that must be released from the mitochondrial outer membrane (MOM) in order to achieve caspase-3 activation in the tissue; calculating the number of cytochrome-c molecules released from a single MOM pore of sufficient size to release cytochrome-c; calculating the number of MOM pores of the size sufficient to release cytochrome-c that are needed to release the minimum number of cytochrome-c molecules from the mitochondrial outer membrane (MOM) in order to achieve caspase-3 activation in the tissue; and calculating the time constant, field strength and pulse form of the electric pulse that will produce the number of MOM pores of the size sufficient to release cytochrome-c that are needed to release the minimum number of cytochrome-c molecules using the model of the tissue.

In another aspect, the invention provides an apparatus for treating a patient by causing apoptosis in a tissue in need of therapeutic removal. The apparatus includes a means for generating a model of the tissue; a means for calculating the minimum number of cytochrome-c molecules (or other death molecules) that must be released from the mitochondrial outer membrane (MOM) in order to achieve caspase-3 activation in the tissue; a means for calculating the number of cytochrome-c molecules released from a single MOM pore of sufficient size to release cytochrome-c; a means for calculating the number of MOM pores of the size sufficient to release cytochrome-c that are needed to release the minimum number of cytochrome-c molecules from the mitochondrial outer membrane (MOM) in order to achieve caspase-3 activation in the tissue; a means for calculating the time constant, field strength and pulse form of the electric pulse that will produce the number of MOM pores of the size sufficient to release cytochrome-c that are needed to release the minimum number of cytochrome-c molecules using the model of the tissue; and a means for applying an electric pulse to the tissue.

In another aspect, the invention provides a method for treating a tissue of a patient. The method includes the step of selecting an electric pulse waveform for electroporation of the tissue with a field strength of 2-40 kV/cm and a duration of 1 μs-100 μs to induce apoptosis in the tissue (e.g., 10 kV/cm, 30 μs; 15 kV/cm, 30 μs; 20 kV/cm, 10 μs), and the step of applying the selected electric field pulse waveform to the tissue. These field strengths will lead to cell size independent electroporation, and these field strength—field duration combinations are a compromise of having a desired effect inside the cells, e.g. release of death molecules, without causing a significant temperature increase of the tissue. In some embodiments, the method further includes the step of determining an intracellular electric condition in the tissue for at least one electric pulse waveform. In some embodiments, the selecting of the electric pulse waveform is based on the intracellular condition. In various embodiments, the intracellular condition includes at least one of: an intracellular electric field in at least one cell of the tissue; an intra-organelle electric field in at least one organelle of a cell in the tissue; a number of pores and/or a size of pores in at least one membrane of an organelle in a cell in the tissue; a number of pores and/or a size of pores in at least one mitochondria in a cell in the tissue; a number of pores and/or a size of pores in at least one outer mitochondrial membrane in a cell in the tissue; or a number of at least one sort of death molecules released from at least one mitochondria in the tissue.

In some embodiments, the at least one sort of death molecule includes, for example, at least one of Cytochrome-c, Smac/DIABLO, AIF, EndoG, and Omi/HTRAN2. In some embodiments, the electric pulse waveform includes at least one electric pulse. In some embodiments, the electric pulse waveform includes a plurality of electric pulses. In some embodiments, each of the plurality of electric pulses is separated in time. In some embodiments, the plurality of electric pulses are applied at an intra-treatment interval of at least one hour to at least about two days (e.g., at least one application of current per hour to at least one application of current per two days); each treatment interval between about one hour and about two days is specifically contemplated herein. In various embodiments, the method includes the further step of applying a surfactant to the tissue. In some embodiments, the plurality of electric field pulses are applied to the tissue in such a way, that it allows phagocytes to engulf apoptotic cells in the tissue.

In another aspect, the invention provides a method for selecting an electric pulse waveform for electroporation of a tissue. The method includes the steps of generating an equivalent electric model of the tissue; and determining an intracellular condition in the equivalent electric model during a plurality of electric pulse waveforms. In various embodiments, the intracellular condition includes at least one of: an intracellular electric field in at least one cell of the tissue; an intra-organelle electric field in at least one organelle of a cell in the tissue; a number of pores and/or a size of pores in at least one membrane of an organelle in a cell in the tissue; a number of pores and/or a size of pores in at least one mitochondria in a cell in the tissue; a number of pores and/or a size of pores in at least one outer mitochondrial membrane in a cell in the tissue; or a number of at least one sort of death molecules released from at least one mitochondria in the tissue.

In another aspect, the invention provides a computer-usable medium having computer readable instructions stored thereon to perform a method for selecting parameters of an electrical pulse for electroporation sufficient to induce apoptosis in a tissue in need of therapeutic removal in a patient. The method includes the steps of generating an equivalent electrical model of the tissue in need of therapeutic removal; calculating from the model the number of death molecules, which are released from all MOM pores in each cell in the tissue for an electrical pulse; and selecting an electric pulse that will produce a number of released death molecules sufficient to initiate apoptosis in the tissue.

In another aspect, the invention provides an apparatus for treating a patient by causing apoptosis in a tissue in need of therapeutic removal. The apparatus includes a means for generating an equivalent electrical model of the tissue; a means for calculating from the model the number of death molecules, which are released from all MOM pores in each cell in the tissue for an electrical pulse; and a means for selecting an electric pulse that will produce a number of released death molecules sufficient to initiate apoptosis in the tissue.

In some embodiments, the at least one sort of death molecule can be, for example, at least one of Cytochrome-c, Smac/DIABLO, AIF, EndoG, and Omi/HTRAN2. In some embodiments, the method includes the further step of generating a plurality of the electric pulse waveforms. The electric pulse waveform includes at least one electric pulse, and the parameters of the at least one electric pulse include at least one of a rising time, falling time, electric field strength, pulse shape, pulse time constant, and/or duration. The at least one electric pulse shape can include at least one of an exponential, trapezoidal, triangular, ramp, or bipolar pulse shape. In some embodiments, the method includes the further step of selecting an electric pulse waveform from the plurality of electric pulse waveforms in response to at least one of the intracellular conditions.

In another aspect, the invention provides an apparatus for treating a tissue of a patient, the apparatus being connectable to at least one electrode. The apparatus includes a means for selecting an electric pulse waveform for electroporation of a tissue with a field strength of 2-40 kV/cm and a duration of 1 μs-100 μs to induce apoptosis in the tissue as well as a means for applying the selected electric field pulse waveform to the tissue. In some embodiments, the apparatus further includes a means for storing a plurality of electric pulse waveforms. In various embodiments, the apparatus further includes a means for monitoring the application of the selected electric pulse waveform on the tissue (e.g., measuring electrical impedance for the applied electric field waveform and/or measuring temperature in or close to the treated tissue region). In various embodiments, the apparatus further includes a means for generating a plurality of electric pulse waveforms. In various embodiments, the selection means is adapted to select the electric pulse waveform based on a predetermined intracellular condition in the tissue. In various embodiments, the selection means is adapted to select the electric pulse waveform to either cause apoptosis or necrosis in the tissue. In some embodiments, the electric pulse waveform includes at least one electric pulse, and in some embodiments the electric pulse waveform includes a plurality of electric pulses, and the plurality of electric pulses can be separated in time. In various embodiments, the plurality of electric pulses are applied at an intra-treatment interval of at least one hour to at least about two days (e.g., at least one application of current per hour to at least one application of current per two days).

In another aspect, the invention provides a computer program that implements a method according to any of the aspects or embodiments described herein.

In another aspect, the invention provides a computer usable medium having computer readable instructions stored thereon to perform a method according to any of the aspects or embodiments described herein.

In another aspect, the invention provides a variable high voltage high current pulse generator. The variable high voltage high current pulse generator includes a first discharging terminal and a second discharging terminal, a plurality of switched current supplying modules, a high voltage charging supply having high voltage supply positive and negative charging terminals, and a control circuit having a control bus. Each of the switched current supplying modules has a first positive charging terminal and a second negative charging terminal, a first discharging terminal and a second discharging terminal, and a control bus. The positive charging terminal of each of the plurality of switched current supplying modules is connected to the high voltage supply positive charging terminal of the high voltage power supply. The negative charging terminal of each of the plurality of switched current supplying modules is connected to the high voltage supply negative charging terminal of the high voltage power supply. The control bus of each of the plurality of switched current supplying modules is connected to the control bus of the control circuit. In some embodiments, the control bus of each of the plurality of switched current supplying modules can include several control means. The second discharging terminal of each of the plurality of switched current supplying modules, except a last of the plurality of switched current supplying modules, is connected to the first discharging terminal of the subsequent one of the plurality of switched current supplying modules. The second discharging terminal of the last of the plurality of switched current supplying modules is the second discharging terminal of the high voltage high current pulser. The first discharging terminal of the first of the plurality of switched current supplying modules is the first discharging terminal of the high voltage high current pulser.

In some embodiments, the variable high voltage high current pulse generator includes a current source, a half bridge, a first switch, and a second switch. In various embodiments, the current source can include, for example, a capacitor. The half bridge has a first terminal, a second terminal, and a third terminal. The first switch, in a first state, connects one side of the current source to the charging terminal of the switched current supplying module; and, in a second state, connects the same side of the current source to the first terminal of the half bridge. The second switch, in the second state, connects the other side of the current source to the second terminal of the half bridge. The third terminal of the half bridge is the second discharge terminal of the switched current supplying module.

In some embodiments of the variable high voltage high current pulse generator, each of the plurality of switched current supplying modules further includes a rise-time control module having an input terminal and an output terminal, and a fall-time control module having an input terminal and an output terminal. The half bridge can further include a fourth terminal and a fifth terminal, where the fourth terminal of the half bridge is connected to the output terminal of the rise-time control module and the fifth terminal of the half bridge is connected to the output terminal of the fall-time control module.

In some embodiments of the variable high voltage high current pulse generator, the input terminal of the rise-time control module is in electrical communication with the control terminal of the switched current supplying modules, and the input terminal of the fall-time control module is in electrical communication with the control bus of the switched current supplying modules.

In another aspect, the invention provides a method of supplying a controlled pulse to an electrode. The method includes the steps of charging a current source, discharging the current source through an electrode at a predetermined rise-time for a first period of time, and discharging the current source through the electrode at a predetermined fall time for a second period of time. In some embodiments, the step of discharging the current source through the electrode at the predetermined rise-time for the first period of time includes controlling a voltage at the gate of a transistor. In some embodiments, the step of discharging the current source through the electrode at the predetermined fall-time for the second period of time includes controlling a voltage at the gate of a transistor.

In another aspect, the invention provides a method of supplying a pulse of electricity with predetermined characteristics to an output electrode (i.e., an electrode which contacts a target patient tissue). The method includes the steps of, for each of a plurality of switched current supplying modules, charging a current source within each of the switched current supplying modules, discharging the current source of each of the switched current supplying modules through the electrode at a predetermined rise-time for a first period of time, and discharging the current source of each of the switched current supplying modules through the electrode at a predetermined fall-time for a second period of time.

### BRIEF DESCRIPTION OF DRAWINGS

The invention is pointed out with particularity in the appended claims. The advantages of the invention described herein, together with further advantages, may be better understood by referring to the following description taken in conjunction with the accompanying figures.

FIG. 1A shows a system model of a cell, FIG. 1B shows local circuit models for electrolyte and membrane, and FIG. 1C shows a circuit representation of a dynamic electroporation model, in accordance with an illustrative embodiment of the present invention.

FIGS. 2A-C are a series of graphs showing 1D membrane patch response to exponential pulse, in accordance with an illustrative embodiment of the present invention.

FIG. 3 is a graph showing pore energy as a function of pore radius, in accordance with an illustrative embodiment of the present invention.

FIGS. 4A-D are a series of graphs showing electrical response of a cell model to exponential pulse, in accordance with an illustrative embodiment of the present invention.

FIGS. 5A-E are a series of graphs showing intracellular electric response to exponential pulse, in accordance with an illustrative embodiment of the present invention.

FIGS. 6A-D are a series of graphs showing electric response of a cell model to exponential pulse, in accordance with an illustrative embodiment of the present invention.

FIGS. 7A-E is a series of graphs showing electropermeability and number of transported molecules as a function of time for trapezoidal-shaped conventional electroporation, in accordance with an illustrative embodiment of the present invention.

FIGS. 8A-E is a series of graphs showing permeability and number of transported molecules as a function of time for trapezoidal supra-electroporation pulse, in accordance with an illustrative embodiment of the present invention.

FIG. 9 is a diagram showing a model of apoptosis by mitochondrial outer membrane electroporation (MOMEP), in accordance with an illustrative embodiment of the present invention.

FIGS. 10A-B are a series graphs showing electric response, pore sizes, and emergence of organelle electroporation, in accordance with an illustrative embodiment of the present invention.

FIG. 11A is a series of graphs showing mitochondrial outer member (MOM) pore distribution and radius, and FIG. 11B is a series of graphs showing the number of cyt-c release pores, in accordance with illustrative embodiments of the present invention.

FIG. 12 is a series of graphs showing the response of a cell model to an 8.1 kV/cm, 40 μs exponential pulse, in accordance with an illustrative embodiment of the present invention.

FIG. 13 is a series of graphs showing the response of a cell model to a 2 kV/cm, 40 μs exponential pulse, in accordance with an illustrative embodiment of the present invention.

FIG. 14 is a series of graphs showing the response of a cell model to a 4 kV/cm, 40 μs exponential pulse, in accordance with an illustrative embodiment of the present invention.

FIG. 15 is a series of graphs showing the response of a cell model to a 6 kV/cm, 40 μs exponential pulse, in accordance with an illustrative embodiment of the present invention.

FIG. 16 is a series of graphs showing the response of a cell model to a 10 kV/cm, 40 μs exponential pulse, in accordance with an illustrative embodiment of the present invention.

FIG. 17 is a series of graphs showing the response of a cell model to a 16 kV/cm, 40 μs exponential pulse, in accordance with an illustrative embodiment of the present invention.

FIG. 18 is a series of graphs showing the response of a cell model to a 12 kV/cm, 18 μs bipolar pulse, in accordance with an illustrative embodiment of the present invention.

FIG. 19 is a series of graphs showing the response of a cell model to a 12 kV/cm, 10 μs bipolar pulse, in accordance with an illustrative embodiment of the present invention.

FIG. 20 is a series of graphs showing the response of a cell model to a 10 kV/cm, 10 μs trapezoidal pulse, in accordance with an illustrative embodiment of the present invention.

FIG. 21 is a series of graphs showing the response of a cell model to a 12 kV/cm, 3 μs trapezoidal pulse, in accordance with an illustrative embodiment of the present invention.

FIG. 22 is a series of graphs showing the response of a cell model to a 16 kV/cm, 20 μs trapezoidal pulse, in accordance with an illustrative embodiment of the present invention.

FIG. 23 is a series of graphs showing the response of a cell model to a 25 kV/cm, 300 ns trapezoidal pulse, in accordance with an illustrative embodiment of the present invention.

FIG. 24 is a series of graphs showing the response of a cell model to a 20 kV/cm, 2 μs pulse, in accordance with an illustrative embodiment of the present invention.

FIG. 25 is a series of graphs showing the response of a cell model to a 30 kV/cm, 2 μs pulse, in accordance with an illustrative embodiment of the present invention.

FIG. 26 is a series of graphs showing the response of a cell model to a 20 kV/cm, 3 μs pulse, in accordance with an illustrative embodiment of the present invention.

FIG. 27 is a series of graphs showing the response of a cell model to a 14 kV/cm, 10 μs pulse, in accordance with an illustrative embodiment of the present invention.

FIG. 28 is a series of graphs showing the response of a cell model to a 16 kV/cm, 10 μs pulse, in accordance with an illustrative embodiment of the present invention.

FIG. 29 is a series of graphs showing the response of a cell model to an 8 kV/cm, 30 μs pulse, in accordance with an illustrative embodiment of the present invention.

FIG. 30A shows a cell model, and FIGS. 30B-C are a series of graphs showing the response of a cell model to 20 V/cm small magnitude trapezoidal pulses, in accordance with an illustrative embodiment of the present invention.

FIG. 31 shows a block diagram of a pulser, in accordance with an illustrative embodiment of the present invention.

FIG. 32 shows a diagram of a pulser module, in accordance with an illustrative embodiment of the present invention.

### DETAILED DESCRIPTION

The present relates to methods to remove unwanted cells based on apoptosis, which is a programmed cell death. While apoptosis in cells is naturally done by biochemical stimuli, the method of the invention induces apoptosis by a physical method, and in particular by using appropriate electric pulse waveforms. The present inventions relate to the creation of electrical electric field waveforms (electric pulses) that cause the release of death molecules from the mitochondria in sufficient numbers to lead to apoptosis of the cells.

For this purpose, an equivalent electrical model of cells and tissues is described to predict the physical response of tissue cells (i.e., redistributed fields via equipotentials, membrane potentials, dynamic pore population evolution) to different electric pulse waveforms, and the resulting biochemical changes (i.e., the amounts of cytochrome-c released and the activation of caspase-3 that usually forces apoptosis). The response of a single cell within a tissue, with the plasma membrane (PM), double membrane nuclear envelope (NE), endoplasmic reticulum membrane (ERM), and three mitochondria with an inner mitochondrial membrane (MM or MIM) and an outer mitochondrial membrane (OMM and MOM), can be described by this model.

The present invention in one embodiment relates to an in silico (computer based) spatially distributed single cell model that contains the most important organelles, and shows with this model that conventional electroporation (EP) not only alters the PM, but can also lead to significant intracellular fields. These intracellular fields can cause voltage gating of organelle channels, and for larger fields, even electroporation of organelle membranes. This cell system model discloses that electric pulses may affect cells towards either necrotic or apoptotic cell death. This single cell model may be used in combination with tissue models to determine the intracellular electric conditions in a tissue of many cells.

In other embodiments two- and three-cell system models which have for each cell a PM and two mitochondria with IMM and OMM, as in the above single cell model are described. In these multicellular cell system models, all membranes have spatially distributed resting potential sources that create appropriate resting potentials. Specifically ΔΨ_{MOM}=0, ΔΨ_{PM}=−90 mV, and ΔΨ_{MM}=−200 mV. Simulated EP in these cell system models creates local dynamic pore distributions (pore distributions, n(r)), that depend on time and the transmembrane voltage at each local area of a membrane. This causes a spatially varying membrane shunt conductance that decreases ΔΨ, which is often close to zero for a time governed by the pore lifetime (here taken to be 3 ms), taken from measurements on lipid bilayer membranes. These in silico cell system models demonstrate that certain electric field waveforms are particularly useful in removing unwanted cells in vivo, and can therefore be used to remove unwanted tissues in patients in need of such tissue removal.

The invention relates to a method to select the parameters of an electric pulse that is sufficient to produce electroporation to induce apoptosis in a multicellular tissue. This method to select the parameters of an electric pulse includes the steps of generating a model of the tissue; calculating the minimum number of cytochrome-c molecules (or other death molecules) that must be released from the mitochondrial outer membrane (MOM) in order to achieve caspase-3 activation in the tissue; calculating the number of cytochrome-c molecules released from a single MOM pore of sufficient size to release cytochrome-c, and calculating the number of MOM pores of the size sufficient to release cytochrome-c that are needed to release the minimum number of cytochrome-c molecules from the MOM in order to achieve caspase-3 activation in the tissue; and calculating the time constant, field strength and pulse form of the electric pulse that will produce the number of MOM pores of the size sufficient to release cytochrome-c that are needed to release the minimum number of cytochrome-c molecules using the model of the tissue. In preferred embodiments, the step of calculating the number of MOM pores of the size sufficient to release cytochrome-c is performed using the transport lattice of the cylindrical cell system model. In some embodiments, the step of calculating the number of cytochrome-c molecules released from a single MOM pore of sufficient size to release cytochrome-c is performed using the equation

$${\frac{{I}_{pore}^{cyt - c} = π {r}_{p}^{2} {F}^{2} {Δψ}_{m}^{MOM} {D}_{cyt - c} {z}_{cyt - c}^{2}}{{RTd}_{m}} [ cyt - c ]}_{i} ,$$

In some embodiments, the step of calculating the minimum number of cytochrome-c molecules that must be released from the MOM in order to achieve caspase-3 activation is calculated using a cytochrome-c to apaf-1 association constant of 4×10⁷ M⁻¹, and a minimum number of activated apaf-1 of 1 nM. The invention further relates to a method for treating a patient in need of tissue removal, which includes the steps of selecting parameters of an electric pulse for electroporation of the tissue in need of removal to induce apoptosis in the tissue; and applying the electric pulse with the selected parameters to the tissue in need of removal. In preferred embodiments, the parameters of the electric pulses are selected using the methods of the invention to generate an electric pulse that is sufficient to produce electroporation and thereby induce apoptosis in a multicellular tissue. Examples of parameters for an electric pulse suitable for the treatment methods of the invention include, but are not limited to, 8.1 kV/cm, 40 μs exponential pulse; 10 kV/cm, 40 μs exponential pulse; 16 kV/cm, 40 μs exponential pulse; 12 kV/cm, 18 μs bipolar pulse; 10 kV/cm, 10 μs trapezoidal pulse; 12 kV/cm, 3 μs trapezoidal pulse; 16 kV/cm, 20 μs trapezoidal pulse; 20 kV/cm, 2 μs, (0.2 μs rise time+1.6 μs+0.2 μs fall time); 30 kV/cm, 2 μs, (0.2 μs+1.6 μs+0.2 μs); 20 kV/cm, 3 μs, (0.3 μs+2.4 μs+0.3 μs); 14 kV/cm, 10 μs, (1 μs+8 μs+1 μs); 16 kV/cm, 10 μs, (1 μs+8 μs+1 μs); and 8 kV/cm, 30 μs, (3 μs+24 μs+3 μs).

The invention further relates to a method for treating a multicellular tissue in need of removal, the tissue having a plurality of cells of a plurality of sizes, which includes administering one or more electric pulses of an applied voltage and duration sufficient to cause electroporation of at least one organelle sufficient to release death molecules from the organelle and cause apoptosis. In one embodiment, the electric pulse is described by the equation:

Eₐₚₚ(t)=E⁰ₐₚₚexp[−t/τₚᵤₗₛₑ].

In preferred embodiments, the time constant τₚᵤₗₛₑ of the electric pulse is greater than 1.0 microsecond and less than 100 microseconds, and/or the applied voltage E⁰ₐₚₚ of the electric pulse is greater than 5 kV/cm but not more than about 40 kV/cm. Each value between 1.0 and 100 microseconds is specifically contemplated herein.

In other embodiments, the organelle from which the death molecules are released is a mitochondria. Death molecules of particular interest include, but are not limited to, cytochrome-c, Smac/DIABLO, AIF, EndoG, and Omi/HTRA2. A death molecule of particular interest is cytochrome-c.

In some embodiments of the method for treating a multicellular tissue, more than one electric pulse per treatment is administered. The time intervening between the administration of electric pulses is, in some embodiments, at least about one hour, and in preferred embodiments, at least about several hours. In some embodiments, the time between the administration of the electric pulses may be about a day or more for the convenience of the patient. The time between the pulses should be sufficient to allow for the clearance of apoptotic cells by phagocytosis, such as by macrophages and dendritic cells in order to minimize or eliminate tissue inflammation and immunosuppressive effects following the onset of apoptosis by mitochondrial outer membrane electroporation (MOMEP). In one embodiment, more than one session of pulse application is employed with sufficient time between the administration of the electric pulses to provide time for phagocytes, such as macrophages, to migrate into the region where the electrical treatment induces cells to begin apoptosis, as described by Savill et al. and Ravichandran and Lorenz in 2007. In some embodiments, not only should further electrical pulsing await clearance of apoptotic cells, the time between electric pulses should be sufficient to allow the phagocytes to migrate out of the treatment area, as this allows the phagocytes to be spared. While not limiting the method of the invention to a particular mechanism, it is contemplated that allowing sufficient time between electric pulses to clear apoptotic cells will generally avoid inflammation and scarring, and will have particular usefulness in situations where removal of unwanted tissue involves tissue sites that are near delicate structures or are readily visible on the patient.

In some embodiments, the parameters of the electric pulses may differ between pulses of a treatment of multiple pulses. In some embodiments, the treatment method is administered by first using a waveform that causes MOMEP in a subpopulation of the tissue to be removed. In some embodiments, the waveform in a first session has a combination of smaller magnitude and/or longer duration within the range of pulse waveform parameters sufficient to induce the release of death molecule(s) from the mitochondria in the tissue to be removed. Then, in these embodiments, in a subsequent session at least one hour later, or the next day or later, a subsequent electric pulse waveform is administered that is somewhat larger and/or longer than used in the first session. While not limiting the mechanism of action of these embodiments, this subsequent administration may result in MOMEP sufficient to release enough death molecules in a cell subpopulation that has survived the treatment of the first session. In some embodiments, a sequence of further electric pulse sessions is used, as needed. The total number of electric pulses administered to a multicellular tissue may be, in various embodiments, from 1 to about 100. Every integer between 1 and 100 is specifically contemplated herein.

In other embodiments, the spatial distribution of tissue level electric fields is varied between treatment sessions by controlling the voltage or current pulse waveform magnitude that is applied to two or more electrodes, so as to spatially target cell subpopulations that survive the preceding treatment session. This embodiment provides different electric field spatial gradients by applying different electrical stimuli to various electrodes, so that this embodiment applies different magnitudes and durations of intracellular electric field waveforms within the exposure region for treating tissue to be removed. While not limiting the mechanism of action of the invention, it is contemplated that these different electric fields interact with the cells in the exposure region, such that some cells (a subpopulation) have mitochondria which experience MOMEP so as to evolve pore populations that have enough pores of sufficient size (radius) for long enough times to release enough death molecules to cause apoptosis.

It is known that a cell with PM pores due to EP may be resealed by the intravenous injection of a nonionic surfactant, polaxamer 188 (P188) before electrical pulsing to prevent leakage of biochemicals from the cell such that the cell does not die by necrosis. In one embodiment of the treatment method of the invention, one or more nonionic surfactants are administered to the multicellular tissue in need of removal before and/or after the administration of an electric pulse. In some embodiments, the nonionic surfactant is administered by intravenous injection of surfactant, or by injection of surfactant into or near the tissue to be removed. In preferred embodiments, the electric pulse is administered before any significant loss of surfactant by diffusion within the interstitial space and by perfusion clearance via blood capillaries. While not limiting the mechanism of action of the invention, it is contemplated that in these embodiments, the time can be extended in which apoptotic cells retain biochemicals and minimize leakage, before phagocytes such as macrophages and dendritic cells arrive, and that inflammation and undesired immune effects can be minimized.

The electrical stimulus source, connections from the source to electrodes, and electrodes for applying electric field waveforms are general, and well known. Electrical stimuli are widely used to create electric fields for various purposes within cell suspensions in vitro and within living tissue in vivo, which are hereby incorporated by reference. Briefly, electrical and electronic devices are well known that apply voltages to two or more electrodes, pass current through two or more electrodes, or deliver charge through two or more electrodes. In this case of EP the most common source is a voltage pulse source and connecting wires (or other conductors). Needle or wire electrodes suitable for in vivo tissue EP have been widely discussed, and involve well known materials. The electric field is applied to a specific tissue region by at least one electrode (e.g., 1, 2, 3, 4, 5, 6 or more electrodes). In particular, a target tissue region with a particular shape may be treated by arranging a suitable number of electrodes inside the target tissue region, the suitable number being dependant on the particular shape and size of the tissue region as well as the number and arrangement of electrodes used. The optimization of electrode placement and numbers is well known in the alt.

In general, it is expected that numerous target tissue types can be treated in vivo in accordance with the present invention, thereby destroying the target tissue, because cells in a multicellular tissue will be electroporated in a way that does not depend on the size of the cells. This is because the pulse is adapted to electroporate all cells of the tissue during the initial charging phase of the plasma membrane and the initial charging phase is independent of the size of the cell. Thus, it is the combination of particular pulse field strengths and pulse durations that enable a cell-size independent means for inducing apoptosis using electrical pulses. In particular, skin malignancies, such as skin cancer (e.g., melanomas) are expected to be treatable in accordance with the present invention due to the accessibility of the relevant target tissue. Further examples of human and animal tissues that may be treated by this method include, without limitation, basal cell carcinoma, squamous cell carcinoma, and neuroendocrine carcinoma in skin in particular, as well as breast, bladder, colon and rectal, endometrial, kidney, lung, non-Hodgkin lymphoma, pancreatic, prostate, and thyroid cancers in general.

A transport lattice (TL) method can be used to generate an equivalent electrical model of a tissue, which allows for an equivalent description of electrical, chemical, and thermal behavior in a complex biological geometry that may contain cells of different sizes, cells of different types, inhomogeneities, and anisotropies.

Examples of an equivalent electrical model of a tissue are provided herein for a multicellular model of many irregular cells in a tissue and a tissue model for a larger scale, wherein their electrical and thermal responses to any electroporation pulse may be determined. Each system model represents rabbit liver tissue, but on a different spatial scale. This method of generating an equivalent electric model of a tissue can be applied to any type of model, and a person skilled in the art with a knowledge of electric parameters of the tissue to be treated will readily understand to generate an equivalent electric model of any desired tissue.

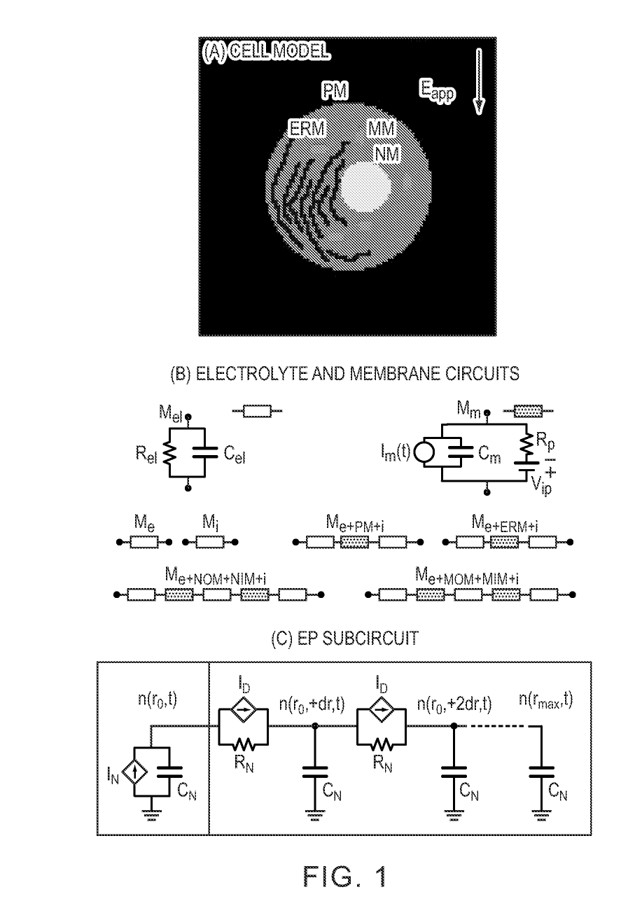

Multicellular Model of many irregular cells in a tissue. Multicellular geometry may be based on a drawing motivated by a tissue section image. For example, a layer of 20 liver cells with 14% interstitial fluid volume may be considered. Hepatocytes have an average cell size diameter of 21.7 μm. A corresponding TL with 101 nodes times 101 nodes may be constructed as a large electric circuit comprising equivalent electric models for passive charge transport and storage (resistors, capacitors) within electrolytes and active elements (pumps, electroporation) at the membranes of the cells and the cell organelles. Linked local membrane and electrolyte models, as shown in FIG. 1B for the electrolyte (“e”) and the intracellular electrolyte (“i”) are connected to their nearest neighbors on a Cartesian lattice. The local membrane models may be interconnected at regularly spaced nodes, with submodels that represent the plasma membrane PM, the endoplasmic reticulum ERM, the nucleus NOM, NIM and the mitochondria MOM, MIM, each with two contacting regions of electrolyte (FIG. 1B). There is no transport in the z-direction in this two-dimensional model, but the equivalent model may be expanded into the z-direction to allow molecular and ionic transport in this direction. The lattice spacing, as well as the depth of the system model may be 1 μm. Voltages applied along the top and bottom boundary of the system model provide the applied uniform field from the applied electric pulse and the electric waveform.

Multiscale Tissue Model for large scale A larger scale tissue model can comprise a large tissue region and two ideal cylindrical electrodes, each with a radius of, for example, re=0.25 mm, separated by, for example, Le=10 mm. The nominal applied electric field, Eapp may be defined here as the voltage difference between the electrodes, Vapp, divided by the electrode spacing, Le. Although needle electrodes actually have spatially varying fields, the term nominal electric field may be used for convenience, and it is also in line with the original definition in the limit of infinite needle radii (i.e., planar electrodes). This tissue model with, for example, a scale of 200 mm times 200 mm is symmetric about y=0. Nodes can be optimally distributed throughout the tissue model using a meshing algorithm. The multiscale tissue model may account for the electrical response at both the microscopic (e.g., PM electroporation) and macroscopic (e.g., needle geometry) scales and the interplay between the two. The scale of the tissue system is orders of magnitude larger than the scale of an individual cell and therefore a discretization of the system could not realistically resolve individual cells and membranes. The multiscale model, however, may use representative simple cell models distributed throughout the system model to calculate the local cell and membrane response, as well as the intracellular electric field and the macroscopic electrical transport properties are determined by the distributed models. In the equivalent circuit for each cell in the tissue model, there is one electrical circuit node on each side of the intracellular space, and the electrical potentials of these nodes are calculated at all time points. At any given time point, the intracellular electric field magnitude can be calculated as the difference between these potentials (in magnitude) divided by the distance separating them (i.e., the size of the cell). From the determined intracellular electric field, any intracellular condition, such as organelle electroporation, pore sizes at the organelle membranes, molecular transport through pores in the organelles, and in particular death molecule release from the mitochondria, may be calculated.

The impedance of a region of tissue is equal to the impedance of a cell scaled to have the same relative dimensions, assuming that tissue comprises a uniform grid of such cells. Thus, a simple cell model can be used, which has a membrane enclosed region of intracellular electrolyte surrounded by extracellular electrolyte. This simple model can be translated into an equivalent electrical circuit. The membrane and each region of electrolyte have an associated conductivity and permittivity. Additionally, each electrolyte region can have a tortuosity to account for the structural complexity of tissue to be treated, and which is not otherwise represented by the model. The relative sizes can be chosen, for example, such that 14% of the total volume is extracellular, but any other value adapted to the tissue to be treated may be assumed. The tortuosities may be used as free parameters in fitting the frequency-dependent tissue conductivity to measured values, for example, the rat liver tissue conductivity to that measured experimentally. By this choice of electrical and geometrical parameters, the model may reproduce the static conductivity value and approximately the same trend in the frequency dependence. The equivalent circuit for the simple cell model is placed between each pair of adjacent nodes in the mesh with electrical components scaled to the local mesh geometry. The effective conductivity of the membrane may thus change in accordance with the local degree of membrane electroporation as determined by the distributed cell models.

Further, an equivalent circuit for a single cell can be created for each node in the tissue model to determine the cellular response to the local electric field. Each of these circuits can be distinct from the primary, macroscopic tissue-level circuit network, but all of the circuits are solved simultaneously. The asymptotic model of electroporation with no pore expansion or the Smoluchowsli model of electroporation with pore expansion can be used to determine the electric response at the membrane of the cells and the membrane of the organelles in the tissue. The voltage across the cell unit is equal to the local electric field magnitude, as determined from the electric potential of the nodes in the mesh, multiplied by the cell unit length. Thus, the distributed cell models determine the transmembrane voltage, the intracellular electric field, the pore density and the pore sizes throughout the tissue domain as functions of time. The pore density and the pore sizes may then determine the membrane conductivity used in the macroscopic transport network. Thus, there is provided a continual feedback between the macroscopic (tissue level) and microscopic (cellular level) models. That is, the macroscopic behavior determines the local electric field in the microscopic model and the subsequent behavior at the microscopic scale (e.g., for electroporation) then determines the local electrical properties at the macroscopic scale.

Electrolyte and Membrane Models. The passive electric components for the electrolyte can be resistors and capacitors, as shown in FIG. 1B. Those circuit elements can include components for charge storage and conduction, resting potential, and the asymptotic or SE model of electroporation. This method thus provides convenient means for combining the dm=5 nm thick membrane with a TL of the much larger tissue scale. The dielectric constant of the extra- and intracellular electrolytes can be, for example, 72. Close to a pore the membrane dielectric may be treated as pure lipid and assigned a dielectric constant of, for example, 2.1. This choice can recognize that local membrane properties are relevant to pore formation. In contrast, the membrane capacitance of the plasma membrane and the organelle membrane involve a spatial average over membrane lipid and protein regions resulting in a relative permittivity of, for example, 5 for the PM. The extracellular electrolyte may have a conductivity of, for example, 1.2 S/m while the medium inside the cell has a conductivity of, for example, 0.4 S/m. Further, a simplified, single resting potential source model comprised of an active voltage source, Vip, and source series resistance, Rip, can be used. The fixed quantities Vip and Rip can determine the membrane resting potential in the absence of any applied fields.

TL Solution. The system model circuits may be solved for the electric potential by means of Kirchhoffs laws using any circuit solver such as, for example, Berkeley SPICE 3f5 (University of California, Berkeley, Calif.). SPICE generates solutions that can be processed and displayed as, for example, equipotentials and distributions of electroporated regions.

Specific Absorption Rate. The specific absorption rate (SAR), defined as Joule heating divided by the tissue (electrolyte) mass density, can be used in the electrical characterization of any tissue to time-varying electromagnetic fields. That is, for the selection of an electroporation waveform, additional information that relates to a temperature rise can be generated and used for the selection of a pulse waveform that leads to apoptosis in the tissue while temperature increase remains limited.

### EXAMPLES

### Example 1

### Cell and Organelle Electropermeabilization and Molecular Transport Through Dynamic Nanopores

To study the action of electromagnetic fields in biological systems, cell and tissue membranes are often characterized by passive conductive and dielectric properties. Conventional EP pulses involve characteristic times (pulse duration, rise and fall times) that exceed the PM charging time τ_{PM}, which is typically 0.1 to 1 μs, for mammalian cells in suspension. If the passive models were sufficient, then the cell interior would be essentially shielded from the external electric field as the PM is completely polarized for exposure times exceeding τ_{PM}. However, this conclusion neglects nanoscale membrane reorganizations associated with the process of EP, a robust and universal mechanism by which cells, depending on their size, respond to electric fields of magnitudes larger than about 10⁴ V/m. EP is widely used in biological research and is also a tool for several medical interventions. Examples include loading of molecules into cells, in particular gene therapy by DNA transfection, electrochemotherapy and delivery of plasmid DNA to treat solid tumors, transport of drugs through tissue barriers such as skin, high-throughput siRNA delivery and screening of gene-specific silencing by RNA interference, but also the application of EP pulses without any drugs present that lead to a controlled biological effect.

Although conventional EP has been used widely for a quarter century a quantitative understanding of EP has not yet been achieved and controversies remain about the underlying molecular mechanisms. This is in part due to the sparsity of quantitative experimental results and also in part to biological diversity that, sometimes, leads to contradictory conclusions when different cell systems and their environment are compared. Various embodiments show that conventional EP is not a local effect at the PM only, as is often stated, but also leads to yet not appreciated intracellular interactions that may stimulate and modify organelles. Intracellular bioelectric interactions are important for regeneration, morphogenesis, and left-right patterning because the cell interior itself contains a complex electric signaling circuit. Embryonic development is controlled by electrophoretic molphogen gradients inside cells and perturbations cause randomization of gene expression. Cell organelles are capable of generating and conveying electric signals. Mitochondria are prominent examples, and their disfunction underlies many diseases. A persistent challenge is how external electric fields can initiate or override intracellular signals and therefore cause a robust biological effect. Endogeneous wound electric fields as small as 10 V/m are, for example, able to induce a biochemical cascade inside cells that involves gene interaction, but the initiating mechanism remains unknown.

Rapid intracellular manipulation by applications of nanosecond pulses with magnitudes on the order of 10⁷ V/m and characteristic times significantly shorter than τ_{PM} are designed to achieve high intracellular electric fields during exposure times less than τ_{PM}, conditions presumably not seen by cells in nature. Supra-EP then occurs in all the cell's membranes, but pores remain small and therefore unable to transport significant amounts of conventional EP markers or nucleic acids. Molecular dynamics (MD) simulations of small, finite-size membrane patches under these extremely high field strengths provide detailed molecular information that elucidates the complex dipolar interplay of lipids, water, and ions during EP, and confirm the expectation of pore formation, validate earlier ideas about pore geometries, and demonstrate, for example, the translocation of charged lipids across a pore. Yet the challenge remains to transfer MD results into an improved mechanistic understanding that can be incorporated into cell level models.

A multiscale approach to generate an equivalent electric model can be based on transport lattices (TLs) which represent a plurality of cell models with relevant organelles and includes a dynamic EP model. Dynamic pore size behavior in tissue under conditions of irreversible EP can be studied with this approach. The results can provide (i) quantitative predictions comparing different EP pulse protocols, and (ii) new insight into electric conditions inside a cell and at the PM, from which a mechanistic basis for direct intracellular electric effects may be inferred. In particular, conventional EP can lead to significant internal electric fields, which can gate ubiquitous voltage-dependent organelle channels and can even cause organelle EP, providing a counter example to the assertion that only pulses with time scales shorter than τ_{PM} can achieve intracellular effects. The quantitative results for the electropermeability of the PM and molecular transport through dynamic nanopores, as response to conventional and supra-EP pulses are shown. In spite of a virtually symmetric electropermeability of the PM, transport of charged molecules through nanopores is predominantly asymmetric. Thus molecular transport on this spatial scale continues to reveal novel properties, and is important for synthetic nanopore technology that pursues DNA sequencing or nanofluidics.

Methods

Modular, multiscale transport lattices in biology. The three-dimensional TL maps the biological system model (FIG. 1A) onto an equivalent electrical circuit and is based on local models for charge transport, storage, sinks, and sources, and thus allows for the assessment of the spatial distributions of fields, potentials, and membrane pores on the cellular level (microdosimetry). Nanometer-scale membrane models Mₘ (FIG. 1B) are constructed from modules that represent resistive phospholipid regions, resting potential sources, and local electrical capacitances. The EP mechanism is included in Mₘ via EP-subcircuits interacting locally at all respective membrane sites. These modules are assigned to local membrane areas and connected to their nearest micrometer-scale electrolyte neighbors by Mₑₗ models (FIG. 1B) to form a Cartesian TL. Features of the TL method are described in detail above. The TLs here have ˜2×10⁴ interconnected local models and are solved by Kirchhoff's laws, using Berkeley SPICE version 3f5 (University of California, Berkeley, Calif.).

FIG. 1A shows a cross-sectional geometry and system model of cell (radius 10 μm) with organelles (ERM, mitochondria, and nucleus); indicating direction of external electric field. FIG. 1B shows a local circuit models for electrolyte (Mₑₗ; open rectangle) and membrane (Mₘ; filled rectangle) assembled in different configurations to represent the electrolytes and electrolyte/membrane interfaces (bottom two rows of the panel). FIG. 1C shows an equivalent circuit representation of the dynamic EP model, distinguishing the asymptotic EP model (pore creation and destruction; left box) and its extension to the full Smoluchowski equation (pore expansion and contraction; right box). The current source I_{N} is the pore creation and destruction term in Equation (2). The effective voltage on each capacitor C_{N} represents the pore distribution n(rₚ, t) for different pore radii (not a physical voltage in the system model). Pore drift and diffusion are related to the current source I_{N} and the resistor R_{N}, respectively. The local pore distribution in subcircuit shown in FIG. 1C determines the pore conductivity and hence the membrane current Iₘ as input in Mₘ (FIG. 1B).

System model for the cell with organelles. The cylindrical cell system geometry (2D cross section shown in FIG. 1A; system depth d_{ys}=0.35 μm) includes the PM (10 μm radius), nuclear membrane (NM; 3 μm radius), the endoplasmic reticulum membrane (ERM) and, in close proximity to the ERM, five mitochondria (approximated with 1 μm×2 μm cross sectional area), each with MOM and MIM, separated by 15 nm of intermembrane space (FIG. 1A). Invaginated christae are represented by making the effective MIM area a factor f_{A}=5 larger than the MOM. The nucleus has two membranes separated by 10 nm, and encloses the cisterna. A passive resistance is assigned to the membranes, lower values for both NM's and MOM account for their leaky nature. The ERM is continuous with the nucleus and has an irregular shape. The dielectric constant of the extra- and intracellular electrolytes is 80. Close to a pore the membrane is treated as pure lipid and assigned a dielectric constant of 2.1. In contrast, PM and organelle membrane capacitance values involve a spatial average over lipid and protein regions, leading to a dielectric constant of 5. A simplified resting potential source model comprising an active voltage source, Vᵢₚ, and a series source resistance, Rᵢₚ, is used to represent approximately the effect of ion pumps and channels in creating membrane resting potentials Δψ_{PM,rest}: −90 mV, 90 mV, and −200 mV for PM, ERM, and MIM, respectively; Δψ_{PM,rest} for MOM is zero. To account for both the continuity of the nucleus with the endoplasmic reticulum and the overall zero resting potential of the nucleus opposite resting potentials of ±Δψ_{ERM,rest} were assigned at the outer and inner NM. Voltages applied at virtual electrodes at the top and bottom of the system boundary provide the uniform applied field.

Dynamic EP model. Cell membranes change their permeability dramatically at elevated transmembrane potentials by EP. This biophysical mechanism is hypothesized to involve transient aqueous pores and their creation, destruction, expansion, and contraction may be described by the Smoluchowski equation (SE). The Smoluchowski equation for the time-dependent pore distribution n(rₚ, t) is

$$\frac{\partial n}{\partial t} = \frac{\partial }{\partial {r}_{p}} [ {D}_{p} ( \frac{\partial n}{\partial {r}_{p}} + \frac{n}{{k}_{B} T} \frac{\partial W}{\partial {r}_{p}} ) ] . \qquad ( 1 )$$

where rₚ is pore radius, W is pore energy, D is diffusion constant and T is temperature. From n(rₚ, t) drₚ, the number of pores between radius rₚ and rₚ+drₚ, and by integration the total number of pores in any local membrane area can be found. Further, the pore-radius dynamics depends on the diffusion constant in pore radius space Dₚ, and the pore energy W(rₚ, Δψ(t))=Wₘ+Wₑₗ contains mechanical (Wₘ) and electrical (Wₑₗ) parts, which are known in the art. The mechanical part describes the contribution of mechanical energies, such as surface tension and line tension, to the pore energy, while the electrical part Wₑₗ takes into account electrical contributions, such as dielectric effects, into account and depends on the transmembrane potential Δψ(t). This procedure allows for the simultaneous treatment of small and large pores. Pore formation is a kinetic process and can plausibly be described by a rate equation, were the number of minimum-size pores N_{p,min}=n(r_{p,min}) drₚ with r_{p,min}=0.8 nm is obtained from

$$\frac{\partial {N}_{p , m i n} ( {r}_{p , m i n} , t )}{\partial t} = A {e}^{B {Δψ}^{2} / {k}_{B} T} - \frac{{N}_{p , m i n} ( {r}_{p , m i n} , t )}{{τ}_{p}} . \qquad ( 2 )$$

Equation (2) includes experimentally determined parameters A and B. The time scale of pore formation varies and depends on pulse conditions, waveforms and on membrane geometry. Hence there is no absolute threshold for EP as often suggested in the literature. Reported pore lifetimes vary from milliseconds to minutes. At present, there is no understanding of the basic mechanisms that give rise to this wide range of lifetimes. Large pores need to relax to minimum-sized conducting pores before decaying, but this process occurs on a us time scale according to the present model of EP, which is much faster than is observable. There is thus also no known reason that shorter pulses should give rise to shorter pore lifetimes, as previously suggested. An illustrative experimental value is τₚ=3 ms. Initial conditions for Equations (1) and (2) are based on the equilibrium pore number per local area N_{p,min}^{eq}=Aτₚ, that further defines a local equilibrium pore distribution n^{eq}(rₚ, t). Asymptotic models of EP neglect pore expansion and shrinkage (Dₚ=0). This is sufficient for nanosecond electric field pulses but not for longer conventional EP pulses.

The asymptotic EP model, as given by Equation (2), has been represented by an equivalent circuit. An extended circuit to account for full SE pore dynamics is shown in FIG. 1C. The first term of Equation (1) describes diffusion in pore radius space and is represented by a resistor R=(drₚ)²/Dₚ between succeeding pore radii rₚ and rₚ+drₚ, with drₚ=0.05 nm the spacing of the pore space discretization. The drift-term in Equation (1) is represented by an active current element

$${I}_{{r}_{p} , {r}_{p} + {dr}_{p}} = \frac{{D}_{p}}{2 {k}_{B} {T ( {dr}_{p} )}^{2}} [ n ( {r}_{p} + {dr}_{p} ) + n ( {r}_{p} ) ] [ W ( {r}_{p} + {dr}_{p} ) - W ( {r}_{p} ) ] . \qquad ( 3 )$$

The expression ∂n(rₚ,t)/∝t|_{drift}=I_{rp-drp,rp}−I_{rp,rp+drp} contains both the linear and the quadratic energy derivatives of the SE. The unit capacitor at each pore node is C_{N}=1, and the current source I_{N} represents the rate of change of N_{P,MIN}, as given by the r.h.s. of Equation (2). The dynamic EP circuit is solved at every local membrane site (area Aₘ=0.12 μm²), from which the pore conductivity are obtained and hence the total pore current Iₘ at the PM and all organelle membranes as input into the membrane module Mₘ. Thus the EP circuit interacts locally with the distributed physical transport lattice.

Pulse Waveforms. Exponential pulses Eₐₚₚ(t) E⁰ₐₚₚexp[−t/τₚᵤₗₛₑ] with time constant τₚᵤₗₛₑ are widely used conventional EP waveforms. The applied field for parallel plane electrodes with spacing L is E⁰ₐₚₚ=V⁰ₐₚₚ/L, with the applied voltage Y⁰ₐₚₚ. For an illustrative embodiment, pulses with τₚᵤₗₛₑ=40 μs, which were reported to induce apoptosis in Jurkat T-lymphoblasts and HL-60 cells, and assume a pulse rise time of 1 μs were used. The robustness of the EP mechanism suggests that these conclusions are also valid for other waveforms. The transport results are generated for trapezoidal pulses, comparing a conventional pulse with E⁰ₐₚₚ=1 kV/cm, 100 μs duration, and 1 μs rise and fall times as typically applied in electrochemotherapy protocols with a supra-electroporating pulse of E⁰ₐₚₚ=95 kV/cm, 60 ns duration, and with 1 ns rise and fall times.

### Results

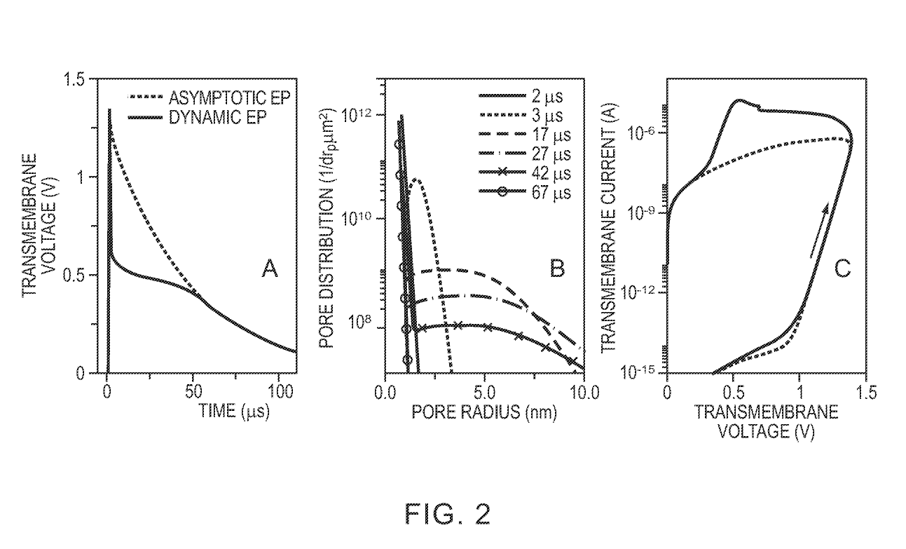

Qualitative nature of dynamic EP in a planar membrane patch. Referring to FIG. 2, which shows a 1D membrane patch (area 3 μm×3 μm) response to exponential pulse(E⁰ₐₚₚ=1 kV/cm, τₚᵤₗₛₑ=40 μs, 1 μs rise time), FIG. 2A Δψ initially charges with time constant τ=0.1 μs and reaches the REB peak at Δψ≈1.4V. The conductance increase due to pore formation and pore expansion then causes a rapid decay to Δψ≈0.5 V. Then, Δψ exhibits a voltage-regulating effect with a plateau-like behavior due to dynamic pore-size changes. The asymptotic EP model (blue) has distinctively different behavior, viz. Δψ follows the time-dependence of Eₐₚₚ(t). FIG. 2B shows time-dependent pore distributions. Created pores at minimum size (r_{p,min}=0.8 nm, dashed line) expand to larger radii and subsequently return to r_{p,min}. The pore distribution evolves during the entire pulse. Referring to FIG. 2C, the membrane current—voltage (I−Δψ) characteristics shows conductance hysteresis (arrow indicates time direction). The strong increase in I above 0.8-1 V marks the EP onset and pore creation. After REB peak, I continues to increase despite a drop in ψ. The lack of pore expansion in the asymptotic model limits I, hence the SE model of EP (black) has larger conductance after the REB peak for the time during voltage regulation.

The PM trans membrane voltage Δψ_{PM} (t) plays a central role in interaction mechanisms that stimulate and modify cells by physiologic electric fields and also for the mechanism of EP at larger fields. FIG. 2 demonstrates the salient features of EP due to an exponential 1 kV/cm, τpulse=40 jus pulse, based on a planar membrane patch model, which is an equivalent electrical model of a lipid bilayer, wherein the lipid bilayer is represented by an equivalent capacity and an equivalent time-dependent (i.e., electroporation-dependent) membrane resistance, and solved by the TL method. FIG. 2A shows that with the onset of the pulse, ΔψPM increases with time constant τ_{PM}=0.1 is. Even though EP sets in at Δψ_{PM} 0.8-1 V, as marked by a strong increase in membrane current (FIG. 2C), the transmembrane potential continues to rise and a burst of pore creation continues until a peak at Δψ_{PM} 1.4 V. The membrane can not maintain this value. Instead, a sudden drop in Δψ_{PM} that is associated with a reversible high conductance state of the membrane (reversible electrical breakdown (REB)) occurs. Expansion of pore radii subsequently causes a further increase in membrane conductivity. Remarkably, Δψ_{PM} does not track the external pulse time-dependence (FIG. 2A). Instead Δψ_{PM} reaches a plateau-like state around Δψ_{PM} 0.5 V.

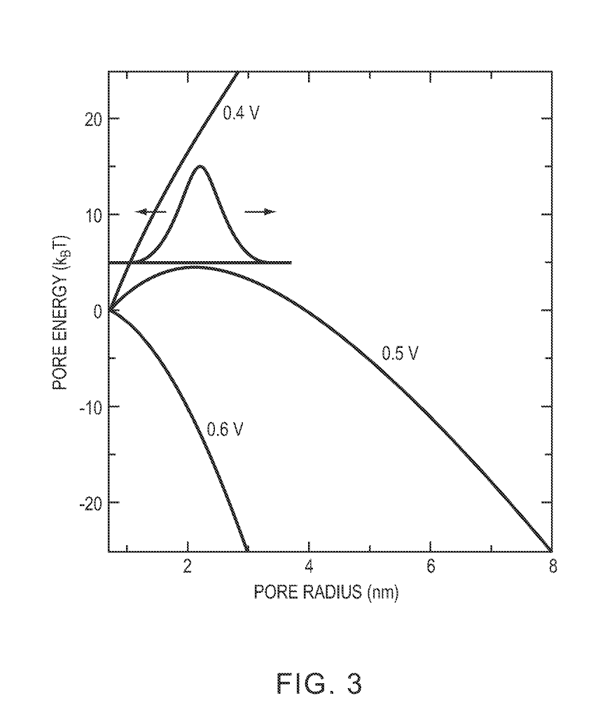

FIG. 3 shows pore energy W (relative to the minimum size pore energy at r_{p,min}=0.8 mm) as function of pore radius for three transmembrane potentials. For Δψ=0.5 V, the plateau value typical for conventional EP, an energy maximum at pore radius of 2.1 nm causes part of the pore distribution (sketched by blue profile) to shift to smaller pore sizes, and simultaneously some part to shift to larger pore sizes. In contrast, lower and higher values of Δψ lead exclusively to pore shrinkage and pore expansion, respectively. Inset shows pore geometry.

At this transmembrane voltage, as demonstrated in FIG. 3, the pore energy has a maximum at a pore radius of about 2 nm such that dynamic change in the pore distribution results simultaneously in both pore shrinkage and pore expansion, and the number of pores with a radius below 2 nm and a above 2 nm increases. A plateau-like state occurs because the applied field decreases during the exponential pulse, which tends to decrease Δψ_{PM} as well. However, previously expanded pores respond by shrinking, which increases the membrane resistance such that voltage division with the electrolyte resistance inhibits a decrease of Δψ_{PM}. This voltage regulation effect fundamentally depends on a Δψ_{PM}(t)—sensitive pore distribution. The finite slope of the Δψ_{PM}—plateau reflects the kinetics of pore growth governed by Dₚ which affects the time needed to change pore sizes. The slope would decrease for higher values of Dₚ, and increase for smaller values of Dₚ. Consequently, the voltage regulation effect is not present if pore size change is inhibited (Dₚ=0) as in asymptotic EP model, for which Δψ_{PM}(t) follows Eₐₚₚ(t). At the end of the plateau, Δψ_{PM}(t) decays exponentially and follows the time-dependence of the external pulse. That shape of the transmembrane voltage: rise, REB peak, sudden drop, plateau, decay, has been observed in many different systems, and are characteristic signatures of EP.

Dynamic changes in the Δψ_{PM}-dependent pore distribution n(rₚ,t) are shown in FIG. 2B. Pore creation occurs at the minimum size r_{p,min}=0.8 nm that allows for electrical conduction. Subsequent expansion and shrinkage of pores are evident as the pulse progresses. The importance of creating larger pores has long been recognized, e.g., pores larger than 5 nm are required for DNA to enter a cell. However, DNA electrophoresis and pore plugging may impose additional forces, and thus augment pore expansion not reflected in the model. After the end of the plateau and during the exponential decay of Δψ_{PM}(t), the pore distribution rapidly (˜μs) shrinks and becomes dominated by minimum-size pores. Hence long-lived pores are not expected to be larger than 1 nm, underlying the observed behavior that larger molecules can only be transported during the pulse, not after.

FIG. 2C illustrates the non-linear membrane current-voltage (I_{PM}−Δψ_{PM}) characteristics, demonstrating hysteretic electric behavior of the membrane, e.g., the possibility of having two different I_{PM} values at the same Δψ_{PM}. EP thus introduces a memory effect, and significant membrane conductance increases occur at both (i) the onset of EP at Δψ_{PM}≈0.8-1 V and (ii) pore expansion after REB (peak at Δψ_{PM}=1.4V). Remarkably, the membrane current I_{PM} continues to grow after the REB peak despite a drop in Δψ_{PM}, which is only possible if pores expand, and cause an increase in pore conductivity. An important interpretation of the model's behavior is that the membrane conductivity changes during the entire pulse as pores dynamically change their size.

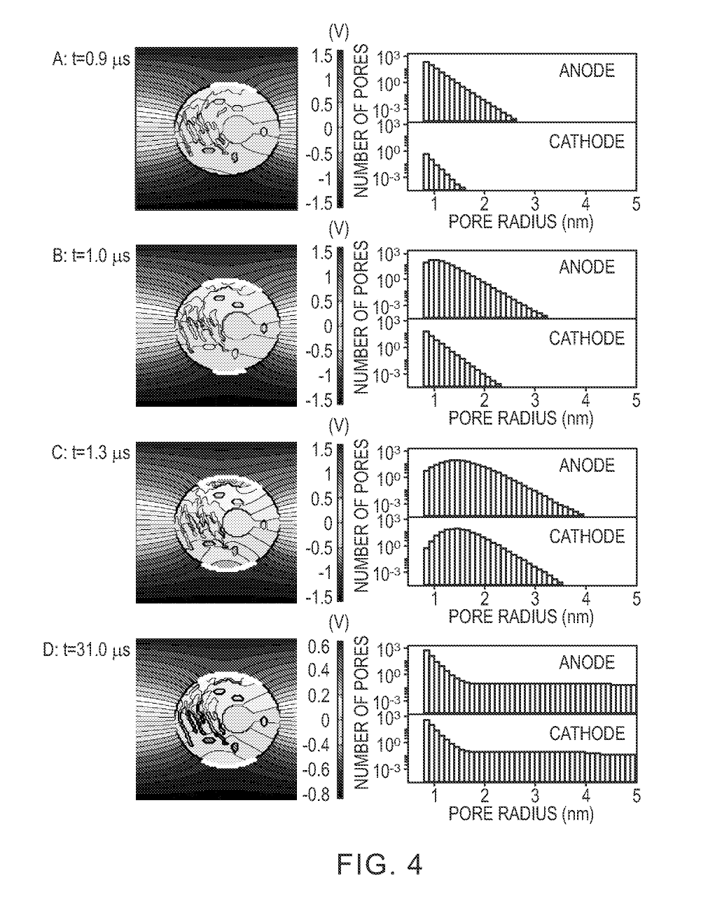

Distributed cellular response to conventional EP. FIG. 4 shows the electrical response of cell model to exponential pulse(E⁰ₐₚₚ=1 kV/cm, τₚᵤₗₛₑ=40 μs, 1 μs rise time); colorbar shows the potential scale. Pore histograms for the anodic and cathodic membrane side give the total number of pores and their size within intervals of 0.1 nm. In FIG. 4A, EP starts at t=0.9 μs on the anodic side (white dots are local membrane sites with ≧1 pore), followed at t=1.0 μs on the cathodic side in FIG. 4B. Pore expansion also starts initially at the anodic side. FIG. 4C shows significant pore expansion at t=1.3 μs at both the anodic and cathodic side; intracellular equipotentials reveal electric fields in the cell interior. In FIG. 4D, at t=31 μs, the pore histograms exhibit maxima at r_{p,min}, but with non-equilibrium tails toward larger pores. Pore histograms are similar, anodic and cathodic side mean pore sizes and pore number differ by about 10%.

The electric response of the cell system model to the exponential pulse (E⁰ₚₚ=1 kV/cm, τₚᵤₗₛₑ=40 μs, rise time 1 μs) is given in FIG. 4. Equipotential lines show the dynamic redistribution of the electric field due to EP at cellular membranes, partially passing through the cell interior. White dots correspond to at least one local pore. In addition, pore histograms of the anodic and cathodic PM sides give the total number of pores within a radius interval of 0.1 nm. Membrane EP is strikingly dynamic. An initial asymmetry in the lateral EP pattern, and highlighted in the pore histograms, is caused by the PM's resting potential source. Specifically the anodic cell side is hyperpolarized and pores are created first at that side at t=0.9 μs (during the pulse rise time). EP of the cathodic cell side follows at t=1 μs and the lateral extent of the electroporated region increases with time. Pore expansion at the anodic side has then already started. Thus EP sets in at different times at the anodic and cathodic poles during the pulse, but the absolute difference in the decreases with increasing field strength because of faster rates of Δψ_{PM} increase. At t=1.3 μs, significant pore expansion has occurred at both PM poles, and thus larger pores become available for molecular uptake and release. Pore expansion after REB leads to a further reduction in Δψ_{PM} and thus the creation of new pores is strongly reduced. Local Δψ_{PM} values at the anodic and cathodic poles are similar (not shown). The electric pore energy Wₑₗ (FIG. 3) decreases and pore shrinkage occurs for most pores even during the pulse. However, a subpopulation of larger pores grows in size during the Δψ_{PM}-plateau. Thus the pore histograms re-achieve a maximum at the minimum pores size (r_{p,min}=0.8 nm), but also maintain long tails extending to large pore radii. Note that a somewhat larger electroporated region appears on the anodic side, and the electroporated region is not symmetric in polar angle (the angle between the electric field direction and membrane site) because of the presence of organelles that are unevenly distributed in the cell model's interior; these perturb and redistribute the intracellular field.

As a result of the cell's resting potential, the total number of PM pores on the anodic side is about 18% less compared with the cathodic side. But even with this pore number difference, the mean pore radius <rₚ> remains equal to within 10% on both sides (anode <rₚ>=1.84 nm, cathode <rₚ>=1.78 nm). Remarkably, this difference (and also the total number of pores) depends strongly on the ionic strength of the electrolytes, and may be even reversed if the extracellular conductivity σₑₓ of the medium exceeds the intracellular conductivity σᵢₙ. The conductivity of physiologic saline (σ=1.2 S/m) is used throughout the whole system. For example, a choice of σₑₓ=5 S/m and σᵢₙ=0.4 S/m leads to 16% more pores on the anodic side. Reported volume averaged intracellular conductivities are three to five times smaller than the extracellular value for physiologic saline, and are due to the crowded environment that excludes electrolyte volume. The presence of organelles is tantamount to having a smaller effective intracellular conductivity, as can be shown from the behavior of pore number reversal, but the inclusion of many more organelles to represent a truly crowded cell interior is not yet computationally feasible.

A consistent EP feature is the experimentally observed asymmetric transport of molecules and dyes entering through either the anodic or cathodic cell hemispheres for monopolar pulses. This asymmetric transport was also associated with asymmetries in the transmembrane voltage Δψ_{PM}, the membrane conductance G_{PM}, and ultimately with the pore distributions nₚ itself, all leading to asymmetric current-voltage behavior. Intriguingly, synthetic nanopores also exhibit asymmetric current-voltage behavior. But this asymmetry is build-in by geometric design of the synthetic pores that are asymmetric in shape. That is not expected for membrane pores, and has not been observed in MD simulations. Four major contributions to asymmetric transport have been proposed: (i) ionic strength of the solution and ionic differences between the cell interior and exterior, (ii) the cell's resting potential, (iii) phospholipid asymmetry, and (iv) an intrinsic membrane property such as the membrane dipole potential. Properties (i-iii) may contribute to asymmetry, but the differences in Δψ_{PM}, G_{PM}, and nₚ between the anodic and cathodic side are rather small and they appear insufficient to explain the observed effects, as asymmetric transport has been shown to occur even for small ions such as Ca²⁺ under experimental conditions in which the inner and outer membrane leaflets have identical composition and a resting potential is absent.

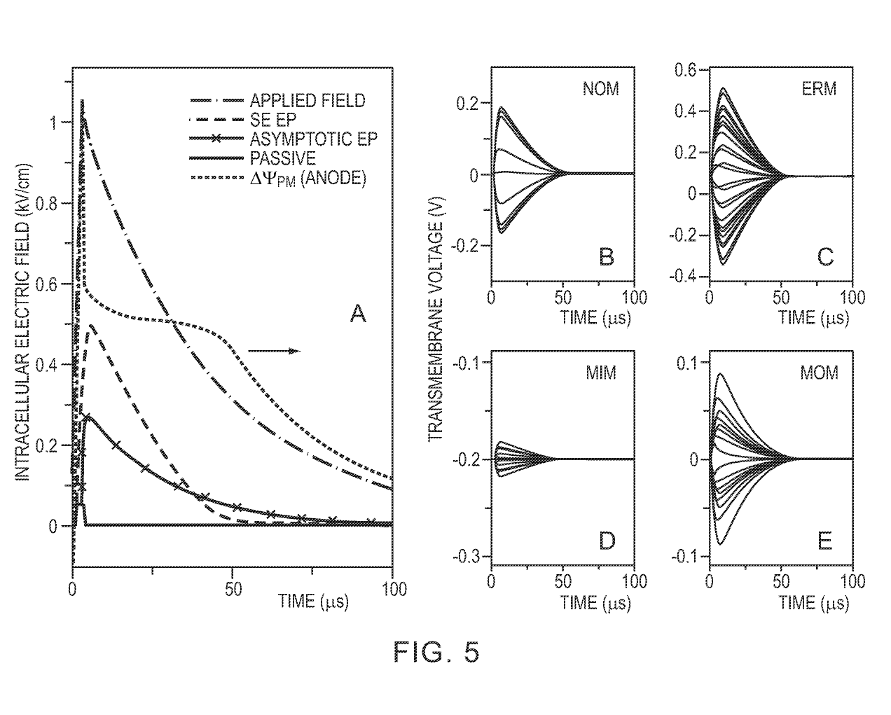

Intracellular electric fields arising from conventional EP. Heterogeneous intracellular electric fields Eᵢₙₜ appear because of PM-EP that leads to a partial redistribution of the applied electric field through the cell (indicated in FIG. 4 by intracellular equipotentials). FIG. 5 shows intracellular electric response to exponential pulse of FIG. 4. FIG. 5A shows the intracellular field Eᵢₙₜ, at the cell center without any organelle present. A passive PM yields essentially zero Eᵢₙₜ except during the brief pulse rise time (black). The asymptotic EP model without pore expansion leads to Eᵢₙₜ values of about 25% of Eₐₚₚ (blue). The SE model has about 50% of the external field due to pore expansion and further conductivity increase (red). Eᵢₙₜ decay coincides in time with the end of the voltage-regulated plateau of Δψ_{PM}, which is shown as reference in time (dashed line). As shown in FIGS. 5B-E, local organelle transmembrane voltages are stimulated by several tens of millivolts, and these deviations from organelle resting potentials (hyper- and depolarized) provide the possibility of stimulating voltage-dependent channels.

FIG. 5A shows Eᵢₙₜ, taken in the center of the cell without any organelles present, as function of time for the exponential pulse (E⁰ₐₚₚ=1 kV/cm, τₚᵤₗₛₑ=40 μs). For comparison, Eᵢₙₜ resulting from a passive membrane (no EP, but with resting potential source) and the asymptotic EP model (no pore expansion) are given. A passive PM results in insignificant internal fields arising from membrane displacement currents. The active (EP) membrane response is strikingly different. The asymptotic EP model yields a partial redistribution of the equipotential voltage contours through the cell interior, and therefore Eᵢₙₜ reaches about 25% of E⁰ₐₚₚ. However, the SE model of EP with pore expansion leads to Eᵢₙₜ of almost 50% of E⁰ₐₚₚ. Thus in the present model, larger values of Eᵢₙₜ result from the non-linear increase of pore conductivity due to pore expansion. In contrast to claims that very large, nanosecond pulses are necessary, electric pulses with duration longer than τ_{PM} can achieve significant intracellular electric fields. Remarkably in FIG. 5A, the end of the voltage-regulated plateau in Δψ_{PM} at the PM pole coincides with the decay of Eᵢₙₜ. There is a narrowed time window when the cell interior experiences significant Eᵢₙₜ.

These intracellular fields are significant because first, they can cause transient changes of all organelle transmembrane voltages (FIGS. 5B-E), potentially leading to a response by voltage-sensitive organelle channels and pumps. Voltage-dependent calcium channels in the ERM, for example, are known to participate in the control of cytoplasmic Ca²⁺ concentration, which regulates cell processes such as secretion and gene transcription and has also been identified as a second-messenger signal for either apoptosis or necrosis. Thus changes in the intracellular Ca²⁺ concentrations are expected for conventional EP pulses, driven by release of intracellular stores through voltage-dependent ERM channels. Intra-organelle pH values are strongly regulated by many organelles to suit their individual biochemical functions, and transient changes can be caused by the perturbation of V-ATPase proton pumps. The voltage-dependent anion channel (VDAC) in the MOM allows for the transport of molecules below a size of 5 kDa. Furthermore, the mitochondrial permeability transition pore reacts to changes in the transmembrane voltage of the MIM and, if depolarized, may inhibit oxidative phosphorylation and the stimulation of ATP hydrolysis. Thus a range of unexplored possibilities exist for causing intracellular effects by changing organelle transmembrane voltages through conventional EP.

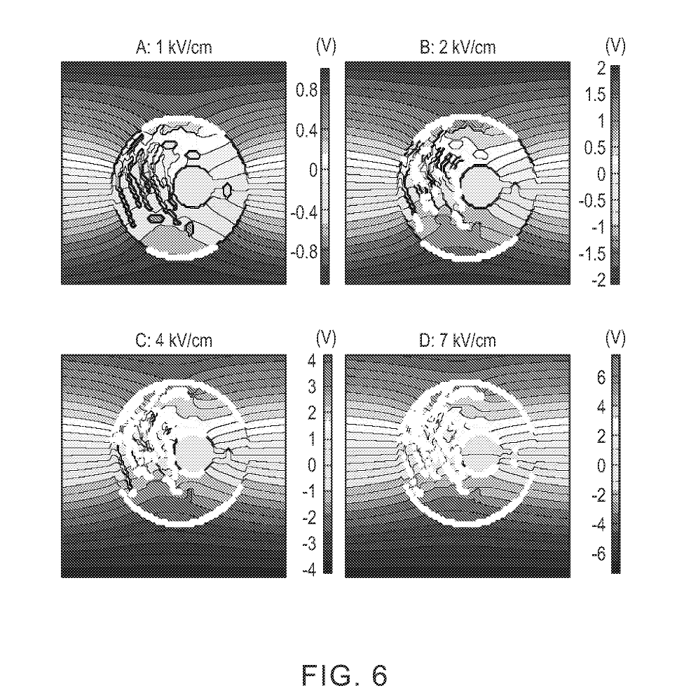

Second, as PM-EP leads to significant Ants pulse parameters may be found that lead to organelle EP. Experimental hints for intracellular EP by conventional pulses have been found with microelectrodes that use spatially focused inhomogeneous electric fields. Individual organelles such as mitochondria can be electroporated, but their smaller size requires higher applied field strengths. Indeed, organelle EP by conventional field pulses emerges at higher field amplitudes, as shown in FIG. 6. EP of the ERM is shown at 2 kV/cm, followed by the NM at 4 kV/cm, and the MOM at 7 kV/cm, opening new pathways for transport between cellular compartments. For example, nuclear EP provides an alternative direct path for nucleocytoplamic exchange of genetic material in addition to nuclear pore complexes, and may be relevant in optimized nucleofection protocols. In addition, the organelle membranes, if electroporated, become depolarized after the pulses. Organelle EP has been described for supra-EP by nanosecond field pulses, but is shown here to occur also for characteristic conventional EP pulses. The results thus suggest that conventional EP pulses may elicit intracellular effects, with some similar to those reported for nanosecond pulses. However, there is an essential difference between supra- and conventional organelle EP. The SE model of EP shows that pores expand insignificantly during nanosecond pulses. That is, they all remain small, whereas conventional pulses are sufficiently long for pores to expand to large radii. This has important consequences for molecular uptake and release and downstream cellular mechanisms, and will be further quantified herein.

FIG. 6 shows electric response of the cell system model to the exponential pulse (τₚᵤₗₛₑ=40 μs) for different field strengths at t=20 μs. PM and organelle EP, highlighted by white dots that correspond to local sites with at least one pore, emerge at higher field strengths (ERM at 2 kV/cm, NM at 4 kV/cm, and the MOM at 7 kV/cm). The boundary between the electroporated and non-electroporated region along the PM circumference depends on the field strength and expands towards the equitorial region for higher field strengths (also seen for the NM).

Electropermeability. The term electropeimeability Pₘ typically has been used as an entirely static quantity, without a molecular scale hypothesis and based on its traditional definition Pₘ=D_{S}K/dₘ that includes a static membrane partition function K, the solute's diffusion coefficient D_{S} and the membrane thickness dₘ=5 nm. The very nature of EP, however, calls for a dynamic definition that is based on electrically created and dynamic nanopores, and their transmembrane voltage-dependent size distribution. Accordingly, the local time-dependent electropermeability (units of meter per second)

$${P}_{m , i} ( {r}_{s} , t ) = \frac{π {D}_{s}}{{d}_{m}} {\int }_{{r}_{p , m i n}}^{{r}_{p , m ax}} {n}_{i} ( {r}_{p} , t ) {r}_{p}^{2} K ( {r}_{p} , {Δψ}_{PM , i} ( t ) ) H ( {r}_{p} , {r}_{s} ) d {r}_{p} \qquad ( 4 )$$

is shown for molecule/ion (solute “s”) of radius rₛ (specific molecular parameters are given in Table 1).

TABLE 1
Molecular Diffusion Parameters

| | Stokes | Diffusion coefficient | Electric |
|---|---|---|---|
| Molecule | radius [nm] | [m2/sec] | valence |
| Calcium | 0.16 | 1.6 × 10 − 9 | +2 |
| Trehalose | 0.43 | 0.57 × 10 − 9 | 0 |
| Propidium Iodide | 0.6 | 0.41 × 10 − 9 | +2 |
| Calcein | 0.6 | 0.41 × 10 − 9 | −4 |
| ATP | 0.77 | 0.3 × 10 − 9 | −4 |

The local electropermeability is a function of spatial position i, as the transmembrane voltage changes in time and space, and therefore the pore distributions and pore density vary with local membrane position. Permeability changes occur primarily through creation and subsequent expansion, shrinkage and decay of pores, and are thus controlled by the history of Δψ_{PM}, in turn governed by field strength, pulse duration, and pulse shape. The full dependence is condensed into the local pore distribution nᵢ(rₚ, t) that contains all information about the number of local pores, their sizes, and also their resealing dynamics. Inside a pore, each molecule experiences geometric hindrance H(rₚ, rₛ), which is related by both the geometric dimension of the molecule and the pore, and, if electrically charged, is subject to the voltage-dependent partition function K(rₚ,Δψ_{PM}(t)) based on electrostatic interaction of the molecule inside a pore. Expressions for the geometric hindrance H and the partition function are well known in the art. Their net effect is a well-known reduction of a solute's diffusion coefficient inside a pore. Therefore the electropermeability has to be quantified for each molecule individually.

With the local electropermeability P_{m,i} at hand, the cumulative number of transported molecules can be found from

$${N}_{s , PM} ( t ) = {A}_{cell} {\sum }_{i} {\int }_{0}^{t} {P}_{m , i} ( {r}_{s} , {t}^{′} ) \frac{{z}_{s} {υ}_{i}}{exp ( {z}_{s } {υ}_{i} ) - 1} ( \begin{matrix}{[ c ( {t}^{′} ) ]}_{i n} exp ( {z}_{s} {υ}_{i} ) - \\ {[ c ( {t}^{′} ) ]}_{out}\end{matrix} ) d {t}^{′} , \qquad ( 5 )$$

with the total cell membrane area A_{cell}, the solute's valence zₛ, the dimensionless local trans-membrane voltage vᵢ=eΔψ_{PM,i}/k_{B}T, and the solute concentration outside the cell [c(t)]ₒᵤₜ and inside the cell [c(t)]ᵢₙ. For the latter, stationary values are assumed, for simplicity. This approximation can be justified by the small total amount of molecules being transported, leaving the molecule's concentration in the media essentially unchanged, and the intracellular concentration still small. The sum over all local membrane sites i is shown in Equation (5). Equations (4) and (5) are solutions to the one-dimensional Nernst-Planck transport equation in a pore. As the transmembrane voltages have essentially opposite signs on the opposing hemispheres, symmetry arguments are employed to understand consequences for the electropermeability and actual molecular transport. Whereas P_{m,i}(rₛ,t is symmetric under sign reversal (Δψ_{PM,i}→Δψ_{PM,i}) and hence the electropermeability of the membrane is expected to be similar on both hemispheres, this is not the case for the number of transported molecules N_{s, PM}. Charged molecular transport depends on the sign of Δψ_{PM} and it's relation to present local molecular concentrations. Moreover, Equation (5) shows that electrophoretic transport and molecular diffusion are coupled. Separation is possible only in certain limiting cases.

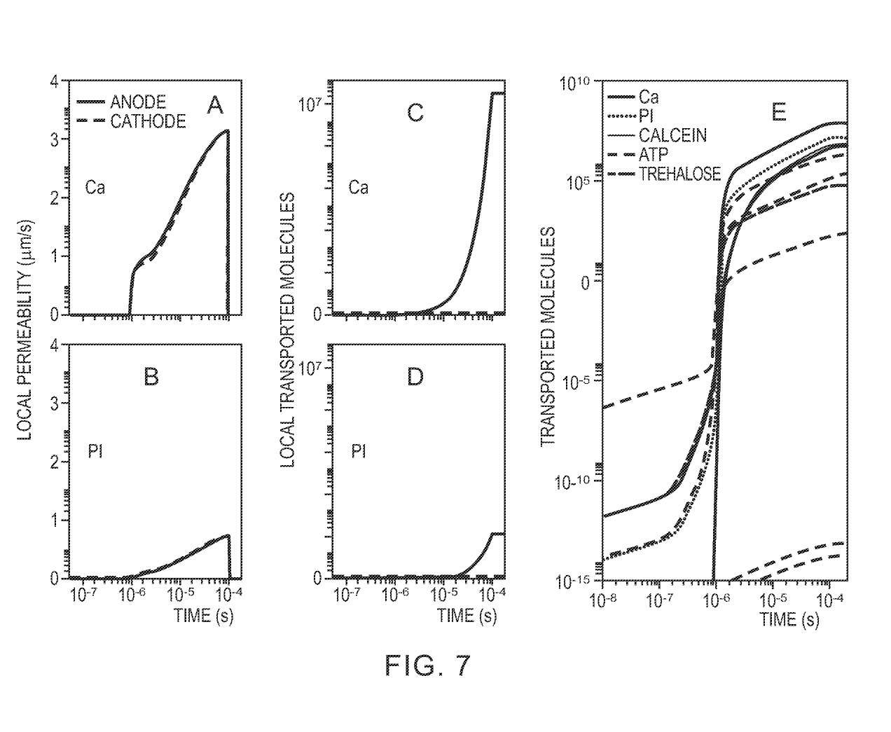

FIGS. 7A-E show electropermeability and number of transported molecules at the anodic and cathodic side as well as over the entire PM as function of time for trapezoidal-shaped conventional EP pulse (E⁰ₐₚₚ=95 kV/cm, 60 ns duration). FIGS. 7A-B show electropermeability for Ca and propidium iodide (PI). During the pulse (shaded area), there is an initial increase due to EP, followed by a second increase due to pore expansion. After the pulse, the PM electropermeability decays as the membrane discharges, even though the pore density persist for a much longer time scale. FIGS. 7C and D show anodic and cathodic Ca and PI transport. Whereas the electropermeability is essentially symmetric at the anode and cathode (FIGS. 7A-B), transport during the pulse is driven electrophoretically through the anodic side only. FIG. 7E shows total PM transport. Solid curves show the SE model of EP prediction, dashed curves are the corresponding result for the asymptotic EP model without pore expansion. Before the pulse start, insignificant molecular transport occurs through equilibrium pores. During the pulse the electroporated part of the PM is significantly permeable to the molecules in Table 1, hence transport occurs in detectable numbers. Pore expansion is essential to allow for the transport of the larger molecules, as can be judged by the difference to the asymptotic EP model.

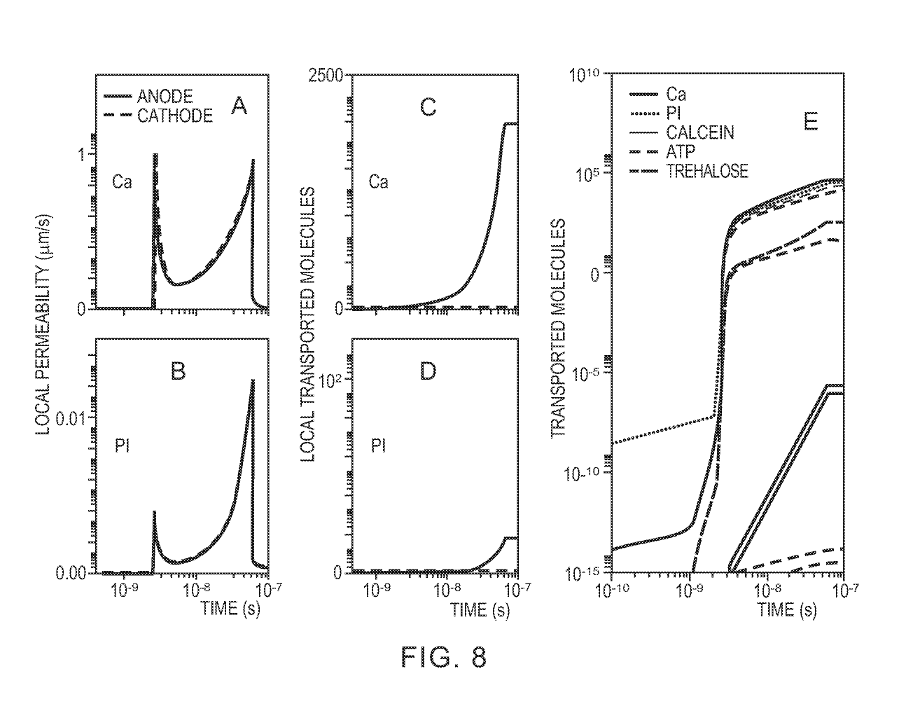

FIG. 8A-E show permeability and number of transported molecules at the anodic and cathodic side as well as over the entire PM as function of time for trapezoidal supra-EP pulse (E⁰ₐₚₚ=1 kV/cm, 100 μs duration). FIG. 8A-B show anodic and cathodic electropermeability for Ca and PI. During the pulse (shaded area), there is an initial increase due EP followed by a second increase that reflects the start of pore expansion. After the pulse, the electropermeability decays as the membrane discharges, although the pore density persists on a much longer time scale. FIGS. 8C-D show anodic and cathodic Ca and PI transport. Whereas the electropermeability is essentially symmetric at the anode and cathode, transport during the pulse is driven electrophoretically through the anodic side only. FIG. 8E show total transport. Solid curves show the SE model of EP prediction, dashed curves are the corresponding result for the asymptotic EP model without pore expansion. Before the pulse start, insignificant molecular transport occurs through equilibrium pores. While the pore density is two orders of magnitude higher than in FIG. 7, the electropermeability remains small (but not zero) for larger molecules and hence the number of transported molecules such as PI, Calcein, and ATP is much lower; possibly below routine detection limits. For nanosecond pulses, pore expansion appears relatively unimportant.

FIGS. 7 and 8 compare the anodic and cathodic electropermeabilities P_{m,i}(rₛ,t) and the amount of molecular transport for trapezoidal-shaped pulses for conventional EP (E⁰ₐₚₚ=1 kV/cm, 100 μs duration, 1 μs rise and fall times) and supra-EP (E⁰ₐₚₚ=95 kV/cm, 60 ns duration, 1 ns rise and fall times). As examples, Ca²⁺, calcein and PI can be used as traditional fluorescent probes that indicate the occurrence of EP, as well as trehalose and ATP. The conventional pulse reaches a pore density 1×10¹⁴ m⁻². FIGS. 7A-B show a sharp rise of the electropermeability for Ca and PI due to EP and subsequent pore expansion, and a reduction of P_{m,i}(rₛ, t) after the pulse even though the pore density persists for the mean pore lifetime τₚ. This is due to the discharged PM having essentially zero transmembrane potential (Δψ_{PM}→0), leading to pore shrinkage and a reduction of local values of the partition function K. The associated number of transported Ca²⁺ and PI molecules at the anodic and cathodic side as well as for the total PM is shown in FIGS. 7C-E. For convenience of comparison, a concentration difference of 100 μM is assumed for all species. Although the specific cellular preparation is very likely different from this choice, it is attempted here to demonstrate the differences in transport that result from the interplay of geometry and electrical conditions. The temporal behavior of molecular transport initially exhibits a non-linear increase due to pore creation and expansion and then, after REB, a steady linear increase at elevated transmembrane potentials. At the end of the pulse the number of accumulated molecules flattens, as the electropermeability is strongly reduced. Strikingly, the anodic and cathodic P_{m,i} (rₛ, t) are similar, but the molecular transport occurs entirely through the anodic side because of the sign-dependence of electrophoresis. All considered molecules traverse the cell membrane in numbers (>10⁵) that may be detected with a suitable measurement device. The comparison with the asymptotic model of EP in FIG. 7E (dashed lines) demonstrates the influence of pore expansion on molecular transport. In particular, highly-charged molecules (ATP, calcein) would essentially not traverse the membrane without pore expansion. Trehalose is an example of an electrically neutral molecule that has biotechnological relevance to cell preservation. As neutral molecules are not subject to Born energy penalties inside the pore, and hence the partition function is K=1, the permeability before the pulse is actually higher than for example Ca, despite the larger size of trehalose. But electrophoresis is not acting, and thus all other molecules are transported in higher numbers.

To compare with supra-EP conditions, FIGS. 8A-B show the electropelmeability and FIGS. 8C-D the number of transported molecules over the anodic and cathodic PM side. The pore density reaches 4×10¹⁶ m⁻², markedly higher than for the conventional EP situation discussed in FIG. 7. But more pores do not translate into higher membrane permeability or more transport. Instead, during this pulse, the increase of the PM electropelmeability occurs due to combined interplay of creating many small pores and elevated transmembrane potentials, but the electropermeability for the molecules of Table 1 remains smaller than for conventional EP, as these pores are all remain small. Only the permeability to the smaller ions (Na⁺, K⁺, Cl⁻) is actually larger, and forms the basis for holding down the voltage during supra-EP conditions. After the pulse, similar to FIG. 8A, the pore density persists for the mean pore lifetime τₚ, but due to the discharged PM with an essentially zero transmembrane potential, the electropermeability is also reduced, resulting in the flattening with time of the transported molecules. Again, the electropelmeability is essentially symmetric on both sides of the PM, but transport occurs dominantly over the anodic side. The number of molecules traversing the membrane is much smaller than for conventional EP, and the transport of larger molecules is insignificant. It does not mean that those larger molecules are excluded per se, but the actual number of transported molecules per single pulse may be below the detection limit of the experimental physical apparatus. Here, the number of transported PI is much smaller than 10⁵, in accordance with the reported observation that PI does not to enter the PM immediately after the pulse, and only a train of many pulse repetitions or a secondary biological effect long after the pulse may eventually show hints of PI transport. The comparison with the asymptotic model of EP shows, again, that pore expansion appears negligible on the nanosecond time scale, as the differences between the asymptotic and the SE models of EP are much less significant than on the longer time scale of conventional EP.

An important observation presented is the unavoidable presence of intracellular electric fields during conventional EP. Several consequences have been presented here. Thus, the PM barrier to successful manipulation of signal transduction mechanisms is surpassed and the biochemical nature of organelles may be controlled and manipulated by conventional EP.

Using this model, the uptake or release of certain molecules may be examined for their potential role in irreversible EP induced cell death by necrosis. Most interesting here is the ATP behavior. As seen in FIG. 8A, ATP can be released during conventional EP conditions in detectable numbers, but ATP it is essentially confined to the cell interior in supra-EP conditions. The sudden loss of cellular ATP has been correlated with necrotic cell death. Thus the ATP-efflux may contribute to irreversible EP by necrotic cell death in the case of conventional EP, and the lack of ATP-efflux for supra-EP conditions may support the observed apoptotic cell death.

The terms “electropermeability” and “electropermeabilization” have been used qualitatively in the art, mostly to describe the ability of a particular molecule to traverse a membrane after an electric field was applied. Equation (4) is a quantitative representation of electropermeability that is based on the EP mechanism. Nanosecond pulses involve two to three orders of magnitude more PM pores, but because of limited pore size, the uptake of traditionally used membrane integrity dyes (e.g., PI) and release of ATP is restricted, whereas conventional (longer, smaller) EP pulses allow for substantial molecular transport. Therefore, nanosecond pulses create pores which are not large enough to release death molecules from the mitochondria.

It has been argued before that electropermeability is asymmetric as molecules were observed to enter the cell preferentially through one hemisphere. However, transport of molecules does not necessarily represent a degree of permeability, as forces may hinder molecular transport even if the membrane is permeable. In the case presented here, the electropermeability is practically the same on both poles (remaining small differences result from the resting potential) but transport occurs primarily through the anodic (cathodic) side as Δψ_{PM} drives the positively (negatively) charged molecules in (out) the cell, see FIGS. 7 and 8.

### Electropermeability

Neutral Molecules. For electrically neutral molecules with zero residual charge (z=0) like trehalose, sucrose, and other sugars, the partition function is K(rₚ, Δψ_{PM})=1 (neglecting dipole interactions), thus no restriction for transport, and the corresponding local membrane electropermeability reduces to

$${P}_{m , i} ( {r}_{s} , t ) = \frac{π {D}_{s}}{{d}_{m}} {\int }_{{r}_{p , m i n}}^{{r}_{p , m ax}} n ( {r}_{p} , t ) {r}_{p}^{2} H ( {r}_{p} , {r}_{s} ) d {r}_{p} . \qquad ( 6 )$$

The membrane permeability still depends on Δψ_{PM} via the pore distribution n(rₚ,t), but not explicitly. Yet the molecule may remain excluded from the pore interior by geometric hindrance. Molecular transport is governed by concentration differences (Fick's law), and the number of accumulated molecules is

$${N}_{s , PM} ( t ) = {A}_{cell} {\sum }_{i} {\int }_{0}^{t} {P}_{m , i} ( {r}_{s} , {t}^{′} ) ( {[ c ( {t}^{′} ) ]}_{i n} - {[ c ( {t}^{′} ) ]}_{out} ) d {t}^{′} , \qquad ( 7 )$$

This is a good approximation unless Pₘ(rₛ,t) becomes so large that diffusion in bulk media is limiting.

Zero transmembrane voltage. The case of zero transmembrane voltage, Δψ_{PM}=0, which may occur for some time after the pulse due to total discharge of the membrane (before the membrane pores decay and the resting potential sources are reestablished), the partition function can be expanded and yields

$$K ( {r}_{p} , {Δψ}_{PM} = 0 ) = \frac{{w}_{0} {z}^{2}}{2 n ( 1 + exp ( {w}_{0} {z}^{2} ) ( {w}_{0} {z}^{2} - 1 ) ) - exp ( {w}_{0} {z}^{2} ) {w}_{0} {z}^{2}} . \qquad ( 8 )$$

Hence charged molecules still experience a Born energy wₒ(rₚ) inside the pore and the partition function is further dependent on geometrical factors of the pore, given here by n, the relative size of the entrance region of the pore. The resulting molecular transport

$${N}_{s , PM} ( t ) = {A}_{cell} {\sum }_{i} {\int }_{0}^{t} {P}_{m , i} ( {r}_{s} , {t}^{′} ) ( {[ c ( {t}^{′} ) ]}_{i n} - {[ c ( {t}^{′} ) ]}_{out} ) d {t}^{′} . \qquad ( 9 )$$

again is dominated by concentration differences (Fick's law) across the membrane.

Zero Born energy. The limit of vanishing Born energy, w₀(rₚ)=0, which may be reached for sufficiently large membrane pores, the partition function is reduced to

$${lim}_{{w}_{0} -> 0} K ( {r}_{p} , {Δψ}_{PM} ) = 1. \qquad ( 10 )$$

Also for large pores, the hindrance factor H=1, but the two limits are reached at different pore radii. The local electropermeability then is

$${P}_{m , i} ( {r}_{s} , t ) = \frac{π {D}_{s}}{{d}_{m}} {\int }_{{r}_{p , m i n}}^{{r}_{p , m ax}} n ( {r}_{p} ) {r}_{p}^{2} d {r}_{p} . \qquad ( 11 )$$

The resulting molecular transport is given by

$${N}_{s , PM} ( t ) = {A}_{cell} {\sum }_{i} {\int }_{0}^{t} {P}_{m , i} ( {r}_{s} , {t}^{′} ) \frac{{z}_{s} {υ}_{i}}{exp ( {z}_{s} {υ}_{i} ) - 1} ( \begin{matrix}{[ c ( {t}^{′} ) ]}_{i n} exp ( z {υ}_{i} ) - \\ {[ c ( {t}^{′} ) ]}_{out}\end{matrix} ) d {t}^{′} . \qquad ( 12 )$$

There is an apparent asymmetry between the cell's anodic and cathodic sides in terms of molecular transport: Whereas the electropermeability is strictly symmetric in the sign of Δψ_{PM}, the total transport depends on the sign of Δψ_{PM} and it's relation to present local concentrations.

Drift-dominated transport. In the case of equal concentrations on each side of the membrane [c(t)]i=[c(t)]ₒ=[c(t)], Equation (5) becomes

$${N}_{s , PM} ( t ) = {A}_{cell} {\sum }_{i} {\int }_{0}^{t} {P}_{m , i} ( {r}_{s} , {t}^{′} ) [ c ( {t}^{′} ) ] z {υ}_{i} d {t}^{′} , \qquad ( 13 )$$

with the electropermeability given in Equation (4). This special case resembles a drift-dominated situation that is linear in Δψ_{PM} (Ohm's law), but a non-linear dependence on Δψ_{PM} remains in the partition function K and thus the electropermeability. In all other cases, N_{s,PM} exhibits an interdependence between the solute's concentration differences and voltage differences across the membrane, hence a coupling between drift and diffusion.

### Example 2

### Activation of Apoptosis by Electrical Release of Death Molecules from Mitochondria

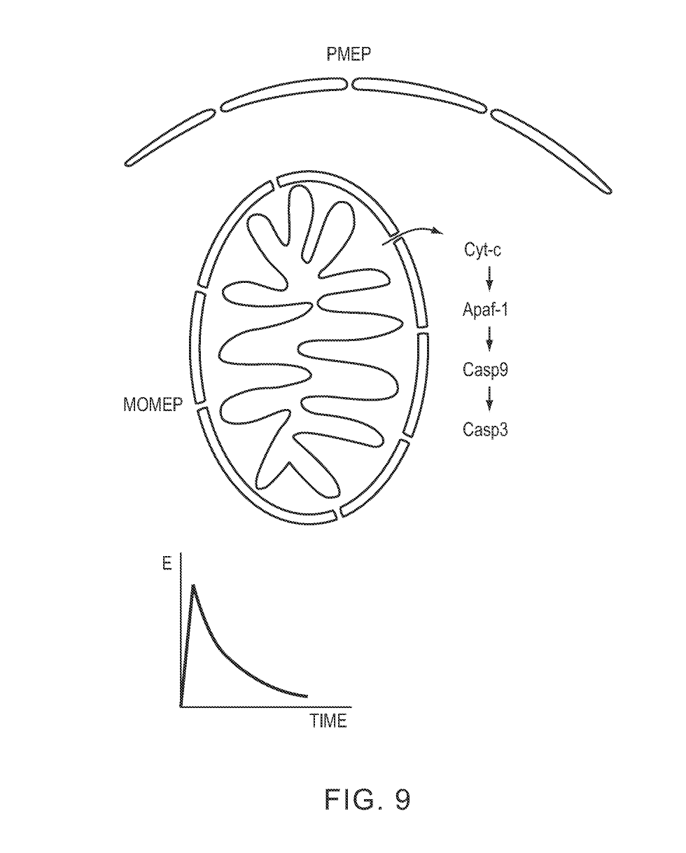

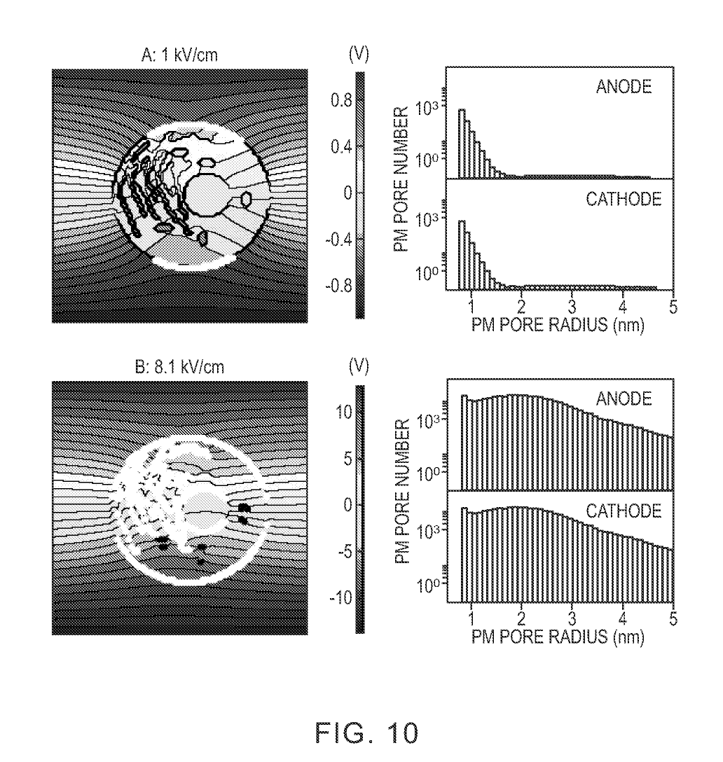

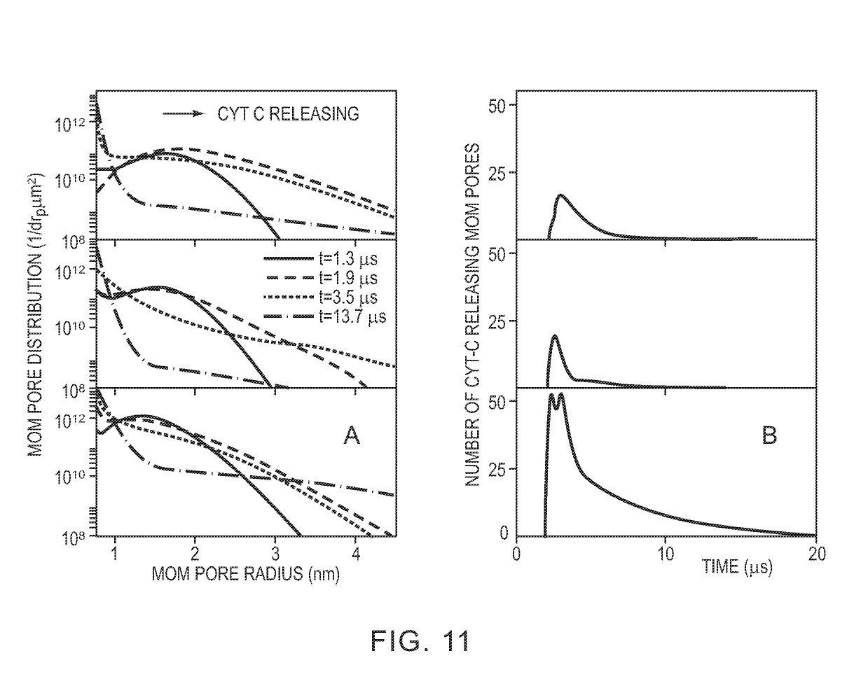

Mathematical modeling shows that appropriate electrical pulses can directly create transient phospholipid MOM pores by electroporation (MOMEP), producing a nearly instantaneous, physically driven MOM permeabilization (MOMP) and therefore bypassing the biochemical signaling cascade. The mechanism quantitatively describing dynamic pore distributions that allow sufficient cyt-c release and irreversible caspase activation for experimental conditions that cause electrically induced apoptosis is shown. A direct physical method to induce apoptosis by electrical MOMP (FIG. 9) is shown.

FIG. 9 shows apoptosis by MOMEP. Strong electric field pulses, by electroporating the plasma membrane (PMEP), cause large intracellular electric fields that can lead to organelle EP such as MOMEP. Electrophoretic cyt-c efflux through transient phospholipid MOM pores activates caspases and the mitochondrial apoptosis pathway.

Conventional EP causes a transient permeabilization of the PM that allows the transfer of DNA, RNA, proteins, drugs, and nanoparticles into cells. Examples include DNA delivery, gene therapy, and electrochemotherapy of solid tumors. Conventional EP involves pulses with characteristic times (duration, rise and fall times) exceeding the PM charging time τPM (typically 0.1 to 1 μs for mammalian cells in suspension). For pulses too small for EP, the PM is polarized for times exceeding τPM, and the internal electric field is insignificant. However, this passive characterization neglects structural membrane reorganizations associated with EP, a general mechanism by which mammalian cells respond to fields greater than 300 V/cm. As shown here, electric fields enter the cell interior by conventional EP waveforms, may permeabilize organelles, and in that way lead to new possibilities for intracellular interventions. Virtually every EP application has the goal to transfect a foreign molecule into the cellular compartment. A different strategy is proposed: Applying EP to transport endogeneous molecules across a cellular membrane to achieve a biologic effect.

In an early experiment, exponential pulses Eₐₚₚ(t)=E⁰ₐₚₚexp[−t/τₚᵤₗₛₑ] with time constant τₚᵤₗₛₑ=40 μs and field strength E⁰ₐₚₚ=8.1 kV/cm were applied to Jurkat T-lymphoblasts and HL60 cells, and the induction of apoptosis was observed. The underlying mechanism, however, remained unknown. When the same pulse is used to examine the response of the cell system model, the resulting pore sizes at the PM, and the emergence of organelle EP (FIG. 10). Electric field penetration arises by PMEP, with pore expansion controlling the magnitude of the intracellular field. Most PM pores have radii less than 2 nm, but a pore subpopulation grows during the pulse and is essential for the delivery of macromolecules into cells. The dynamic pore distribution depends on the local transmembrane voltage, which itself depends on the pore distribution via a non-Ohmic pore conductance, and thus creating a highly-nonlinear system with interactions coupled through electrolytes to all membranes. Strikingly, fields enter cell organelles for a field strength of 8.1 kV/cm. This is the hallmark of organelle EP, which has recently been described for supra-EP by nanosecond field pulses. But on the nanosecond time scale those transient pores expand insignificantly, and the larger pores of conventional EP are essential for the death molecule release discussed here. An adiabatic temperature rise estimate is about 4 K for the 8.1 kV/cm pulse, consistent with a non-thermal effect.

FIG. 10 shows electric response, pore sizes, and the emergence of organelle EP. The cell system model's response to exponential pulse with 1 kV/cm (FIG. 10A) and 8 kV/cm (FIG. 10B) at t=30 μs is shown. Equipotentials (black lines) show the partial redistribution of the electric field by the PM and it's dependence on EP. PM and organelle EP are highlighted by white (for clarity, MOMEP sites are black) dots that correspond to local membrane sites with more than one pore, equivalent to a pore density of more than 10¹³ m⁻². Pore creation, expansion and shrinkage determine the reversible high conductance membrane state associated with EP. Organelle EP emerges at high field strengths. PM pore-size histograms show the number of pores with a specific size. Most pores remain small (r₀<2 nm), but a subpopulation of pores expands to larger sizes during the pulse and is responsible for macromolecular uptake and release. The pore-size distribution has a small asymmetry between the anodic and cathodic PM hemispheres caused by both the PM resting potential and the presence of cell organelles.

FIG. 11 shows MOMEP and cyt-c release. FIG. 11A shows MOM pore distributions for three individual mitochondria show heterogeneous dynamic behavior. Significant pore sizes develop during the pulse with sufficiently large MOM pores to release cyt-c. In FIG. 11B, the number of cyt-c releasing pores N_{MOM}^{cyt-c}(t) are shown for the same mitochondria as in FIG. 11A.

A main conclusion of the analysis is that the MOM is transiently permeabilized by the electric field, and death molecules are released. For a quantitative understanding, the MOM pore distributions n_{MOM}(rₚ, t) of the three individual mitochondria that are electroporated by the 8.1 kV/cm pulse (FIG. 11A) are considered. Conventional EP pore distributions are highly dynamic. Here their heterogeneity depends on the mitochondria's orientation with respect to Eₐₚₚ, and also on field distortions by nearby organelles, notably the ER. Thus the number of pores and their size differ locally. The number of MOM pores N_{MOM}^{cyt-c} that have sufficient size to release cyt-c, a roughly spherical 14 kDa protein with radius r_{cyt-c} of 1.5-1.75 nm, as

$${N}_{MOM}^{cyt - c} ( t ) = {\int }_{{r}_{cyt - c}}^{{r}_{m ax}} {n}_{MOM} ( {r}_{p} , t ) d {r}_{p} . \qquad ( 1 )$$

The maximum pore radius used for the present field condition is rₘₐₓ=10 nm. FIG. 11B shows N_{MOM}^{cyt-c} corresponding to the individual MOM pore distributions in FIG. 11A. Overall the model predicts 20-50 transient MOM pores per mitochondria that are large enough to release cyt-c. About half the pores will have the local field in the extracting direction, such that cyt-c is released by electrophoresis during the pulse. MOMP by MOMEP thus leads to large enough phospholipid pores to release cyt-c.

The number of cyt-c molecules released by MOMEP must be sufficient to cause downstream activation of apoptosis. Cyt-c release can be reversible if only few molecules are released. The amount of cyt-c required to achieve a switch-like caspase-3 activation depends on the cell type, and the key is the amount of apaf-1 molecules activated by cyt-c. Intracellular nM concentration of apaf-1, with field-independent constraints on the amount of IAP present, leads to irreversible caspase activation and then cell apoptosis. To estimate of the number of cyt-c molecules released from all mitochondria in the cell model, first it is necessary to calculate Nₚₒᵣₑ^{cyt-c}, the number of cyt-c molecules released through a single pore. From Ampere's and Ohm's laws have, respectively:

$${N}_{pore}^{cyt - c} = \frac{{I}_{pore}^{cyt - c} {t}_{open}}{{qz}_{cyt - c}} \\ {I}_{pore}^{cyt - c} = {\frac{π {r}_{p}^{2} {F}^{2} {Δψ}_{m}^{MOM} {D}_{cyt - c} {z}_{cyt - c}^{2}}{{RTd}_{m}} [ cyt - c ]}_{i} , \qquad ( 2 )$$

with the cyt-c diffusion coefficient D_{cyt}_{_{—}}_{c}=6×10⁻¹⁰ m²/s and the electric valence z_{cyt}_{_{—}}_{c}=+8. The free cyt-c concentration in the intermembrane space [cyt-c]ᵢ=1 mM is assumed to be 20% of the total cyt-c concentration. Interactions with cardiolipin do bind a fraction of the cyt-c to the mitochondrial inner membrane (MM), but the electric field is expected to disrupt this weak electrostatic binding. With typical values for the MOM transmembrane voltage during the pulse of Δψₘ^{MOM=}1 V, a participating MOM pore radius of rₚ=2 nm, and tₒₚₑₙ=10 μs, Nₚₒᵣₑ^{cyt-c}=3×10³ released through a single pore were found. For an average of 10 pores in a single mitochondria with the external field in the extracting direction, the estimated cytosolic cyt-c release leads to [cyt-c]_{cell}=6 μM. Cells may have more mitochondria than considered in the model cell system (see Methods), and accordingly more cyt-c will be released. The affinity of cyt-c to apaf-1 depends on the cytosol ionic strength. For typical potassium concentration (150 mM), the cyt-c to apaf-1 association constant is 4×10⁷ M⁻¹, and about 650 cyt-c molecules are needed to activate a single apaf-1. Thus MOMEP leads to an activated apaf-1 concentration of about [apaf-1]=9 nM, which should be sufficient to irreversibly activate caspases.

Two experimental features provide further support for the mechanism of apoptosis by MOMEP. First, the ionic strength of the medium is critical for, i.e., a lower conductivity extracellular medium did not cause apoptosis at the same field strengths, even though the PM is electroporated. This occurs because spatially distributed voltage division results in a smaller EP-induced conductance change at the PM, a significantly reduced intracellular field, and thereby suppression of MOMEP. Second, apoptosis induction was blocked in the experiment by the caspase inhibitor zVAD. Thus caspases were activated as a result of the field exposure, and cyt-c release by MOMEP provides a mechanistic explanation for this hitherto unexplained observation. Further, the MIM remains intact for these fields, as MIMEP would occur for either higher field strengths and much longer time scales, so that ATP generation is initially sustained by the mitochondrial transmembrane potential (Δψₘ^{MIM}) to drive the apoptotic pathway, rather than switching to necrotic cell death observed at low concentrations of ATP.

The release of other death molecules by MOMEP requires larger MOM pore radii. For example, the IAP-neutralizing Smac/DIABLO (100 kDa) and Omi/HtrA2 (50 kDa) exceed the molecular mass of cyt-c. Caspase-independent cell death pathways, particularly important in tumors where the caspase activated apoptotic pathway is blocked, are related to the release of AIF (35 kDa) and endo-G (67 kDa). A scaling relation rₚₒᵣₑ∝M^{1/3} between the molecular size and mass, based on Stokes law, suggests that approximately twice the cyt-c pore size is needed to release Smac/DIABLO. For the present electrical exposure only 1 MOM pore in the model system was found with that size, but an order of magnitude more for a 40 μs trapezoidal pulse of the same field strength.

The description of MOMEP in a highly interacting environment with simultaneous plasma and other organelle membrane EP is an example of the increasing capability of in silico cell models for quantitative biology. MOMP by MOMEP provides a mechanistic basis of apoptosis induction by electric fields, and has the potential to guide further hypothesis-driven experimental research that will optimize death molecule release. In particular, the release of larger death proteins is expected to be increased substantially by either somewhat larger and/or longer pulses or pulse trains, all without significant heating. The release of proteins from the mitochondrial intermembrane space into the cytosol by MOMEP is also a special case of a new intervention: rapid, electrically controlled delivery of almost any size molecules between intracellular compartments.

### Methods

Modular, multiscale transport lattices in biology. The transport lattice (TL) of the cylindrical cell system model is based on local charge transport, storage, sinks, and sources rather than field equations. Nanometer-scale membrane modules represent conductive phospholipid regions, resting potential sources, and local electrical capacitances. The local EP membrane model is included via subcircuits interacting within the TL at respective membrane sites. These modules are assigned to local membrane areas and connected to their nearest micrometer-scale electrolyte neighbors to form a Cartesian TL. Basic features of the TL method are described elsewhere, with all model parameters used here are given in the references. TLs have ˜2×10⁴ interconnected local models and are solved by Kirchhoff's laws, using Berkeley SPICE version 3f5 (University of California, Berkeley, Calif.).

System model for the cell with organelles. Referring again to FIG. 1A, FIG. 1A shows a cross-sectional geometry and system model of cell (radius 10 μm) with organelles (ERM, mitochondria, and nucleus); indicating direction of external electric field. FIG. 1B shows local circuit models for electrolyte (open rectangle) and membrane (filled rectangle) assembled in different configurations (bottom two rows of the panel). FIG. 1C shows an equivalent circuit representation of the dynamic EP model that is solved at all local membrane sites. The current source I_{N} is the pore creation and destruction term in Equation (4). The effective voltage on each capacitor CN represents the pore distribution n(rₚ, t) for different pore radii (not a physical voltage in the system model. Pore radius drift and diffusion are related to the current source I_{N} and the resistor R_{N}, respectively. The local pore distribution in subcircuit (FIG. 1C) determines the pore conductivity and hence the membrane current Iₘ as input in Mₘ (FIG. 1B).

The geometry is shown in FIG. 1A and includes the PM (10 μm radius), nuclear membrane (NM; 3 μm radius), the endoplasmic reticulum membrane (ERM) and, in close proximity to the ERM, five mitochondria (approximated with 1 μm×2 μm), each with an outer membrane (MOM), inner membrane (MIM), and intermembrane space of 15 nm. Invaginated christae are represented by an increased effective MIM area that is a factor f_{A}=5 larger than the MOM. The system depth is d_{sys}5=0.35 μm, from which the cellular volume V_{cell}=d_{sys}πr_{cell}² follows. Thus the density of 5 mitochondria in the cylindrical cell volume corresponds to about 190 mitochondria in a spherical cell with the PM radius. The dielectric constant of the extra- and intracellular electrolytes is 80. Close to a pore the membrane is treated as pure lipid and assigned a dielectric constant of 2.1. In contrast, average PM and organelle membrane capacitance have a dielectric constant of 5 because of protein contributions. Membranes have a passive conductance; lower values for NM and MOM account for their leaky nature. A simplified resting potential source model is used to represent approximately the effect of ion pumps and channels in creating a membrane resting potential Δψ_{m,rest}(−90 mV, 90 mV, and −200 mV for PM, ERM, and MIM, respectively; Δψ_{m,rest} for NM and MOM are zero). Voltages applied at planar virtual electrodes at the top and bottom of the system boundaries provide the uniform applied field.

Dynamic EP model The biophysical mechanism of EP is hypothesized to involve transient aqueous pore creation, destruction, expansion and contraction as described by the Smoluchowski equation (SE) for the time-dependent pore distribution n(rₚ, t)

$$\frac{\partial n}{\partial t} = \frac{\partial }{\partial {r}_{p}} [ {D}_{p} ( \frac{\partial n}{\partial {r}_{p}} + \frac{n}{{k}_{B} T} \frac{\partial W}{\partial {r}_{p}} ) ] . \qquad ( 3 )$$

Here Dₚ is the diffusion constant in pore radius space, the pore radius is rₚ and the pore energy W(rₚ, Δψₘ(t)) contains a mechanical and an electrical, Δψₘ(t)-dependent part. Pore formation is plausibly described by a rate equation model over many temporal orders of magnitude. Here, the number of pores N₀=n(r₀) drₚ with minimum size pore radius r₀=0.8 nm follows from

$$\frac{\partial {N}_{0} ( {r}_{0} , t )}{\partial t} = A {e}^{B {Δψ}_{m}^{2} / {k}_{B} T} - \frac{{N}_{0} ( {r}_{0} , t )}{{τ}_{p}} .$$

Reported pore lifetimes vary from milliseconds to minutes or longer. The illustrative value of τₚ=3 ms, as well as the parameters A and B, are based on experimental data.

An equivalent circuit representation accounts for SE pore dynamics (FIG. 1C). The first term of Equation (3) describes diffusion in pore radius space and is represented by resistor R_{N}=(drₚ)²/Dₚ between succeeding pore radii r and r+drₚ, with drₚ=0.05 nm the spacing of the natpore space discretization. The drift-term in Equation (3) is implemented by an active current element

$${I}_{r , r + {dr}_{p}} = \frac{{D}_{p}}{2 {k}_{B} {T ( {dr}_{p} )}^{2}} [ \begin{matrix}n ( {r}_{p} + {dr}_{p} ) + \\ n ( {r}_{p} )\end{matrix} ] [ \begin{matrix}W ( {r}_{p} + {dr}_{p} ) - \\ W ( {r}_{p} )\end{matrix} ] . \qquad ( 5 )$$

The expression ∂n(rₚ, t)/∂t|drift=Iᵣ_{ₚ}_{-dr}_{ₚ}_{,r}_{ₚ}−Iᵣ_{ₚ}_{,r}_{ₚ}_{+dr}_{ₚ} contains both the linear and the quadratic energy derivatives of the SE. The unit capacitor at each pore node is CN=1, and the current source 'N represents the rate of change of N0, as given by the r.h.s. of Equation (4). The dynamic EP circuit is solved at every local membrane site (area Aₘ=0.12 μm²), from which the pore conductivity is obtained and hence the total pore current′m at the PM and all organelle membranes 20 as input into the membrane module Mm. Thus the EP circuit interacts locally with the distributed physical TL.

The expression ∂n(rₚ, t)/∂t=Iᵣ_{ₚ}_{-dr}_{ₚ}_{,r}−I_{r,r+dr}_{ₚ} contains both the linear and the quadratic energy derivative terms of the SE. The dynamic EP model is solved at every local membrane site (local area Aₘ=0.12 μm²).

### Example 3

### Screening Waveforms In Silico for Mitochondria Membrane Electroporation-Induced Apoptosis

A transport lattice (TL) method can be used to generate equivalent cell system models with two, three, or more cells in two dimensions (2D). These models can be easily extended to comprise many cells of a tissue, and in particular cells of different sizes. These models can also be combined with large scale tissue models to determine an intracellular electric condition in large scale tissue models and then to combine these intracellular electric conditions with cell system to obtain the number of death molecules released due to a particular electric field waveform. This method thus provides a means to generate a plurality of different waveforms, with different shape, duration, and field strength, and to select a particular waveform that leads to a specific intracellular electric field, which for example, leads to a specific number of released death molecules, for example cytochrome-c, to induce apoptosis in the cell.

In addition to the single (isolated) cell model described previously in Example 1, the two and three cell models with dynamic pores at the MOM (OMM) of each mitochondrion and the asymptotic model for EP at the PM and MIM (MIM) were also used. The rationale for using the dynamic EP model only for the MOM (OMM) of each mitochondrion is the present practical limitation of computer power and the previous finding that for the conductive transport of small ions that dominate electrical behavior, the asymptotic model gives a reasonable approximation. Moreover, this approximation is provides conservative estimates. If the dynamic EP model is used in the PM the intracellular electric field is larger (not smaller) than if the asymptotic EP model is used (see FIG. 5 of Example 1). This means that even more large pores would be created in the MOM if the dynamic EP model could be used in the PM of the cells of the two and three cell models.

The two-cell system model consists of a 60 μm×60 μm×0.5 μm volume. The two cells have radii of 10 μm and 20 μm, and both have depth 0.5 μm. Each cell contains two mitochondria, with each mitochondrion 2 μm×1 μm×0.5 μm. As described previously each mitochondrion has an outer membrane (OMM or MOM) with a resting potential of zero, and a mitochondrial inner membrane (IMM or MIM) with a resting potential source and local resistance such that the resting potential is −200 mV.

Electroporation within a local membrane area l×l is represented by the asymptotic EP model for the PM and IMM (MIM), as in some previous models, but the OMM (MOM) has a dynamic pore model based on the SE assigned to it, to test for the evolution of transient aqueous pores large enough to transport cyt-c from the space between the OMM and IMM into the cytoplasm.

If sufficient numbers of death molecules such as cytochrome-c, second mitochondria-derived activator (Smac, also termed DIABLO), apoptosis-inducing factor (AIF), EndoG and Omi/HtrA2, a cascade leading to apoptosis is unleashed. An example of quantitative modeling of release cyt-c by OMM (MOM) EP is shown. Other, larger death molecules should be released via larger transient phospholipid-based pores than those that release cyt-c.

The three-cell system model also has a system volume of 60 μm×60 μm×0.5 μm. To obtain the three cell model a third cell with radius 5 μm was added to the two cell model, so that a characteristic linear dimension, the cell radius, varies by a factor of four within the cell system model. Each cell also contains two mitochondria, with each mitochondrion 2 μm×1 μm×0.5 μm.

Solutions to each system model result in electric potentials and transmembrane voltages, and also the number of transient aqueous pores in each local membrane area (l×l=0.225 μm²). In the case of the dynamic local EP model based on the SE, the pore size distribution in each local area is obtained. The black lines or curves are equipotentials. More closely spaced equipotentials correspond to larger electric fields, which are perpendicular to the equipotentials. White regions indicate regions with electroporation. The waveforms generally cause reversible electroporation, not irreversible electroporation.

The dynamic pore distribution in the OMM (MOM) is of particular interest. The present estimates are based on transport of released molecules through transient aqueous pores as the transport rate limiting step. In order to release cyt-c (radius of about 1.5 nm), pores with radii ≧1.5 nm are needed. This criterion includes the concept that with a net charge (valence) of z_{cyt}=+8, the positively charged cyt-c is favored to enter pores with some phospholipids along the pore interior surface having negative (attractive) charge. The fluctuating transient aqueous pores are also expected to slightly expand transiently as the local electric field within the entrance, interior and exit regions of the pore acts to force cyt-c through the pore. This reasoning underlies the approximate criterion of seeking pulse waveforms that create at least 10 pores with r≧1.5 nm for at least 10 μs, or the related criterion of requiring r≧2 nm for at least 10 μs.

This means that a larger number of pores r≧1.5 nm for a shorter time should also release sufficient cyt-c to cause apoptosis. For example, if 33 or more pores exist with r≧1.5 nm for 3 μs sufficient cyt-c should be released. A more general approximate criterion is the condition n_{>1.5}×tₚₒᵣₑ=100 pore μs. Here n_{>1.5} denotes the number of pores with radii greater than or equal to 1.5 nm and tₚₒᵣₑ denotes the time that these pores are present.

As the geometric and electric parameters of the death molecules are known, from the number of pores and the size of the pores in the mitochondria that are created by an electric pulse waveform, the number of any death molecule released may be calculated.

Using different pulse waveforms, the model's physical response (redistributed fields via equipotentials, membrane potentials, dynamic pore population evolution), and then biochemical changes (amounts of cyt-c release, activation of caspase-3 that usually forces apoptosis) are examined.

The present two- and three-cell system models have for each cell a PM and two mitochondria with IMM and OMM as in the above single cell model. All membranes have spatially distributed resting potential sources that create appropriate resting potentials. Specifically Δψ_{MOM}=0, Δψ_{PM}=−90 mV, and Δψ_{MIM}=−200 mV.

EP creates local dynamic pore distributions (pore distributions, n(r), that depends on time and the transmembrane voltage-dependent at each local area of a membrane in the cell system models. This causes a spatially varying membrane shunt conductance that decreases Δψ, which is often close to zero for a time governed by the pore lifetime, here taken to be 3 ms, taken from measurements on lipid bilayer membranes.

The use of sources of electrical stimuli, suitable electrodes and means for placement of electrodes are briefly described, which can be used with the in silico (computer modeling) results regarding electric field waveforms in practical situations such as in vivo treatment of living tissue.

### Results and Discussion

Results from individual pulse waveforms (pulse shape and magnitude) are obtained from two and three cell system models. The purpose of these first in silico pulse waveform screening modeling results is to examine specific waveforms for their ability to produce pore populations that evolve a sufficient number of OMM (MOM) pores. As noted in Methods, the present criterion is an approximate criterion, viz. n_{>0.5}×tₚₒᵣₑ=100 pore μs. According to the above criterion, if a sufficient number of pores evolve to maintain or exceed 1.5 nm radii (pore size necessary to release cyt-c), if the product of the number of such pores is present long enough, then sufficient cyt-c is released to cause apoptosis. It should be emphasized that other pulses that evolve somewhat larger pores will release Smac (DIABLO) and other death molecules simultaneously, and that these additional death molecules can overcome inhibitions that can inhibit apoptosis if Smac were not also released.

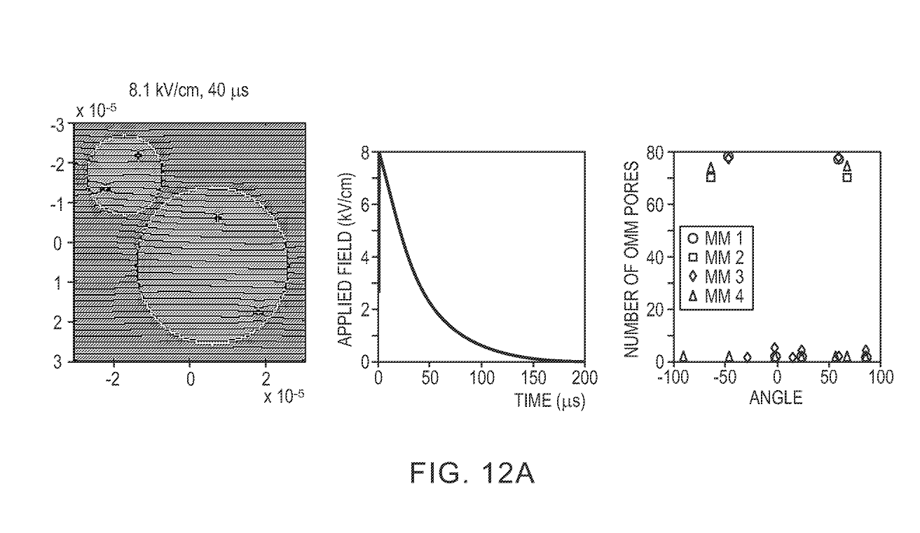

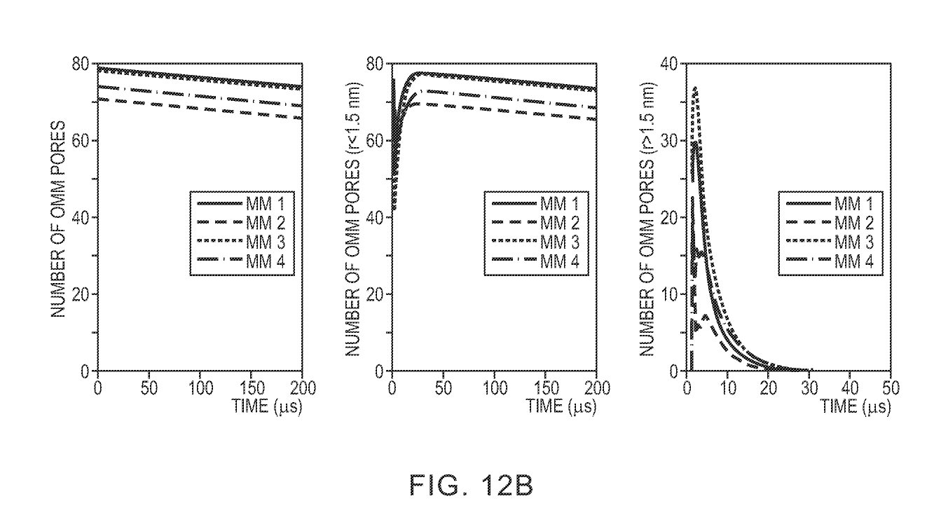

8.1 kV/cm, 40 μs exponential pulse: This is an acceptable waveform for making most cells apoptotic (see FIG. 12). FIG. 12 shows a 8.1 kV/cm, 40 μs exponential pulse. The two circular cells are 10 μm and 20 μm in radius. In 2D, the four mitochondria are 2 μm×1 μm in size. The 121×121 geometry represents 60 μm×60 μm×0.5 μm simulation volume. EP models: asymptotic model for PM and IMM; SE model for OMM.

The electric field pulse waveform is a so-called exponential pulse which has an initial linear rise from zero to a peak value of 8.1 kV/cm in a rise time of 1 μs, followed by an exponential decay in time (t) with an decay time constant of τpulse=40 μs. The single electric field pulse was applied with direction from the top to the bottom of the two cell system model. The model's description of the “Number of OMM Pores (r≧1.5 nm)” shows four curves that depict this number of cyt-c transporting pores. By visual inspection MM3 peaks at 37 pores, MM1 peaks at 30 pores, MM4 peaks at 15 pores and MM2 at 7 pores. All but MM2 satisfy the approximate cyt-c release criterion based on a 1.5 nm pore size lasting ≧10 μs. The approximate temperature rise is 5.5° C.

One OMM (MOM; here MM1) does not satisfy this particular criterion for all cells becoming apoptotic. In the present model the intra- and extra-cellular medium conductivities are the same. If, however, the cell interior medium were assigned a somewhat higher electrical resistivity (lower conductivity) then the intracellular electric field would be larger, and more extensive poration would occur. An effective higher intracellular medium would result if more organelles could be included in the models, but this requires more computational power than presently available. With this additional interpretation, however, this waveform is consistent with the prediction that most cells will be made apoptotic, as only MM1 has too few pores with radii ≧1 μm for at least 10 μs.

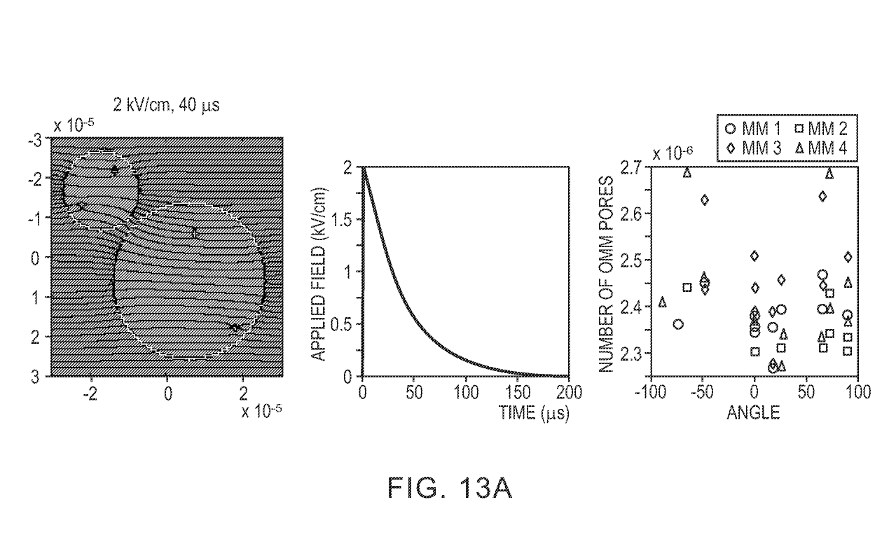

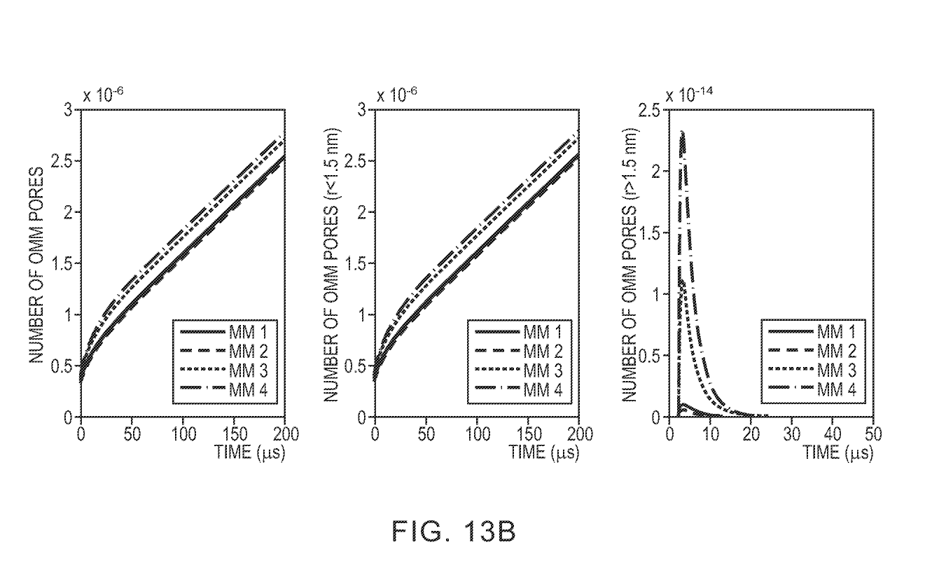

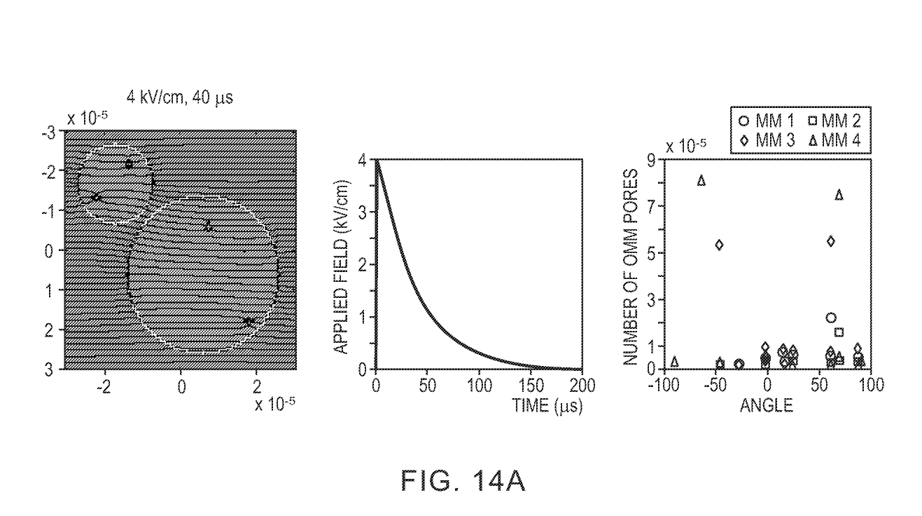

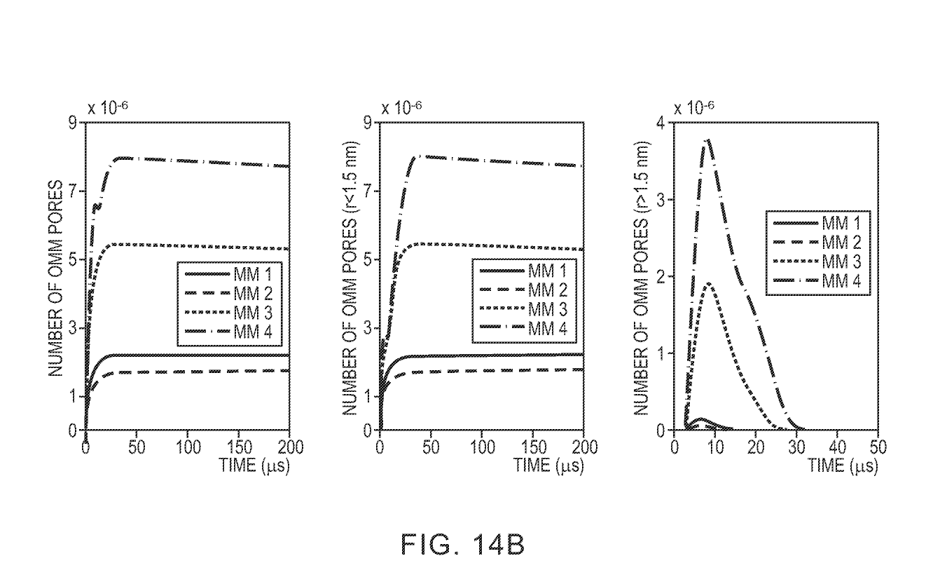

2 kV/cm, 40 μs exponential pulse: This waveform males negligible numbers of cells apoptotic (see FIG. 13). FIG. 13 shows a 2 kV/cm, 40 μs exponential pulse. The two circular cells are 10 μm and 20 μm in radius. In 2D, the four mitochondria are 2 μm×1 μm in size. The 121×121 geometry represents 60 μm×60 μm×0.5 μm simulation volume. EP models: asymptotic model for PM and IMM; SE model for OMM. All four cyt-c releasing pore curves are extremely small (the maximum is ˜2.3×10⁻¹⁴ pores. Although the approximate temperature rise is only ˜1° C., this electric field waveform evolves too few cyt-c transporting pores.

4 kV/cm, 40 μs exponential pulse: This waveform makes negligible numbers of cells apoptotic (see FIG. 14). FIG. 14 shows a 4 kV/cm, 40 μs exponential pulse. The two circular cells are 10 μm and 20 μm in radius. In 2D, the four mitochondria are 2 μm×1 μm in size. The 121×121 geometry represents 60 μm×60 μm×0.5 μm simulation volume. EP models: asymptotic model for PM and IMM; SE model for OMM. All four cyt-c releasing pore curves are again extremely small (the maximum is ˜3.8×10⁻⁶ pores. Although the approximate temperature rise is ˜2.5° C., this electric field waveform also evolves too few cyt-c transporting pores.

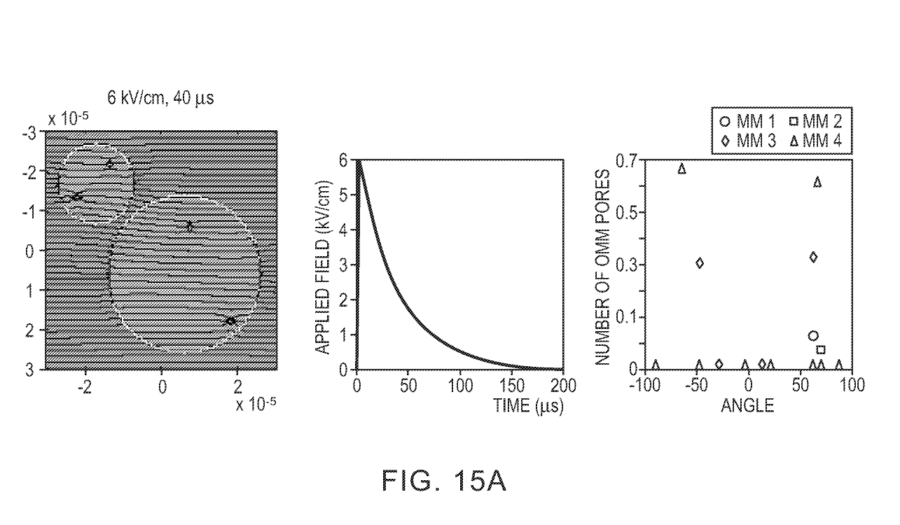

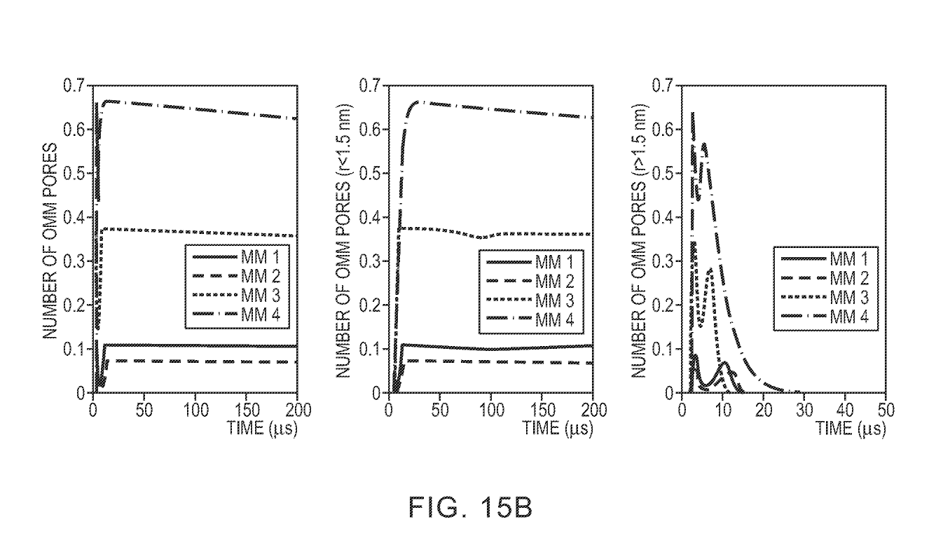

6 kV/cm, 40 μs exponential pulse: This waveform makes too few cells apoptotic (see FIG. 15). FIG. 15 shows a 6 kV/cm, 40 μs exponential pulse. The two circular cells are 10 μm and 20 μm in radius. In 2D, the four mitochondria are 2 μm×1 μm in size. The 121×121 geometry represents 60 μm×60 μm×0.5 μm simulation volume. EP models: asymptotic model for PM and IMM; SE model for OMM. Although the number of cyt-c releasing pores in MM1-MM4 have pore number curves that are much larger than the 2 and 4 kV/cm results above, the pore numbers peak in the range 0.1 to 0.7, i.e. slightly less than (the maximum is 0.64 pores). The curves exhibit somewhat complicated behavior, which illustrates the complex nature of the many electrical interactions with this two cell model. This is consistent with the idea that a simple description of cyt-c releasing pores is unlikely to be possible. Although the approximate temperature rise is ˜4° C., by the present criterion this electric field waveform will male very few cells apoptotic.

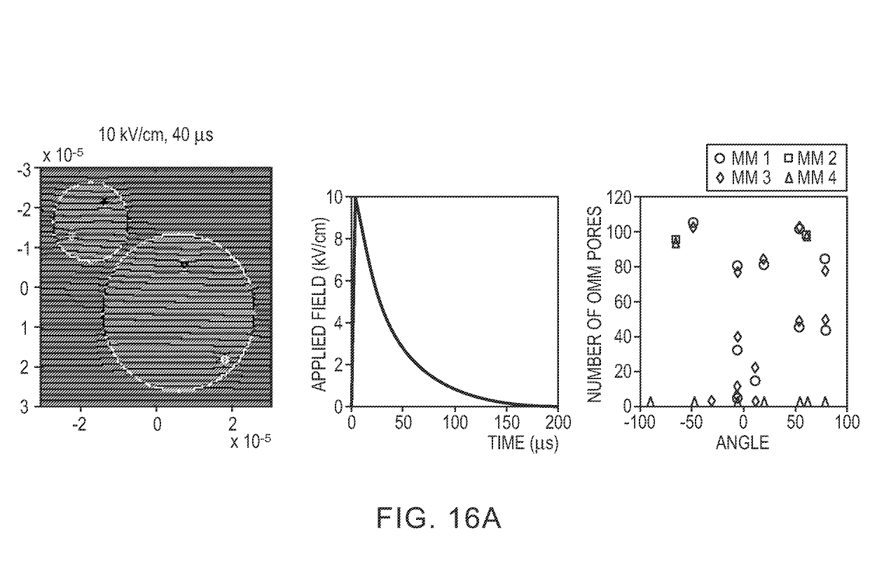

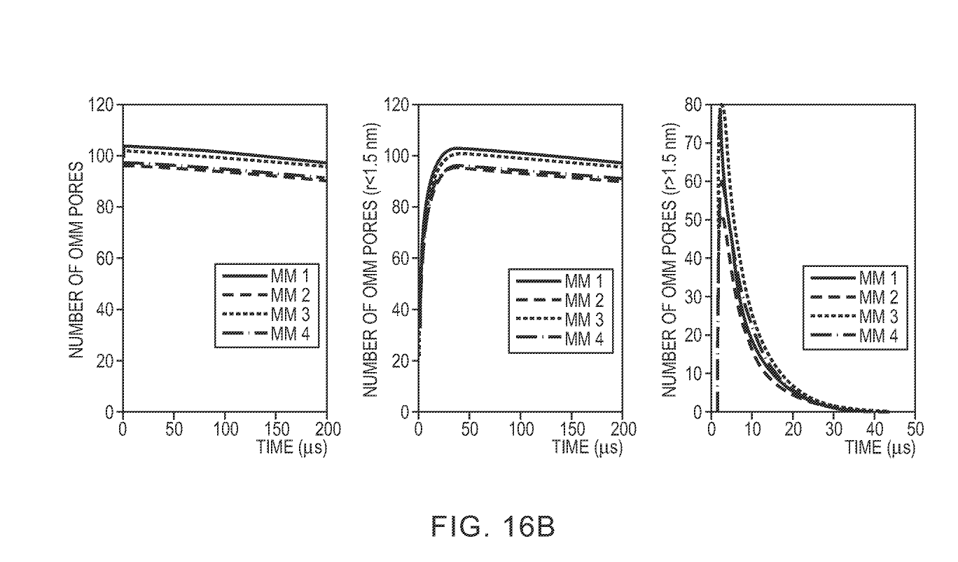

10 kV/cm, 40 μs exponential pulse: This waveform makes all cells apoptotic (see FIG. 16). FIG. 16 shows a 10 kV/cm, 40 μs exponential pulse. The two circular cells are 10 μm and 20 μm in radius. In 2D, the four mitochondria are 2 μm×1 μm in size. The 121×121 geometry represents 60 μm×60 μm×0.5 μm simulation volume. EP models: asymptotic model for PM and IMM; SE model for OMM. By visual inspection all four OMM (MOM; MM1-MM4) reach values well above the approximate criterion for cyt-c release, and should therefore drive all cells into apoptosis. The approximate temperature rise is ˜7° C. For deep tissue well from the body's surface a normal core temperature is 37° C. A brief rise to 7+37 is 42° C. For significant thermal damage to occur it is generally believed that the temperature, T(t), must exceed 42° C. for a long time, as biological repair mechanisms are expected to prevent significant accumulation of thermal damage at 42° C. or less. For peripheral tissue, particularly skin, the initial temperature is much lower, e.g. ˜26° C. Skin-associated tissues are likely candidates for the multiple session embodiment in which time is provided for phagocytes to migrate to the treated tissue volume with apoptotic cells, and these tissues normally have lower temperatures in the range 25 to 30° C., so larger temperature rises can be accepted before there are any concerns regarding thermal damage.

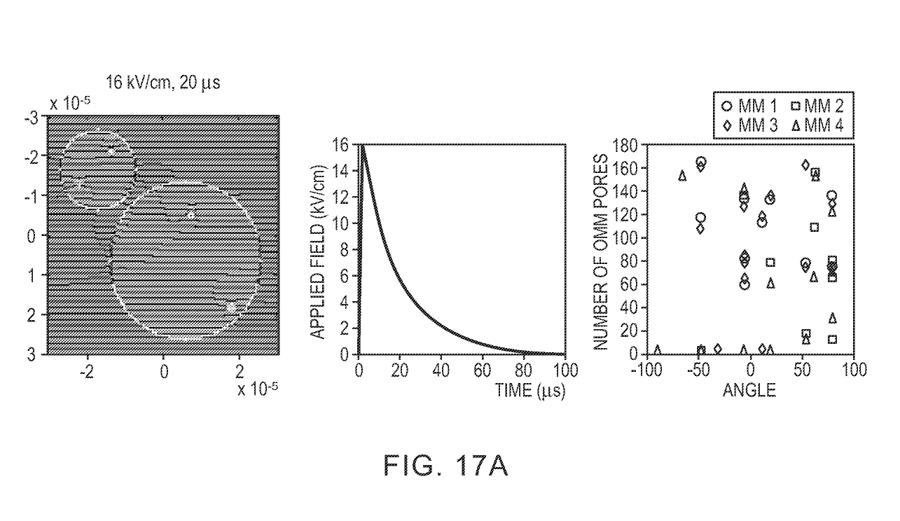

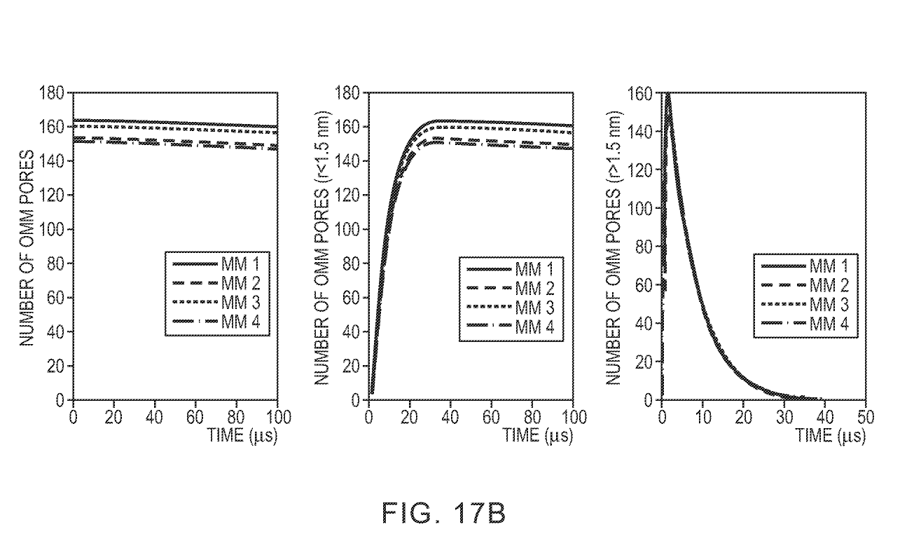

16 kV/cm, 40 as exponential pulse: A shortened version of this waveform will make all cells apoptotic (see FIG. 17). FIG. 17 shows a 16 kV/cm, 20 μs exponential pulse. The two circular cells are 10 μm and 20 μm in radius. In 2D, the four mitochondlia are 2 μm×1 μm in size. The 121×121 geometry represents 60 μm×60 μm×0.5 μm simulation volume. EP models: asymptotic model for PM and IMM; SE model for OMM.

Specifically, a 16 kV/cm, 3 μs exponential pulse will male all cells apoptotic. By visual inspection all four OMM (MOM; MM1-MM4) reach values well above the approximate criterion for cyt-c release. The approximate temperature rise is more than 10° C. if the indicated exponential pulse decay time constant of 20 μs is retained. However, if the same magnitude of 16 kV/cm is used with a significantly shorter exponential pulse decay time constant of 3 μs is used, then the temperature rise is only ˜6° C. Further, all of the pore populations consisting of pores with radii ≧1.5 nm exhibit a depletion rate that is approximately an exponential decay time constant of about 10 μs, which indicates that the pore population contracts pores faster than the electric field decreases. This is complicated, non-intuitive behavior. Thus, a pulse that rose and fell quickly, e.g. a 1 μs rise time and a 3 μs fall time will evolve almost as many cyt-c releasing pores as the 20 μs electric field pulse waveform used in the present screening test. Within the first 3 μs of the 20 μs waveform there are at least 120 cyt-c releasing pores. This number of pores with radii exceeding 1.5 nm is significantly more that the 33 pores need by the approximate criterion. The temperature rise for a 16 kV/cm, 3 μs pulse wave form is about 5.5° C. The analysis thus concludes from this screening test that a 16 kV/cm, 3 μs exponential pulse waveform will drive all cells into apoptosis with an acceptable temperature rise.

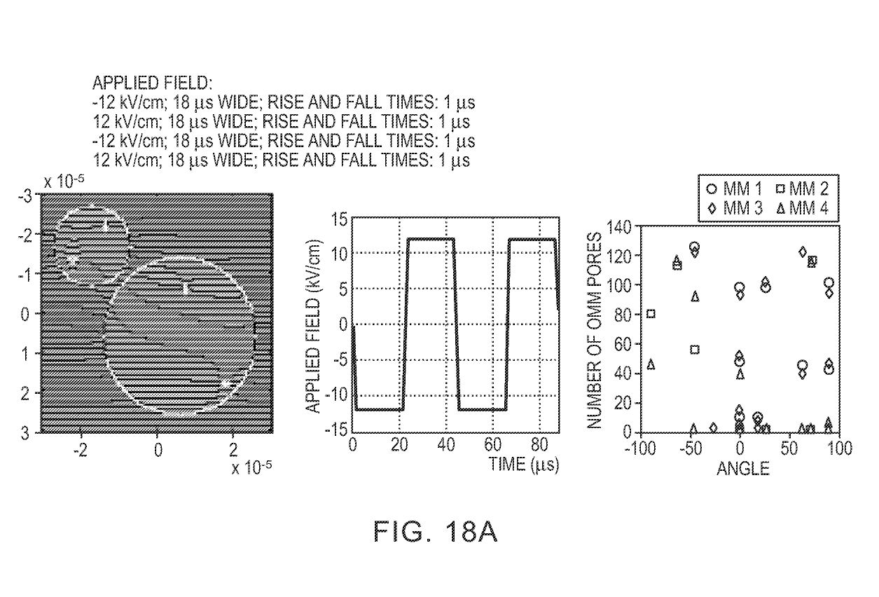

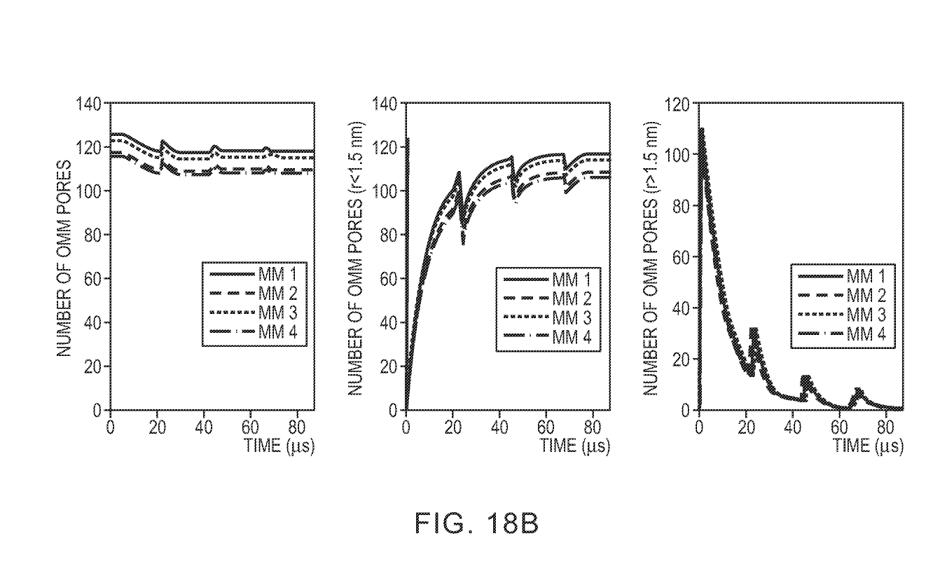

12 kV/cm, 18 μs bipolar pulse (part of the screened bipolar waveform): This waveform makes all cells apoptotic (see FIG. 18). FIG. 18 shows a 12 kV/cm, 18 μs bipolar pulse. The two circular cells are 10 μm and 20 μm in radius. In 2D, the four mitochondria are 2 μm×1 μm in size. The 121×121 geometry represents 60 μm×60 μm×0.5 μm simulation volume. EP models: asymptotic model for PM and IMM; SE model for OMM.

Specifically, the initial 12 kV/cm, 10 μs negative-going portion makes all cells apoptotic. This set of screening tests also involves the two cell model, but uses alternating polarity (bipolar) trapezoidal pulses. At first glance the complete two cycle bipolar trapezoidal waveform is unacceptable, because the total “on time” is about 80 μs, and for the complete two cycles of negative-going and then positive-going applied field the temperature rise is much too large. However, a single trapezoidal pulse of duration 10 μs can be considered, as the pulse could be terminated at that time. By examining the plot of the number of cyt-c releasing >1.5 nm in the first 10 μs of the pulse, it is seen that all 4 OMM (MOM; MM1-MM4) evolve cyt-c releasing pores such that the number of such pores in each outer mitochondlial membrane exceeds ˜50 pores at the end of 10 ms. Pulse waveform polarity is unimportant, because both the EP model and heating have Δψ² dependence, and the effect of either a negative or positive pulse is approximately the same. To make this approximate statement it is argued that a slight asymmetry in the EP response due to the resting potential source is very small. The temperature rise is ˜5° C. Thus, a trapezoidal waveform of magnitude 12 kV/cm and duration 10 μs is predicted to drive all cells in the two cell system into apoptosis based on the criterion that pores with radii≧1.5 μm for ≧10 μs.

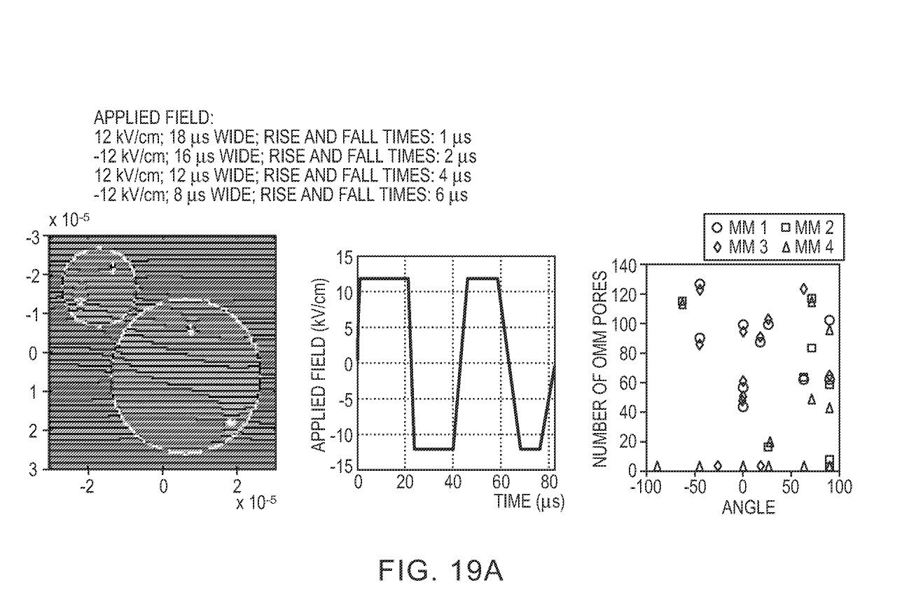

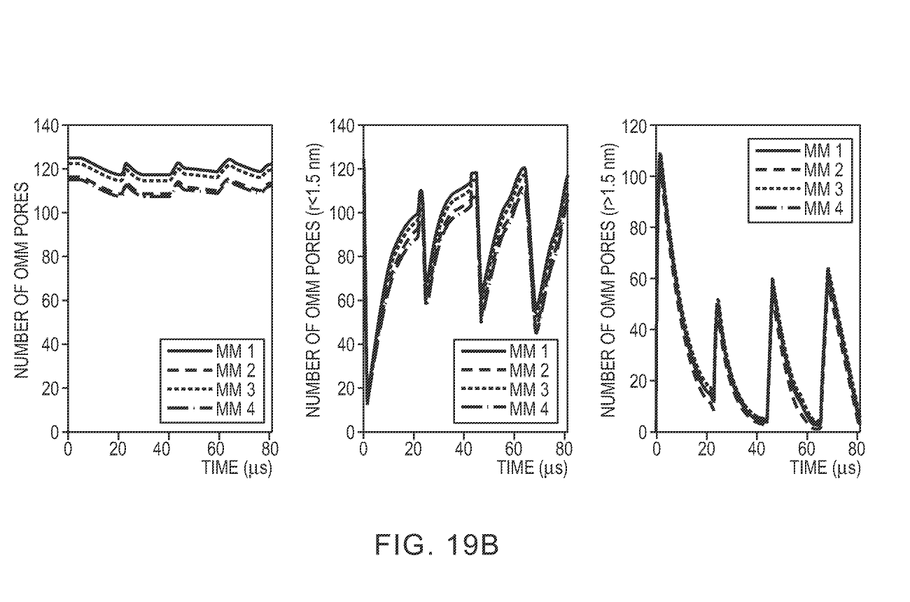

12 kV/cm, 18 μs bipolar pulse (part of the screened bipolar waveform): This waveform makes all cells apoptotic (see FIG. 19). FIG. 19 shows a 12 kV/cm, 10 μs bipolar pulse. The two circular cells are 10 μm and 20 μm in radius. In 2D, the four mitochondria are 2 μm×1 μm in size. The 121×121 geometry represents 60 μm×60 μm×0.5 μm simulation volume. EP models: asymptotic model for PM and IMM; SE model for OMM.

Specifically, the initial 12 kV/cm, 10 μs positive-going portion males all cells apoptotic. The main difference between this electric field pulse waveform in the first 10 μs and the above waveform labeled 12 kV/cm, 10 μs bipolar pulse is the pulse polarity. Specifically, the above waveform goes negative, while the present pulse goes positive. A slight dependence of the asymmetric two cell model on polarity is expected, because of the non-zero resting potential sources (and corresponding resting potentials) in the PM and IMM (MIM) of each cell. Thus, the cyt-c releasing pore numbers (60 or larger in the first 10 μs) are slightly different than those above bipolar waveform, but are well above the 10 pores of the approximate criterion. The temperature rise during the first 10 μs is also 5° C. Thus, an initially positive-going trapezoidal waveform of magnitude 12 kV/cm and duration 10 μs will also drive all cells apoptotic.

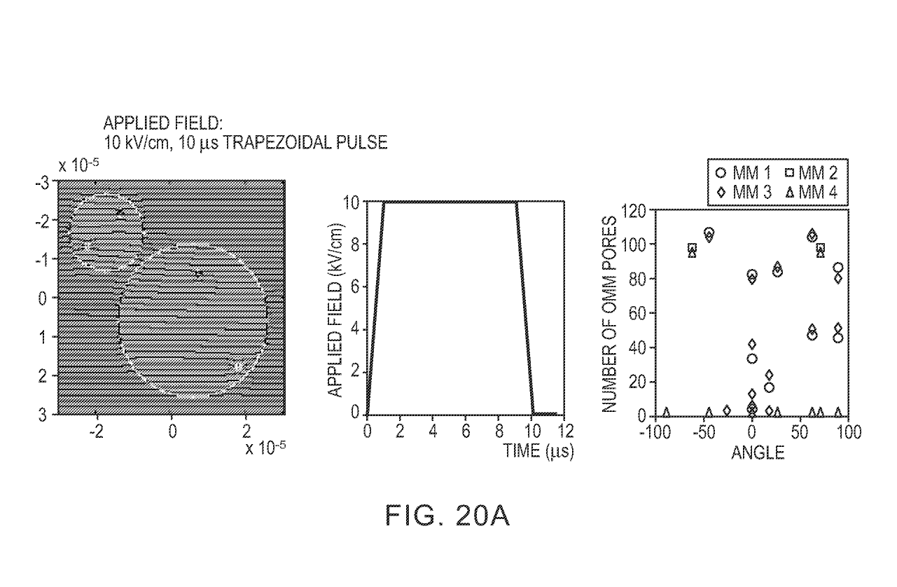

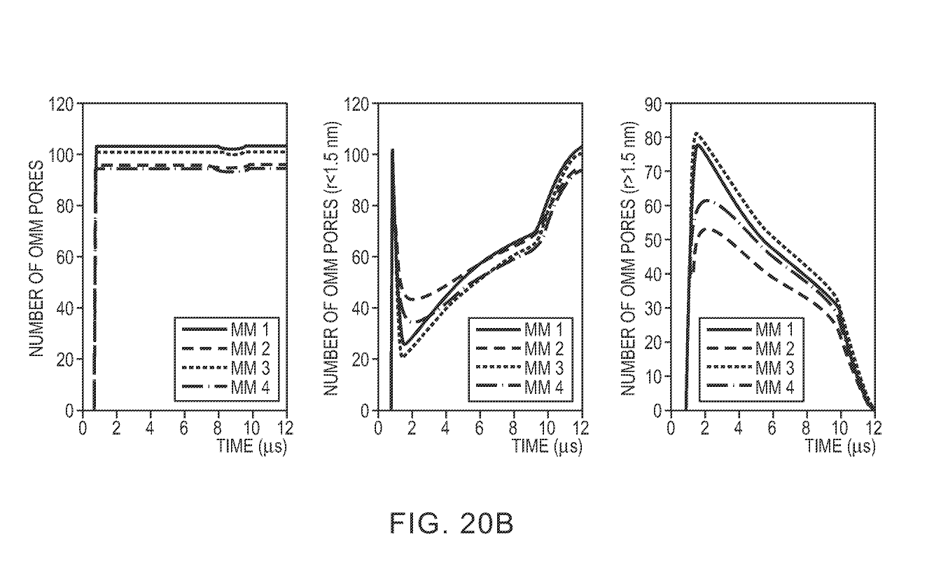

10 kV/cm, 10 μs trapezoidal pulse: This is an acceptable trapezoidal waveform for making all cells apoptotic (see FIG. 20). FIG. 20 shows a 10 kV/cm, 10 μs trapezoidal pulse. The two circular cells are 10 μm and 20 μm in radius. In 2D, the four mitochondlia are 2 μm×1 μm in size. The 121×121 geometry represents 60 μm×60 μm×0.5 μm simulation volume. EP models: asymptotic model for PM and IMM; SE model for OMM. The plot of curves labeled “Number of OMM pores (r<1.5 nm))”—that is, OMM pores with a radius of less than 1.5 nm—has a time axis from zero to 12 μs. For the time interval 1 to 10 μs MM1-MM4 have significantly more than 30 pores with radii ≧1.5 nm) during this 9 μs time interval. By the present criterion the models need about 11 such pores in each MM (MOM). The temperature rise for this pulse waveform is about 4.5° C. Thus, this in silico screening test predicts that this electric field waveform will make all cells apoptotic.

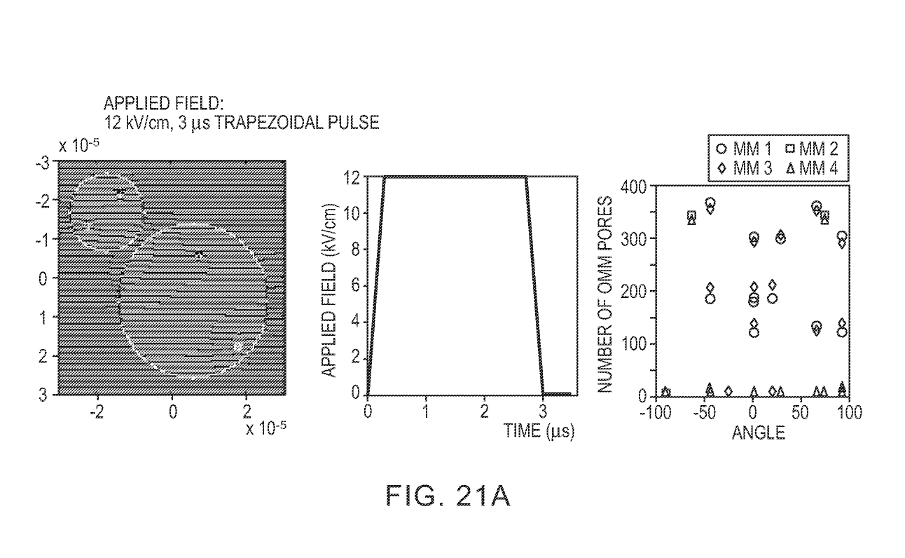

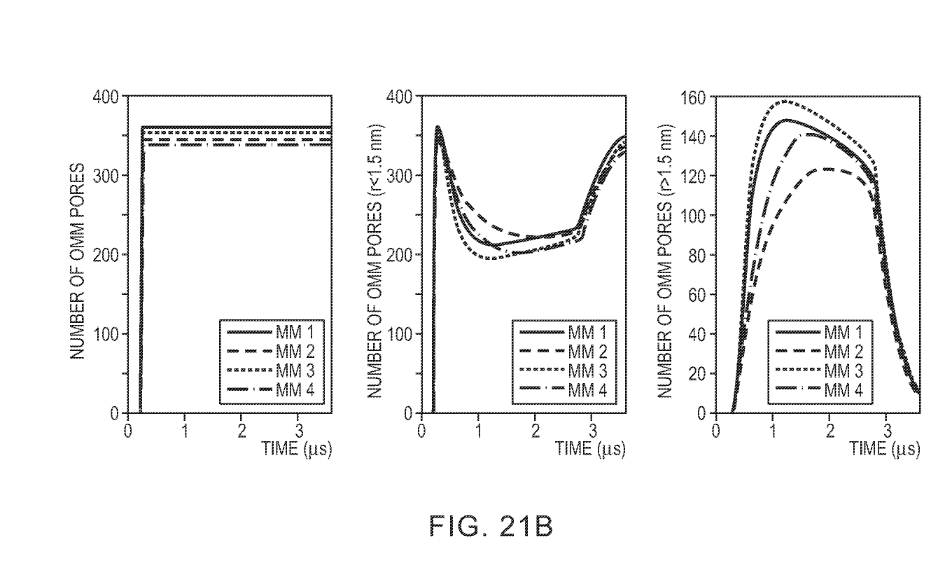

12 kV/cm, 3 μs trapezoidal pulse: This is an acceptable trapezoidal waveform for malting all cells apoptotic (see FIG. 21). FIG. 21 shows a 12 kV/cm, 3 μs trapezoidal pulse. The two circular cells are 10 μm and 20 μm in radius. In 2D, the four mitochondria are 2 μm×1 μm in size. The 121×121 geometry represents 60 μm×60 μm×0.5 μm simulation volume. EP models: asymptotic model for PM and IMM; SE model for OMM. Using the criterion of having at least 33 MOM pores with radii ≧1.5 nm that exist for at least 3 μs is equivalent to having at least 10 pores with radii ≧1.5 nm that exist for at least 10 μs. This assumes that the transient aqueous pores in the MOM limit the transport of released cyt-c. The approximate temperature rise for this pulse is 4° C. While the curves for the four MM “Number of OMM pores (≧1.5 nm)” is different for MM1-MM4, all have more than about 60 pores≧1.5 nm, which is enough to drive all cells apoptotic.

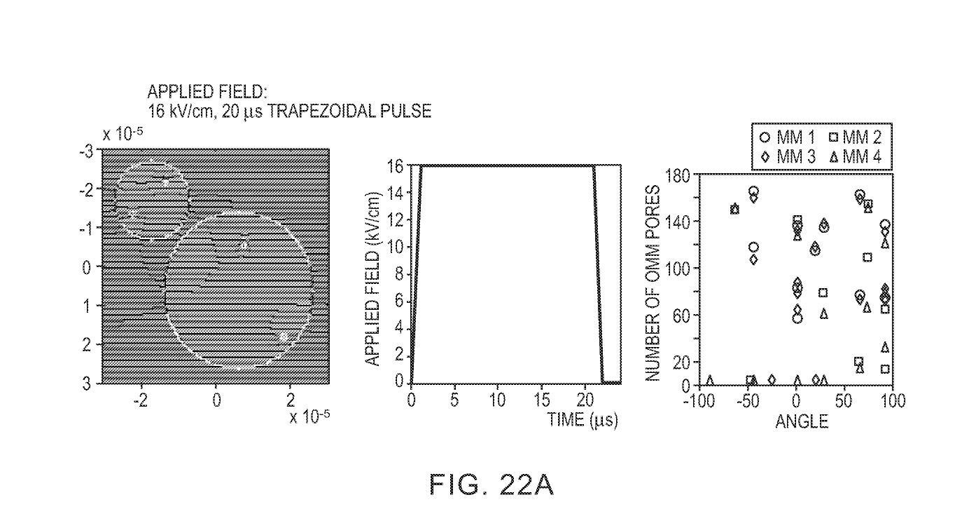

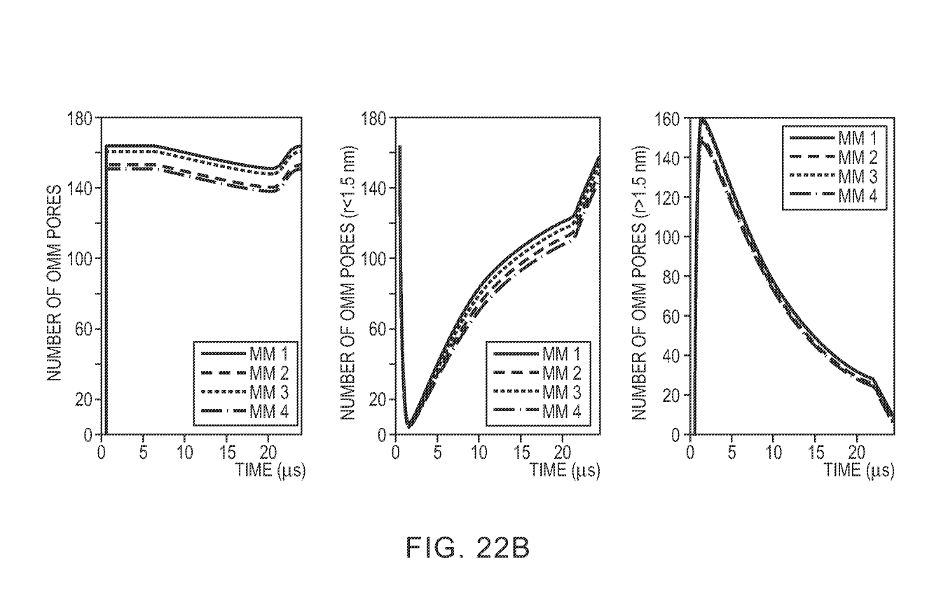

16 kV/cm, 20 μs trapezoidal pulse: This is an acceptable trapezoidal waveform for malting all cells apoptotic (see FIG. 22). FIG. 22 shows a 16 kV/cm, 20 μs trapezoidal pulse. The two circular cells are 10 μm and 20 μm in radius. In 2D, the four mitochondria are 2 μm×1 μm in size. The 121×121 geometry represents 60 μm×60 μm×0.5 μm simulation volume. EP models: asymptotic model for PM and IMM; SE model for OMM. This electric field waveform is expected to cause a temperature rise of about 7° C. For a normal core T (temperature) of 37° C., the maximum T will be about 44° C., which is 2° C. warmer than the “threshold” of 42° C. for accumulation of thermal damage by prolonged heating. If massive apoptosis is desired in a tumor that is initially close to 37° C., this level of heating may be acceptable. For peripheral tissue such as skin-related tissues, the normal T is often lower, e.g. 26° C. In this case a temperature rise of 7° C. to reach 33° C. should cause insignificant thermal damage. The use of multiple treatment sessions that are scheduled to allow phagocytic clearance of apoptotic cells is consistent with this amount of heating. Again, the curves for the four MM “Number of OMM pores (≧1.5 nm)” is somewhat different for MM1-MM4, but all have more than about 70 pores ≧1.5 nm for the first 10 μs of the pulse waveform. The maximum number of suitable pores for releasing cyt-c peaks in two of the MM at about 160 pores with ≧nm radii, and the other two MM peak at about 150. This electric field waveform thus predicts that all cells in the system will become apoptotic.

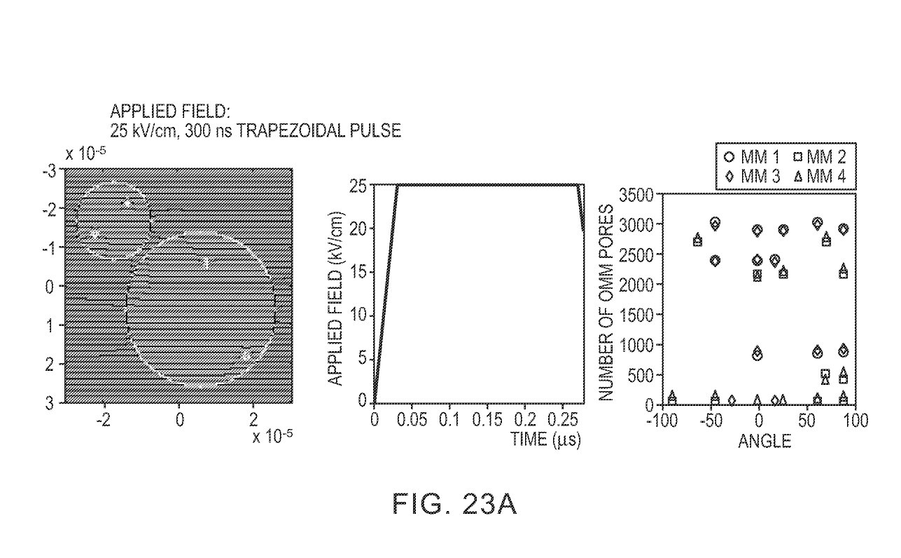

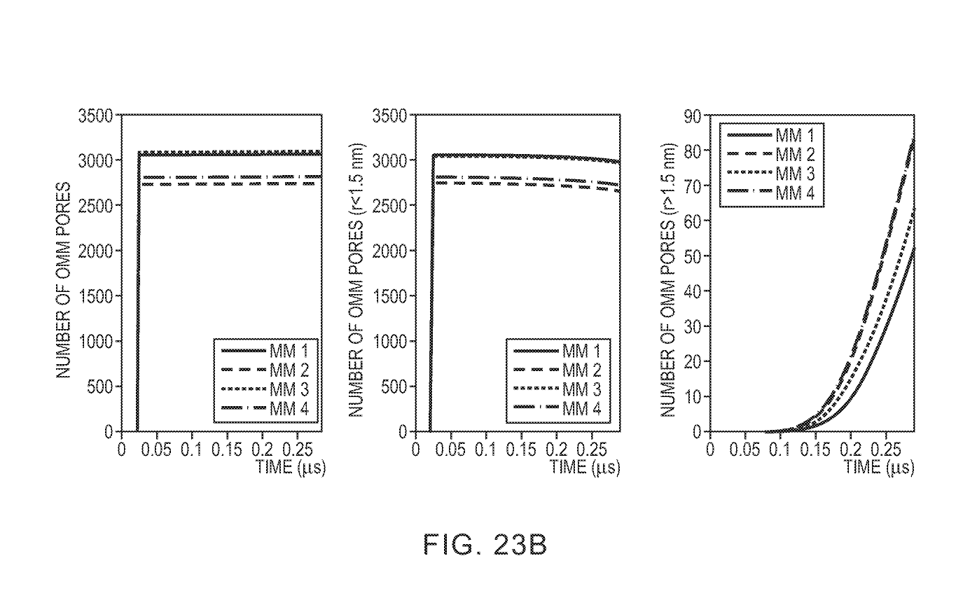

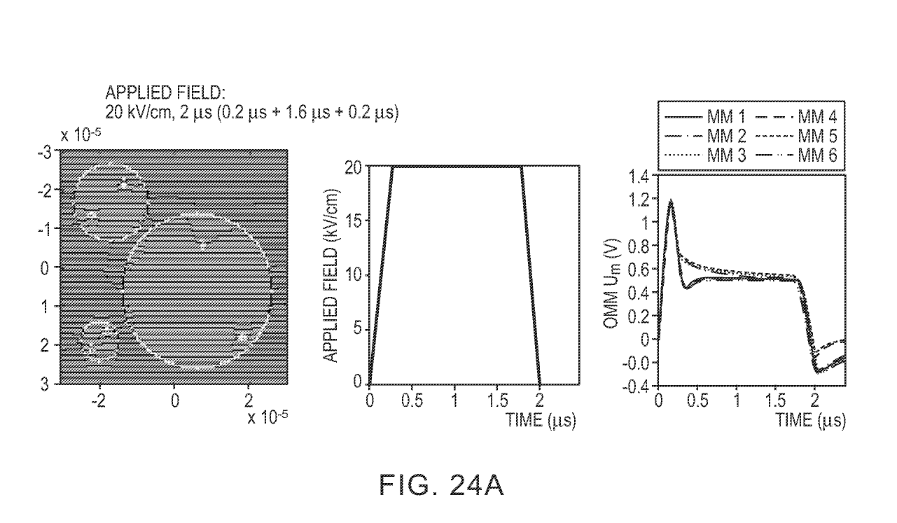

25 kV/cm, 300 μs trapezoidal pulse: This is waveform makes too few cells apoptotic (see FIG. 23). FIG. 23 shows a 25 kV/cm, 300 ns (nanosecond) trapezoidal pulse. The two circular cells are 10 μm and 20 μm in radius. In 2D, the four mitochondria are 2 μm×1 μm in size. The 121×121 geometry represents 60 μm×60 μm×0.5 μm simulation volume. EP models: asymptotic model for PM and IMM; SE model for OMM. This 300 ns electric field waveform approximates the pulse waveform used in early in vitro studies FIG. 9 shows that cyt-c was detected for a 3 PEF (pulsed electric field) at 1 s intervals, but the general mechanism suggested that calcium release from the endoplasmic reticulum (ER) is involved. There is no evidence of direct release of cyt-c from the intramembrane space by MOMEP. In the present result, the curves for the four MM “Number of OMM pores (≧1.5 nm)” are again somewhat different for the four MM (MOM). The curves climb from zero to a maximum of 90 at 0.275 μs (275 ns). Although this plot was inadvertently cut off before 300 ns, other results show that the cyt-c-releasing pores begin to shrink at the end of the pulse, so that the number of pores with ≧1.5 nm at 300 ns can be extrapolated to be about 100 for the maximum case and about 70 for the minimum case. But for 300 ns it is expected that approximately 300 pores with ≧1.5 nm would be needed. If the ≧2 nm radius for at least 10 μs criterion were used, then even more pores of this size would be needed. This in silico screening result therefore predicts that few cells would be made apoptotic by MOMEP causing cyt-c release, because the MOM pores are too small.

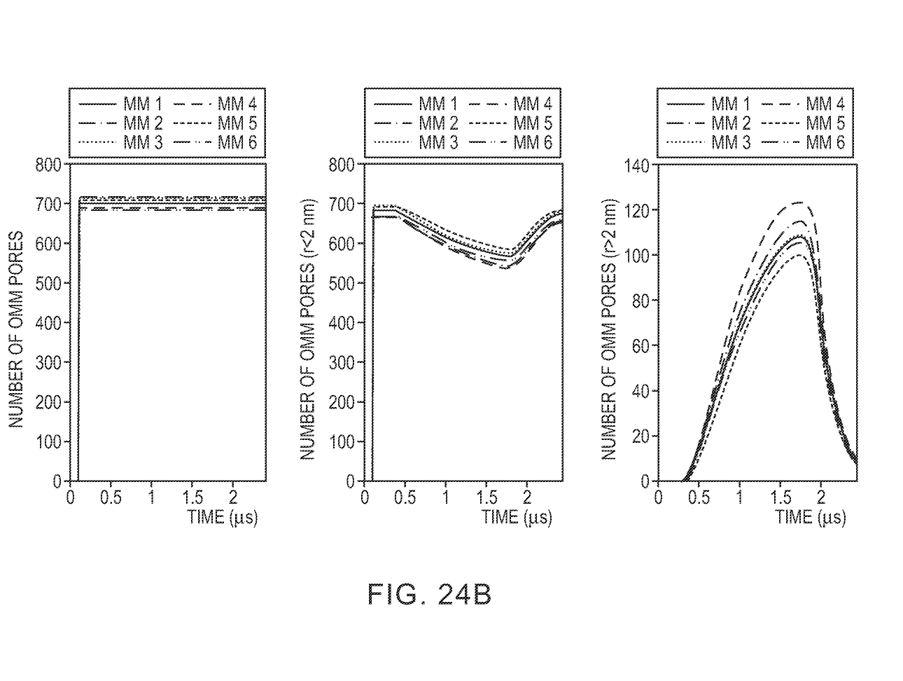

20 kV/cm, 2 μs, (0.2 μs+1.6 μs+0.2 μs): This is an acceptable trapezoidal waveform for making some cells apoptotic (see FIG. 24). FIG. 24 shows a 20 kV/cm, 2 μs, (0.2 μs+1.6 μs+0.2 μs) pulse. The three circular cells are 5, 10 and 20 μm in radius. The six mitochondria are 2 μm×1 μm in size. The 121×121 geometry represents 60 μm×60 μm×0.5 μm simulation volume. EP models: asymptotic model for PM and IMM; SE model for OMM. Trapezoidal pulses were applied. White regions indicate at least 10 pores in the lattice volume. This result is obtained from the three cell model, which has three thin (0.5 μm system model depth) cylindrical membranes, treated in 2D (two dimensions) as three descretized circles that give the location of the three plasma membranes (PMs). As described in Methods, each of the three cells also contains two mitochondrial models, whose construction is also disclosed in the Methods. Not only does this three cell model have cells with radii of 5, 10 and 20 μm, so that in this cell system model the cell size varies by a factor of 4, the displayed three cell model results also include the time dependent transmembrane voltage, Δψ(t) at one local area site on each MOM as “OMM Um (V)”. In addition to the notation Δψ(t), the transmembrane voltage (sometimes also termed “membrane potential”) is also often denoted by Uₘ(t) in the scientific and engineering literature.

As shown here the transmembrane voltage gives addition insight. Specifically Δψ(t) is seen to rise to a value of ˜1.2V in the first ˜0.2 μs for the electric field waveform used here. The relatively large trapezoidal electric field waveform rapidly charges the PM of each cell. At about 1.2 V for this rate of transmembrane voltage rise a non-linear burst of pore creations occurs. This burst dramatically increases the local PM electrical conductance. As a result the transmembrane voltage decreases over the time interval ˜0.2 to ˜0.4 μs. Two of the MM Δψ(t) curves drop monotonically to an approximate plateau value of about 0.5 V, the other four Δψ(t) curves drop farther and then resume charging the local membrane area that now has a much larger local conductance, and also reach the approximate plateau of about 0.5 V by approaching from below. This “spike” in Δψ(t) before joining the same approximate plateau is reversible electrical breakdown (REB), a dynamic voltage division that occurs because of electroporation. The term “breakdown” is misleading, in the sense that this event creates insignificant damage (not enough energy at about 1 V to ionize most molecules), and is better regarded as a local and transient high conductance state of the membrane. Usually the PM is the focus of attention, but here the main concern is the OMM (MOM). The Δψ(t) curves are consistent with results in previous publications hi which the asymptotic EP models were used in local membrane areas and also with the more recent use of a dynamic pore model. The panel with plots of the six MM curves (MM1-MM6) show curves for the predicted “Number of OMM pores (≧2 nm)”. This newer approximate criterion of needing pores with ≧2 nm is more stringent than the earlier approximate criterion of needing pores with radii ≧1.5 nm, and may also provide guidance to waveforms that will also release Smac/DIABLO and other death molecules that are larger than cyt-c.

These curves show that for the time interval of about 1 to 2 μs there are more than 100 such pores for some of the MM (MOM), and fewer for other of the MM. This electric field waveform will therefore make a subpopulation of cells apoptotic, rather than all cells apoptotic, which is relevant to electric field waveforms for multiply scheduled treatment sessions, in which the electric field is smallest in the initial session, and is made progressively larger in subsequent sessions. The temperature rise is about 4° C., a small value with insignificant thermal damage.

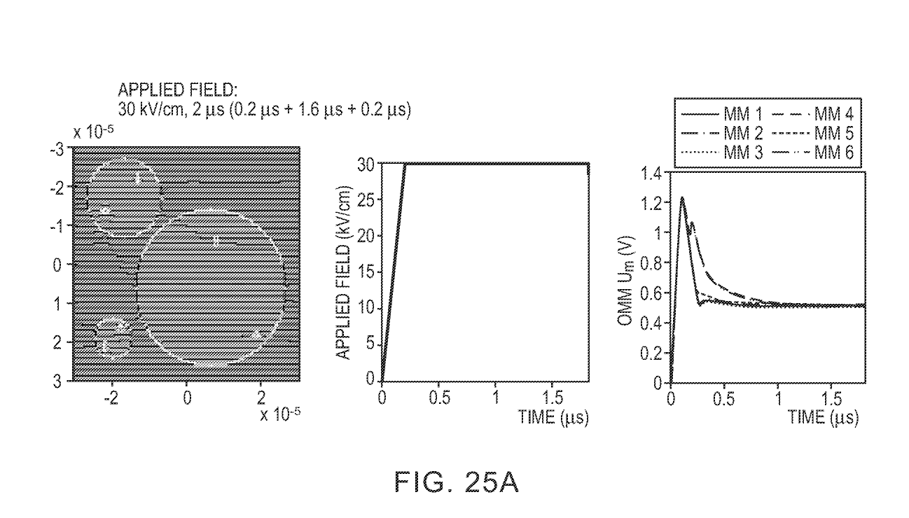

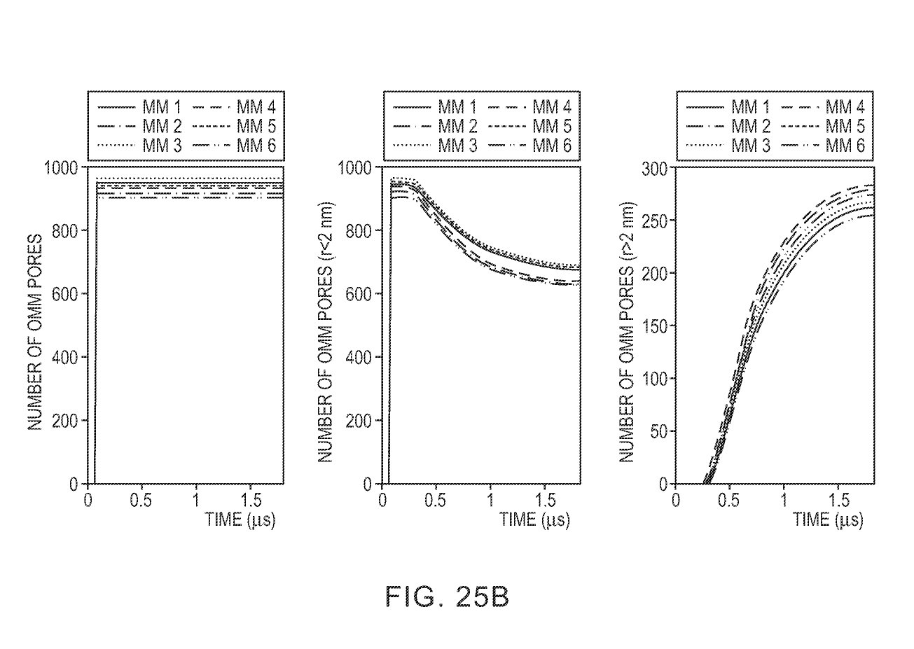

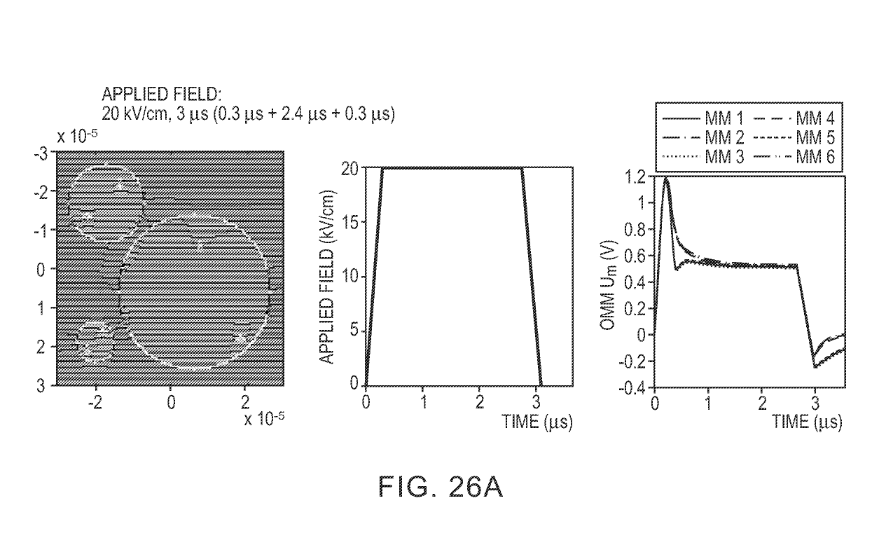

30 kV/cm, 2 μs, (0.2 μs+1.6 μs+0.2 μs): This is an acceptable trapezoidal waveform for malting all cells apoptotic (see FIG. 25). FIG. 25 shows a 30 kV/cm, 2 μs, (0.2 μs+1.6 μs+0.2 μs) pulse. The three circular cells are 5, 10 and 20 μm in radius. The six mitochondria are 2 μm×1 μm in size. The 121×121 geometry represents 60 μm×60 μm×0.5 μm simulation volume. EP models: asymptotic model for PM and IMM; SE model for OMM. Trapezoidal pulses were applied. White regions indicate at least 10 pores in the lattice volume.

This in silico screening test result is also obtained from the three cell model. In this case the Δψ(t) curves are more complex, a consequence of the large number of local models within the three cell system model that interact to yield a result. The initial OMM charging is nearly linear in time until a burst of pore creation occurs at ˜1.2V, but the post-spike behavior of Δψ(t) varies significantly for the individual local areas in the six OMM (MM1-MM6). Nevertheless all Δψ(t) tend towards the same approximate plateau value, again about 0.5 V.

The curves giving the “Number of OMM pores (≧2 nm)” are striking. These curves emerge from zero at about 3.5 μs, and rise steeply while then decreasing their rate of increase and spreading slightly apart, but all six MM (MOM; MM1-MM6) have more than 180 pores which can release cyt-c and somewhat larger death molecules.

The estimated temperature rise is about 9° C., which will cause insignificant thermal damage in tissue regions that have initial temperatures less than about 33° C. This electric field pulse waveform will male all cells apoptotic.

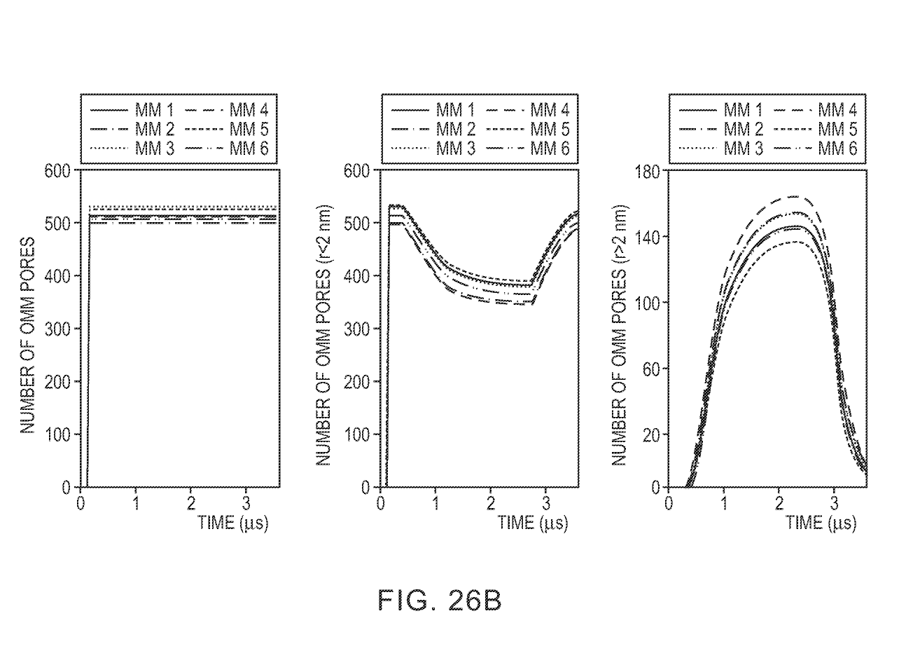

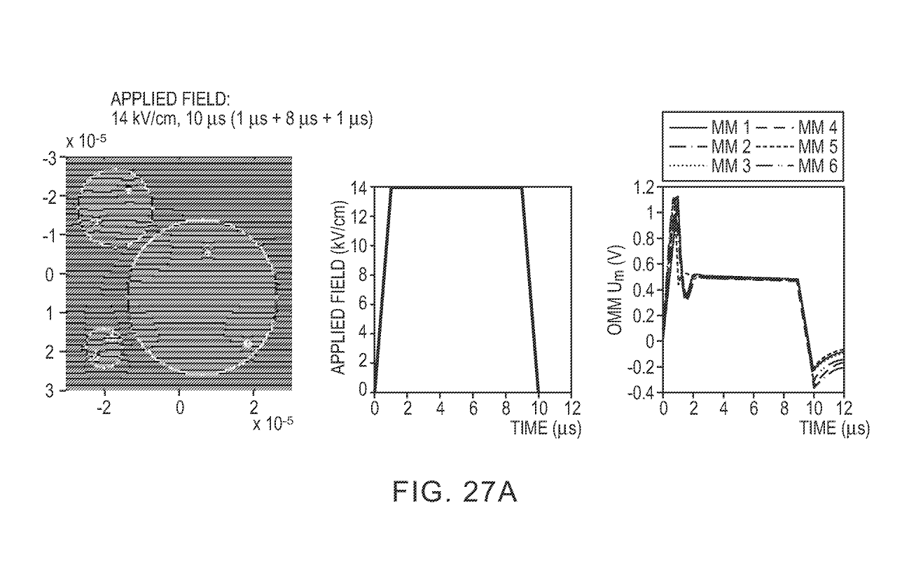

20 kV/cm, 3 μs, (0.3 μs+2.4 μs+0.3 μs): This is an acceptable trapezoidal waveform for malting all cells apoptotic (see FIG. 26). FIG. 26 shows a 20 kV/cm, 3 μs, (0.3 μs+2.4 μs+0.3 μs) pulse. The three circular cells are 5, 10 and 20 μm in radius. The six mitochondria are 2 μm×1 μm in size. The 121×121 geometry represents 60 μm×60 μm×0.5 μm simulation volume. EP models: asymptotic model for PM and IMM; SE model for OMM. Trapezoidal pulses were applied. White regions indicate at least 10 pores in the lattice volume. This three cell model result has a trapezoidal shaped electric field pulse waveform that has a slower rise time, with the applied field reaching its peak value of 20 kV/cm in 0.3 μs, maintaining the peak value for 2.4 μs, and then decreasing with a fall time of 0.3 μs. As in most cellular systems with significant electroporation, the various transmembrane voltages do not follow the applied field due to the highly non-linear behavior of membranes that shift voltage division within the cell systems' membranes as the local values of membrane conductance change non-linearly and hysteretically with time. For death molecule release the OMM (MOM) in each mitochondrion is of primary interest. Here the curves in the plot of the “Number of OMM pores (≧2 nm)” emerges from zero at about 0.4 μs, increases with some spreading of the individual MM curves (MM1 through MM6) such that at about 1 μs the number of cyt-c-releasing pores is about 100±20. This and larger numbers of pores with ≧2 nm exist until about 3 μs. The present approximate criterion is that 50 or more such pores are needed for this 2 μs interval. This condition is satisfied by the 100±20 pores. The estimated temperature rise is about 5.5° C. This electric field pulse waveform will make all cells apoptotic.

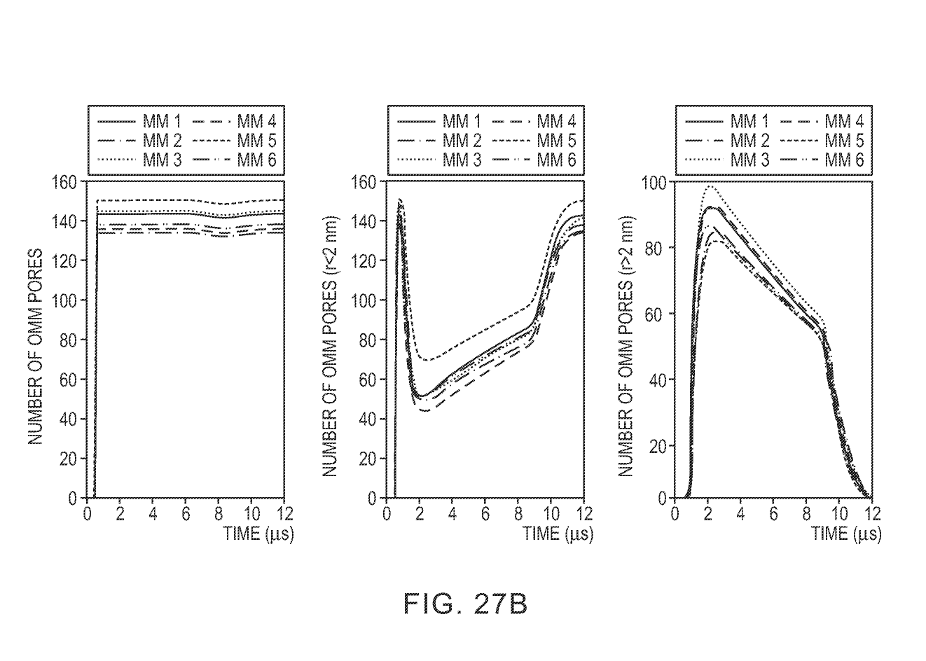

14 kV/cm, 10 μs, (1 μs+8 μs+1 μs): This is an acceptable trapezoidal waveform for malting all cells apoptotic (see FIG. 27). FIG. 27 shows a 14 kV/cm, 10 μs, (1 μs+8 μs+1 μs) pulse. The three circular cells are 5, 10 and 20 μm in radius. The six mitochondria are 2 μm×1 μm in size. The 121×121 geometry represents 60 μm×60 μm×0.5 μm simulation volume. EP models: asymptotic model for PM and IMM; SE model for OMM. Trapezoidal pulses were applied. White regions indicate at least 10 pores in the lattice volume. This three cell model system result also shows that electroporation occurs early in the electrical exposure for this electric field pulse waveform, a trapezoidal pulse with rise and fall times of 1 μs and a flat peak of duration 8 μs and magnitude 14 kV/cm. The plot of curves for the “Number of OMM pores (≧2 nm)” exhibits a rapid rise starting from zero at about 0.8 μs, with more than about 50 pores with radii ≧2 nm in the time interval 1 μs to 9 μs. The present criterion requires 10 or more such pores existing for 10 μs or longer. Again, based on pore transport of released cyt-c being the rate limiting step, the same level of release can be accomplished in the 8 μs interval when 12 or more cyt-c-releasing pores are present in each MOM (OMM) of the six mitochondlia of this multicellular model. Inspection of the “Number of OMM pores (≧2 nm)” shows that approximately 40 or more such pores are present. The six MOM (OMM) values peak in the range 80 to 100, all well above 40. The temperature rise is approximately 6° C. This applied electric field waveform will make all cell apoptotic.

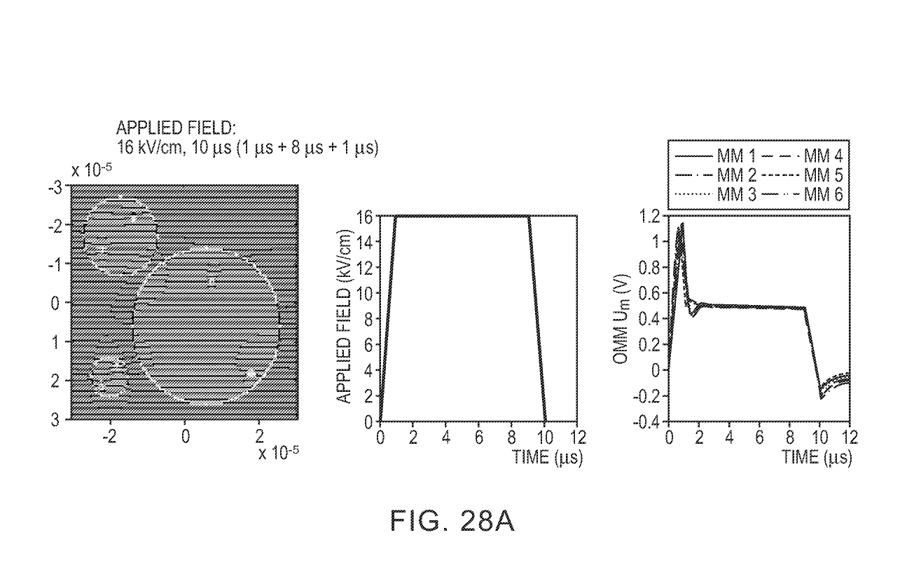

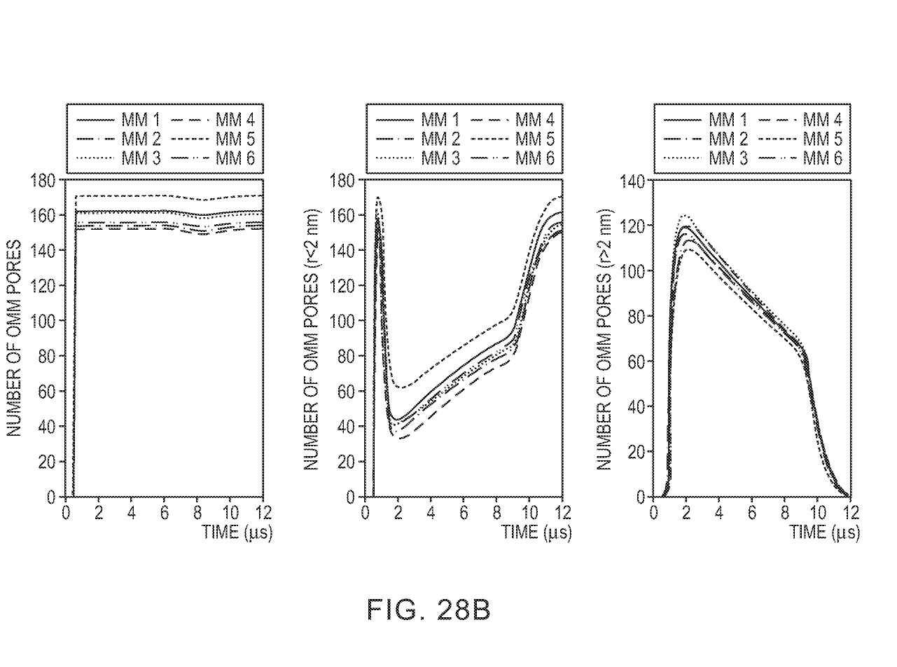

16 kV/cm, 10 μs, (1 μs+8 μs+1 μs): This is an acceptable trapezoidal waveform for malting all cells apoptotic (see FIG. 28). FIG. 28 shows a 16 kV/cm, 10 μs, (1 μs+8 μs+1 Aμs) pulse. The three circular cells are 5, 10 and 20 μm in radius. The six mitochondlia are 2 μm×1 μm in size. The 121×121 geometry represents 60 μm×60 μm×0.5 μm simulation volume. EP models: asymptotic model for PM and IMM; SE model for OMM. Trapezoidal pulses were applied. White regions indicate at least 10 pores in the lattice volume. This result uses all applied electric field waveform for which the waveform magnitude is slightly larger than the 14 kV/cm, above. The temperature rise is therefore a factor (16/14)2)=1.31 larger, viz. ˜8° C. This magnitude temperature rise may actually cause no difficulties in treatments of non-peripheral (deeply located, away from the body's surface) unwanted tissues that have an initial T of 37° C. In this case of peripheral tissues such as skin-associated tissues, or even adipose tissues, a temperature rise of 8° C. will be acceptable. The plots of “Number of OMM pores (≧2 nm)” shows that approximately 70 or more such pores are present during the 8 μs between ˜1 μs and ˜9 μs. To make cells apoptotic our criterion requires at least 12 cyt-c releasing pores be present during this 8 μs. Inspection of the plots shows that this condition is readily met. Although the six curves for MM1-MM6 are clearly different in detail, they all have more than about 70 such pores. The maximum peak value is above 120 such pores; the minimum peak has about 110 such pores. This applied electric field waveform will male all cells apoptotic.

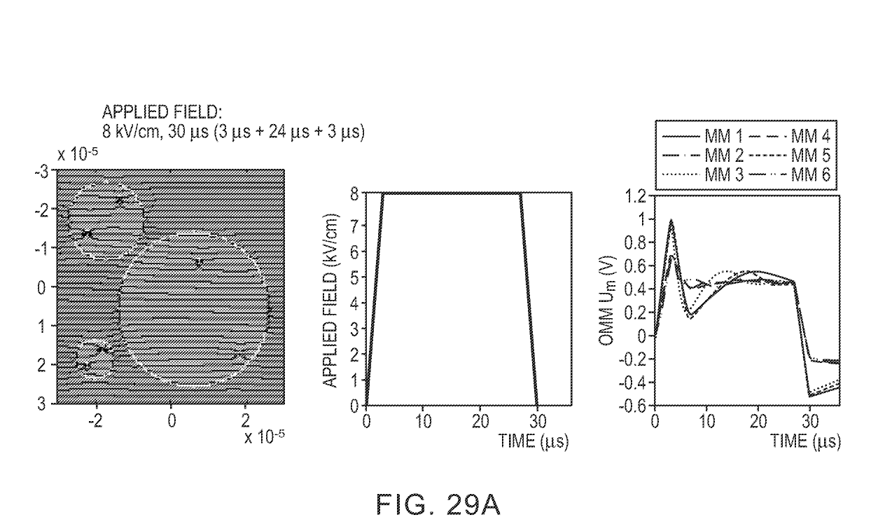

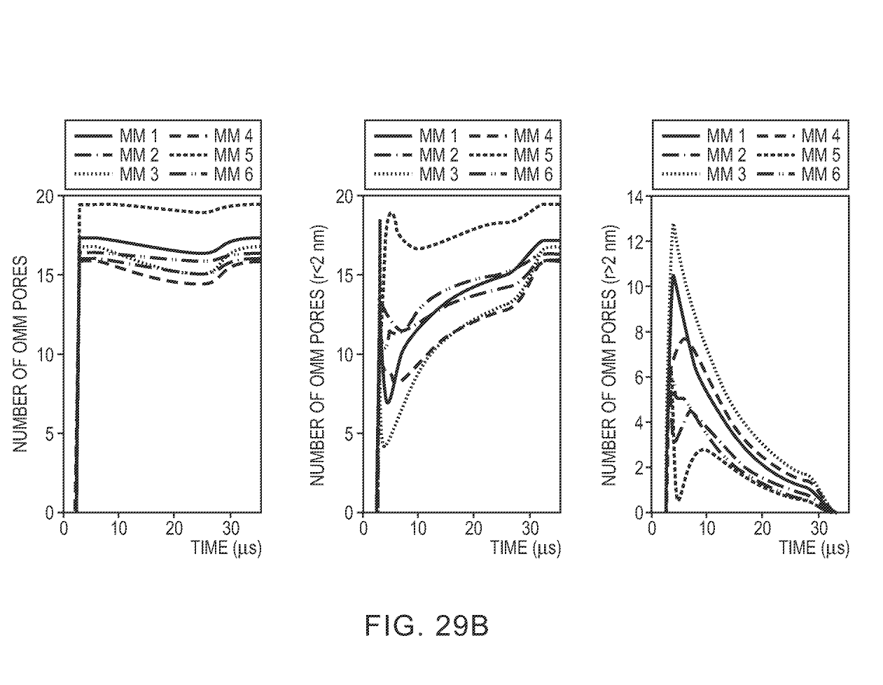

kV/cm, 30 μs, (3 μs+24 μs+3 μs): This is a barely acceptable trapezoidal waveform for making a few cells apoptotic (see FIG. 29). FIG. 29 shows a 8 kV/cm, 30 μs, (3 μs+24 μs+3 μs) pulse. The three circular cells are 5, 10 and 20 μm in radius. The six mitochondria are 2 μm×1 μm in size. The 121×121 geometry represents 60 μm×60 μm×0.5 μm simulation volume. EP models: asymptotic model for PM and IMM; SE model for OMM. Trapezoidal pulses were applied. White regions indicate at least 10 pores in the lattice volume. This three cell model result shows how complicated the electrical and poration response can be in a multicellular system. The Δψ(t) plots show that for the OMM (MOM) of the model, EP is barely achieved in some cases (small spikes that indicate REB), with only one spike reaching 1V. The curves that show the “Number of OMM pores (≧2 nm)” indicates more directly than only MM3 (OMM or MOM) is close to having enough pores with ≧2 nm for 10 μs. This dotted curve rises to ˜13 such pores, but then falls slowly out to 30 μs, so that the net (time integrated) release of cyt-c may reach the value associated with our approximate criterion of 100 pore its (corresponds to 10 cyt-c-releasing pores for 10 μs). The estimated temperature rise is ˜12° C., which is acceptable for peripheral cooler tissues where the treatment may involve both medical and cosmetic concerns, and may also be acceptable for malignant tissue removal if this unwanted tissue is located away from the body surface. This applied electric field waveform will male some of the cells apoptotic, and is an example of a waveform that may be suitable for beginning a multiple session treatment program, where time is provided for phagocyte migration and subsequent clearance of apoptotic cells before secondary necrosis can occur.

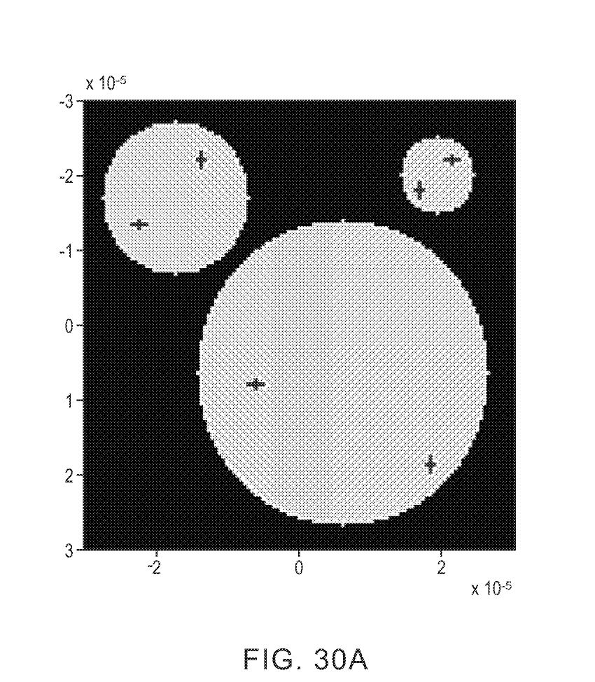

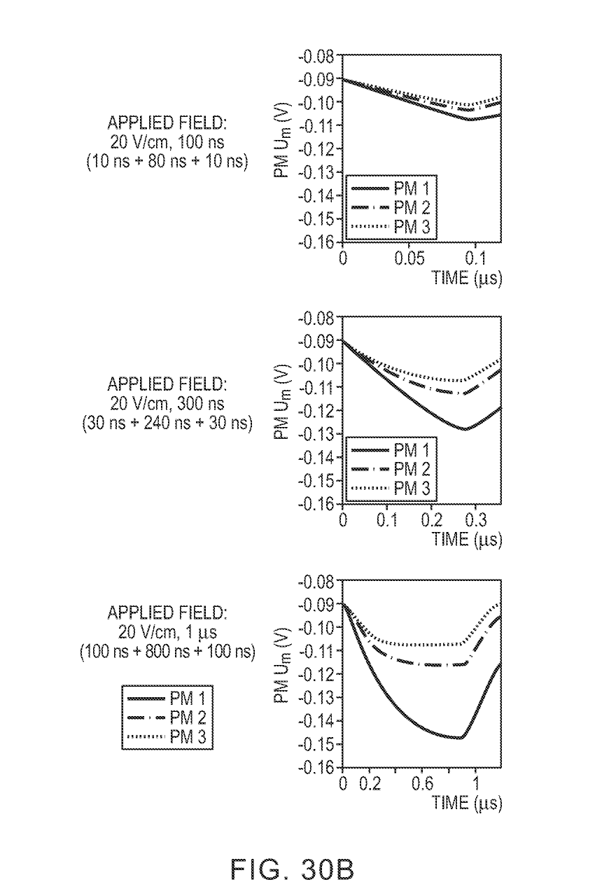

20V/cm small magnitude trapezoidal pulses: Passive charging behavior for partial model validation. FIGS. 30A-C show 20 V/cm, small magnitude trapezoidal pulses with passive charging behavior for partial model validation. As shown in FIG. 30A, the three circular cells are 5, 10 and 20 μm in radius. The six mitochondria are 2 μm×1 μm in size. The 121×121 geometry represents 60 μm×60 μm×0.5 μm simulation volume. EP models: asymptotic model for PM and IMM; SE model for OMM. Trapezoidal pulses were applied.

First the three cell model geometry is shown (see FIG. 30A). As shown in FIG. 30A, the vertical and horizontal axes are both ±30 μm. The model shows the three descretized circles that locate the plasma membranes (PMs). Within each cell there are two mitochondria with size 2 μm (length)×1 μm (width)×0.5 μm (depth). The dynamic local EP model is assigned to all local membrane areas in the PM and the OMM (MOM) and IMM (MIM), but for the very small magnitude applied electric field waveform used here to test the passive response of the model, negligible pores are created, and the main result is passive charging of the membranes. Here, as a further validation of our modeling methods the top-most (“anodic pole”) local area of each of the three cell PMs is considered, with Δψ(t) at these local areas plotted as “PM Um (V)” for trapezoidal pulse waveform durations of 100 ns, 300 ns, 1 μs, 3 μs and 10 μs, with the rise, peak duration and fall times indicated above each figure (see FIGS. 30B-C).

These three cell model results are consistent with the traditional analysis of passive cylindrical membranes, whose behavior is closely related to passive spherical cell membranes. Note that a purely passive model is not used. Instead the complete model that exhibits varying and often complex EP behavior distributed within the three cell model is used. The only difference is that the applied electric field waveform (20V/cm) is 2 to 3 orders of magnitude smaller. Thus, the model itself properly yields solutions that are in general agreement with the traditional passive models if small magnitude waveforms are used. The 20V/cm, 3 μs figure is particularly useful, as it shows the smaller (radius r_{cell}=5 μm) cell charging most rapidly, followed by the (r_{cell}=10 μm) cell, with the largest cell (radius r_{cell}=20 μm) raising the transmembrane voltage to reach the fully charged value (transmembrane voltage flat peal) most slowly. Slight differences between the three cell model with closely spaced cells and the traditional isolated (cell surrounded by infinite extracellular medium) are expected, but the main features of the small magnitude electric field waveform response are in reasonable agreement with the traditional, isolated cell models.

### Example 4

### Pulser

In a further aspect, the invention provides a pulse generator (pulser) that generates high-voltage, high-current pulses with controlled rise and fall times. In one embodiment, the pulser includes various circuit elements and modules within a housing. In part, in one embodiment, the pulsar includes a plurality of electrically isolated controlled switches each containing a capacitor for supplying the pulse energy. Each module can develop up to 4 kV pulses at currents in excess of 100 amps. Nominal pulse durations range from 10 to 1000 microseconds with rise and fall times ranging between 1 and 10 microseconds. Each module is electrically isolated using high-voltage vacuum relays and optical isolators.

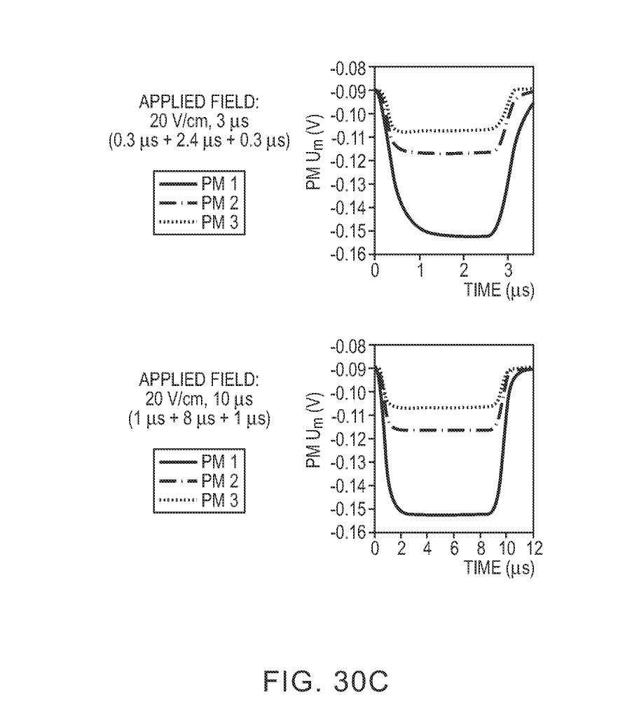

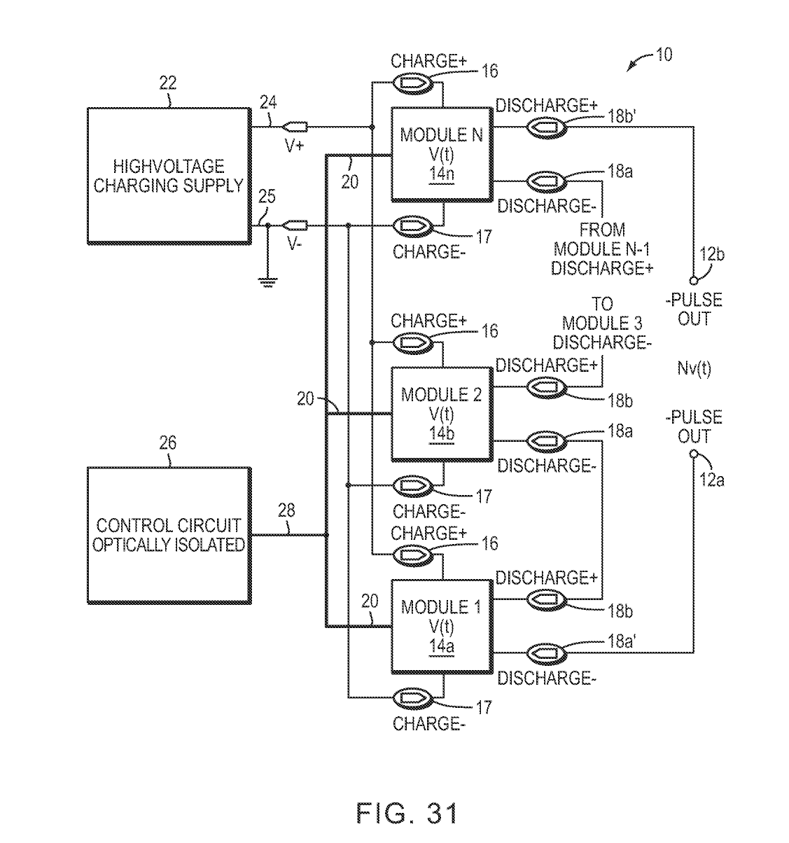

FIG. 31 depicts a block diagram of a variable high voltage high current pulser 10, in accordance with an illustrative embodiment of the present invention. Referring to FIG. 31, a plurality of modules 14a-14n (Module 1 . . . Module N) are placed in series. Any number of modules (N) can be used, such as, for example, N=1, 2, 3, 4, 5, 6, 7, 8, 9, 10, 15, 20 or more modules. The pulser includes a first discharging terminal 12a and a second discharging terminal 12b, a plurality of switched current supplying modules 14a-14n, a high voltage charging supply 22 which has a high voltage supply charging terminal 24, and a control circuit 26 which has a control bus 28.

Each switched current supplying module 14a-14n can include a positive charging terminal 16, a negative charging terminal 17, a first discharging terminal 18a, a second discharging terminal 18b, and a control bus 20. The positive charging terminal 16 of each of the plurality of switched current supplying modules 14a-14n is connected to the high voltage supply positive charging terminal 24 of the high voltage power supply 22. The negative charging terminal 17 of each of the plurality of switched current supplying modules 14a-14n is connected to the high voltage supply negative charging terminal 25 of the high voltage power supply 22. The control bus 20 of each of the plurality of switched current supplying modules 14a-14n is connected to the control bus 28 of the control circuit 26. The second discharging terminal 18b of each of the plurality of switched current supplying modules 14a-14n, except a last of the plurality of switched current supplying modules 14a-14n, is connected to the first discharging terminal 18a of the subsequent one of the plurality of switched current supplying molecules 14a-14n. The second discharging terminal 18b′ of the last of the plurality of switched current supplying molecules 14a-14n connects to (or is) the second discharging terminal 12b of the pulser 10, and the first discharging terminal 18a′ of the plurality of switched current supplying molecules 14a-14n connect to (or is) the first discharging terminal 12a of the pulser 10. In some embodiments, the first discharging terminal 12a and second discharging terminal 12b are in electrical communication with first and second output electrodes, respectively, which electrodes are configured for contact with and delivering pulses to a patient tissue. In one embodiment, the relevant discharging terminals or terminal are disposed within a treatment head or other treatment region contacting device. Breakdown limitations of the relays and optical isolators provide a practical limit to the voltage developed by the purser. In one embodiment, all modules are controlled by a common control circuit 26 providing identical but isolated signals to each module.

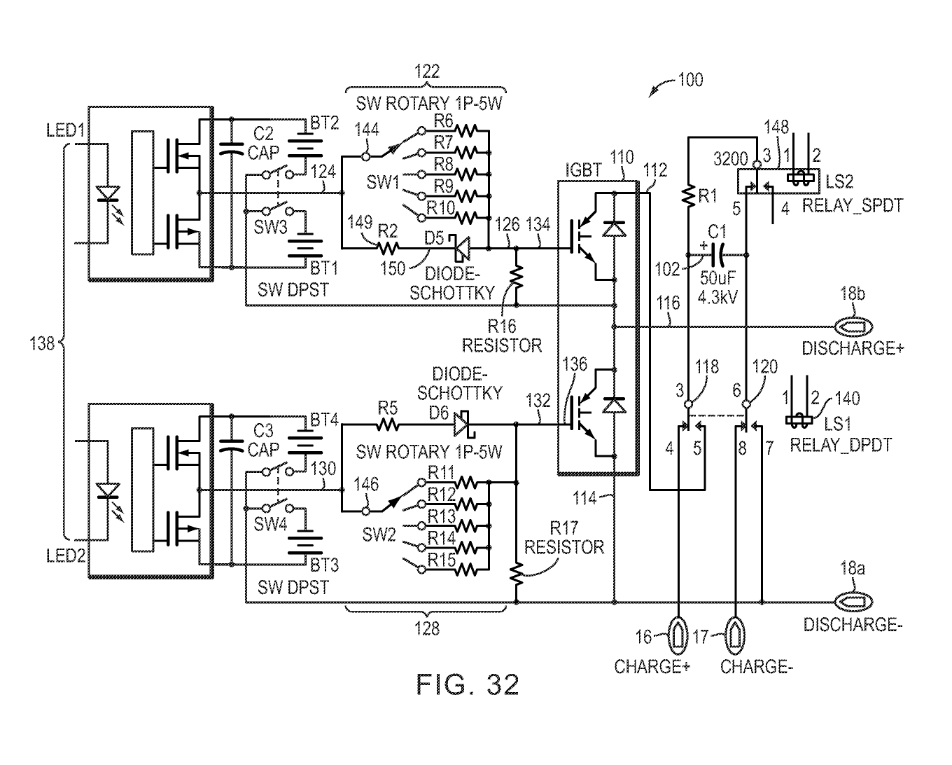

FIG. 32 depicts a schematic diagram of a switched current supplying module 100, in accordance with an illustrative embodiment of the present invention. Referring to FIG. 32, in some embodiments, each of the plurality of switched current supplying molecules 14a-14n can further include a capacitor 102, a half bridge 110, a first switch 118, and a second switch 120. Energy is stored in the storage capacitor 102 (C1), which can be, for example, a 50 microfarad, 4300 volt capacitor. Half bridge 110 includes a first terminal 112, a second terminal 114, and a third terminal 116. First switch 118, in a first state, connects one side of capacitor 102 to the charging terminal of the switched current supply module 100. In a second state, the first switch connects 118 the same side of capacitor 102 to the first terminal 112 of the half bridge 110. Second switch 120, in a second state, connects the other side of capacitor 102 to the second terminal 114 of the half bridge 110. Third terminal 116 of the half bridge 110 is the second discharge terminal 18b of the switched current supplying module 100.

With further reference to FIG. 32, in some embodiments, each of the each of the plurality of switched current supplying molecules 14a-14n can further include a rise-time control module 122 having an input terminal 124 and an output terminal 126, and a fall-time control module 128 having an input terminal 130 and an output terminal 132. A fourth terminal 134 of half bridge 110 connects to the output terminal 126 of the rise-time control module 122. A fifth terminal 136 of half bridge 110 connects to the output terminal 132 of the fall-time control module 128. Input terminal 124 of the rise-time control module 122 is in electrical communication with the control bus 138 of the switched current supplying module 100.

Charging terminals 16 (CHARGE+) and 17 (CHARGE−) connect the capacitor 102 to a current-limited high voltage charging circuit through the normally closed contacts of a high-voltage vacuum relay 140 (LS1). High-voltage normally-closed relay 148 connects a high voltage (e.g., 3200 ohm) resistor across capacitor 102 when the circuit is inactive. Using an insulated-gate bipolar transistor (IGBT) within the half-bridge 110 provides a controlled, low-impedance pulse during active pulse generation. Rise-time switch 144 (SW1) controls the pulse rise-time of the output pulse by limiting the gate-current of the upper IGBT in the half-bridge 110. Fall-time switch 146 (SW2) controls the pulse fall-time of the output pulse by limiting the gate current of the lower IGBT in the half-bridge 110. Gate control current is provided by batteries turned on and off with rotary switches. All rotary switches are electrically isolated by means of insulated shafts and are meant to be operated only while the pulser is inactive.

With further reference to FIG. 32, a toggle switch activates a relay 148 (LS2), removing the safety discharge resistor from the module's storage capacitor 102 (C1). When a relay 148 (LS2) is activated, a push-button is enabled which initiates a pulse sequence. The sequence turns on relay 140 (LS1) which disconnects the capacitor from the high-voltage charging supply 22 and connects it to the IGBT half-bridge 110. After a delay allowing relay 140 (LS1) to operate, the upper IGBT is turned on. Turn on time is determined by the resistance selected by rise-time switch 144 (SW1) generating a positive pulse. The pulse is turned off by first rapidly turning off the upper IGBT through resistor (R2) 149 and diode (D5) 150. A pulse delay allows the upper IGBT to turn substantially off before operating the lower IGBT.

After the upper-turn-off delay, the lower IGBT is turned on through a resistor selected by fall-time switch 146 (SW2). The limited gate current provides controlled fall-time. After the pulse is completed the lower IGBT is turned off and relay 140 (LS1) is deactivated and the storage capacitor 102 (C1) is allowed to recharge.

### Variables

Unless otherwise stated, variables and parameters are defined as described below:

| Parameter | Description |
|---|---|
| Nx × Ny | Number of nodes |
| Lx × Ly × Lz | System model volume |
| r_{cell} | Cell radius |
| l | Lattice spacing |
| dₘ | Membrane thickness |
| εₑ | Electrolyte relative permittivity |
| εₘ | Membrane relative permittivity |
| εₗ | Local lipid relative permittivity |
| σe | Electrolyte conductivity |
| T | Temperature |
| q | Electroporation constant |
| α | Pore creation rate constant |
| Nₒ | Baseline pore density |
| Vₑₚ | Electroporation voltage scale |
| wₒ | Born energy barrier |
| H | Pore hindrance factor |
| η | Relative entrance of pore (form factor) |
| rₚ | Effective pore radius |
| d_{ER} | Diameter of ER |
| d_{IMM} | Mitochondria intermembrane space thickness |
| d_{NM} | Nucleus intermembrane space thickness |
| Vᵢₚ | Resting potential |
| Gᵢₚ | Local ion pump conductance |
| σₘ | Membrane conductivity |
| f_{p, A} | Protein fraction by area |
| f_{A} | Membrane area correction factor |
| NM | Number of local membrane models. |

It should be appreciated that various aspects of the claimed invention are directed to subsets and substeps of the techniques disclosed herein. Further, the terms and expressions employed herein are used as terms of description and not of limitation, and there is no intention, in the use of such terms and expressions, of excluding any equivalents of the features shown and described or portions thereof, but it is recognized that various modifications are possible within the scope of the invention claimed. Accordingly, what is desired to be secured by Letters Patent is the invention as defined and differentiated in the following claims, including all equivalents.

What is claimed is:

1. A method for selecting parameters of an electrical pulse for electroporation to induce apoptosis in a tissue in need of therapeutic removal in a patient, comprising the steps of:
generating an equivalent electrical model of the tissue;
calculating from said model the number of death molecules, which are released from all MOM pores in each cell in said tissue for an electrical pulse; and
selecting an electric pulse that will produce a number of released death molecules sufficient to initiate apoptosis in said tissue,
wherein the calculations are performed using a computer.

2. The method of claim 1, wherein the death molecules comprise at least one of Cytochrome-c, Smac/DIABLO, AIF, EndoG, and Omi/HTRAN2.

3. The method of claim 1, wherein the electric pulse has a time constant, field strength, rise time, fall time, and pulse form.

4. The method of claim 1, wherein the number of death molecules released is sufficient to achieve caspase-3 activation in the tissue.

5. The method of claim 1, wherein the calculation step is performed using a transport lattice equivalent electrical model of the tissue.

6. The method of claim 1, wherein the step of calculating the number of released death molecules is performed using death molecule current through a MOM pore.

7. The method of claim 1, wherein said death molecule current for cytochrome-c is given by
$${I}_{pore}^{cyt - c} = {\frac{π {r}_{p}^{2} {F}^{2} {Δψ}_{m}^{MOM} {D}_{cyt - c} {z}_{cyt - c}^{2}}{{RTd}_{m}} [ cyt - c ]}_{i} ,$$

8. (canceled)

9. A method for treating a disease by inducing apoptosis in a tissue in need of therapeutic removal in a patient, the method comprising:
generating an equivalent electrical model of the tissue;
calculating from said model the number of death molecules, which are released from all MOM pores in each cell in said tissue for an electrical pulse;
selecting an electric pulse having a time constant, field strength, and pulse form that will produce a number of released death molecules sufficient to initiate apoptosis in said tissue; and
applying said electric pulse to said tissue.

10. The method of claim 9 wherein the pulse form of the electric pulse is selected from the group consisting of exponential, trapezoidal, and bipolar.

11. The method of claim 9, wherein the duration of the electric pulse is time constant τₚᵤₗₛₑ greater than 1.0 microsecond and less than 100 microsecond.

12. The method of claim 9, wherein the magnitude of the applied electric field E⁰ₐₚₚ is greater than 5 kV/cm and not more than about 40 kV/cm.

13. The method of claim 9, wherein the death molecule released is one or more selected from the group consisting of cytochrome-c, Smac/DIABLO, AIF, EndoG, Omi/HTRA2, and combinations thereof.

14. (canceled)

15. (canceled)

16. The method of claim 9, wherein a plurality of electric pulses are administered at a frequency of at least about one hour to at least about several days.

17. (canceled)

18. The method of claim 9, wherein the electric pulses are administered in a number and at a frequency sufficient to allow time to allow phagocytes to engulf apoptotic cells.

19. (canceled)

20. A computer-usable medium having computer readable instructions stored thereon to perform a method for selecting parameters of an electrical pulse for electroporation sufficient to induce apoptosis in a tissue in need of therapeutic removal in a patient, the method comprising:
generating an equivalent electrical model of the tissue in need of therapeutic removal;
calculating from said model the number of death molecules, which are released from all MOM pores in each cell in said tissue for an electrical pulse; and
selecting an electric pulse that will produce a number of released death molecules sufficient to initiate apoptosis in said tissue.

21. An apparatus for treating a patient by causing apoptosis in a tissue in need of therapeutic removal, said apparatus comprising:
means for generating an equivalent electrical model of the tissue;
means for calculating from said model the number of death molecules, which are released from all MOM pores in each cell in said tissue for an electrical pulse; and
means for selecting an electric pulse that will produce a number of released death molecules sufficient to initiate apoptosis in said tissue.

22-51. (canceled)

52. A variable high voltage high current pulse generator comprising:
a first discharging terminal and a second discharging terminal;
a plurality of switched current supplying modules, each of the switched current supplying modules comprising:
a positive charging terminal and a negative charging terminal;
a first discharging terminal and a second discharging terminal; and
a control bus;
a high voltage charging supply having a high voltage supply positive charging terminal and a negative charging terminal; and
a control circuit having a control bus,
wherein the positive charging terminal of each of the plurality of switched current supplying modules is connected to the high voltage supply positive charging terminal of the high voltage power supply,
wherein the negative charging terminal of each of the plurality of switched current supplying modules is connected to the high voltage supply negative charging terminal of the high voltage power supply,
wherein the several control means of the control bus of each of the plurality of switched current supplying modules is connected to the control bus of the control circuit,
wherein the second discharging terminal of each of the plurality of switched current supplying modules except a last of the plurality of switched current supplying modules is connected to the first discharging terminal of the subsequent one of the plurality of switched current supplying modules,
wherein the second discharging terminal of the last of the plurality of switched current supplying modules is the second discharging terminal of the high voltage high current pulser, and
wherein the first discharging terminal of the first of the plurality of switched current supplying modules is the first discharging terminal of the high voltage high current pulser.

53. The variable high voltage high current pulse generator of claim 52, wherein each of the plurality of switched current supplying modules further comprises:
a current source;
a half bridge comprising:
a first terminal,
a second terminal, and
a third terminal;
a first switch, in a first state, connecting one side of the current source to the charging terminal of the switched current supplying module; and, in a second state, connecting the same side of the current source to the first terminal of the half bridge;
a second switch, in the second state, connecting the other side of the current source to the second terminal of the half bridge; and
the third terminal of the half bridge being the second discharge terminal of the switched current supplying module.

54. The variable high voltage high current pulse generator of claim 53, wherein each of the plurality of switched current supplying modules further comprises:
a rise-time control module having an input terminal and an output terminal; and
a fall-time control module having an input terminal and an output terminal,
wherein the half bridge further comprises a fourth terminal and a fifth terminal,
wherein the fourth terminal of the half bridge is connected to the output terminal of the rise-time control module; and
wherein the fifth terminal of the half bridge is collected to the output terminal of the fall-time control module.

55. The variable high voltage high current pulse generator of claim 53,
wherein the input terminal of the rise-time control module is in electrical communication with the control bus of the switched current supplying modules, and
wherein the input terminal of the fall-time control module is in electrical communication with the control bus of the switched current supplying modules.

56-59. (canceled)
